SEQUENCE LISTING (87 sequences)

SEQ ID NO: 1 - DNA, 131 bp, Homo sapiens
  source 1..131: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 2 - DNA, 393 bp, Homo sapiens
  source 1..393: mol_type = other DNA
ctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatgg

SEQ ID NO: 3 - DNA, 53 bp, synthetic construct
  source 1..53: mol_type = other DNA
gggctgggcataaaagtcagggcagagccatctattgcttacatttgcttctg

SEQ ID NO: 4 - DNA, 68 bp, synthetic construct
  source 1..68: mol_type = other DNA
gaggtaggcgtgtacggtgggaggcctatataagcagagctcgtttagtgaaccgtcagatcgcctgg

SEQ ID NO: 5 - DNA, 68 bp, synthetic construct
  source 1..68: mol_type = other DNA
gaggtaggcgtgtacggtgggaggcctatataagcagagctggtttagtgaaccgtcagatcgcctgg

SEQ ID NO: 6 - DNA, 120 bp, synthetic construct
  source 1..120: mol_type = other DNA
gattcagccgggagcttagggaggggaggtcacttcataagggcctggggggggagttggagccacgagtcgtccagccggagccccgtgtggctgagctccggcctcagaagcatcccc

SEQ ID NO: 7 - DNA, 120 bp, synthetic construct
  source 1..120: mol_type = other DNA
gattcagccgggagcttagggaggggaggtcacttcataagggcttggggggggagttggagccacgagtcgtccagccggagccccgtgtggctgtgctccggcctcagaagcatcccc

SEQ ID NO: 8 - DNA, 867 bp, synthetic construct
  source 1..867: mol_type = other DNA
caggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 9 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 10 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 11 - DNA, 1299 bp, synthetic construct
  source 1..1299: mol_type = other DNA
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 12 - DNA, 1056 bp, synthetic construct
  source 1..1056: mol_type = other DNA
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaa

SEQ ID NO: 13 - DNA, 50 bp, synthetic construct
  source 1..50: mol_type = other DNA
gaagctacccctaacacactattctacacacagaaaatgctcttcactag

SEQ ID NO: 14 - DNA, 150 bp, synthetic construct
  source 1..150: mol_type = other DNA
gaagctacccctaacacactattctacacacagaaaatgctcttcactaggaagctacccctaacacactattctacacacagaaaatgctcttcactaggaagctacccctaacacactattctacacacagaaaatgctcttcactag

SEQ ID NO: 15 - DNA, 66 bp, synthetic construct
  source 1..66: mol_type = other DNA
gactacaaagaccatgacggagattataaagatcatgacatcgattacaaggatgacgatgacaag

SEQ ID NO: 16 - DNA, 30 bp, synthetic construct
  source 1..30: mol_type = other DNA
tccggactcagatctggaggctccggaggc

SEQ ID NO: 17 - DNA, 378 bp, synthetic construct
  source 1..378: mol_type = other DNA
ccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggagaggcttcccgcctggcgcattacaacaagcgctcgaccatcacctcccgggagatccagacggccgtgcgcctgctgctgcctggggagttggccaagcacgccgtgtccgagggtactaaggccatcaccaagtacaccagcgctaagtaa

SEQ ID NO: 18 - DNA, 246 bp, synthetic construct
  source 1..246: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 19 - DNA, 609 bp, synthetic construct
  source 1..609: mol_type = other DNA
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 20 - DNA, 204 bp, synthetic construct
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 21 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 22 - DNA, 78 bp, synthetic construct
  source 1..78: mol_type = other DNA
ggcagcggcgccaccaacttcagcctgctgaagcaggccggcgacgtggaggagaaccccggccccggagctagcgga

SEQ ID NO: 23 - protein, 21 aa, synthetic construct
GSGEGRGSLLTCGDVEENPGP

SEQ ID NO: 24 - protein, 24 aa, synthetic construct
GSGQCTNYALLKLAGDVESNPGPP

SEQ ID NO: 25 - protein, 25 aa, synthetic construct
GSGVKQTLNFDLLKLAGDVESNPGP

SEQ ID NO: 26 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 27 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 28 - DNA, 146 bp, synthetic construct
  source 1..146: mol_type = other DNA
catgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 29 - DNA, 149 bp, synthetic construct
  source 1..149: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctg

SEQ ID NO: 30 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - DNA, 747 bp, synthetic construct
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 33 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 34 - protein, 6 aa, synthetic construct
HHHHHH

SEQ ID NO: 35 - protein, 8 aa, synthetic construct
DYKDDDDK

SEQ ID NO: 36 - protein, 8 aa, synthetic construct
DLYDDDDK

SEQ ID NO: 37 - protein, 15 aa, synthetic construct
GLNDIFEAQKIEWHE

SEQ ID NO: 38 - protein, 26 aa, synthetic construct
KRRWKKNFIAVSAANRFKKISSSGAL

SEQ ID NO: 39 - protein, 9 aa, synthetic construct
YPYDVPDYA

SEQ ID NO: 40 - protein, 10 aa, synthetic construct
EQKLISEEDL

SEQ ID NO: 41 - protein, 8 aa, synthetic construct
WRHPQFGG

SEQ ID NO: 42 - protein, 8 aa, synthetic construct
WSHPQFEK

SEQ ID NO: 43 - protein, 13 aa, synthetic construct
SLAELLNAGLGGS

SEQ ID NO: 44 - protein, 8 aa, synthetic construct
TQDPSRVG

SEQ ID NO: 45 - protein, 14 aa, synthetic construct
GKPIPNPLLGLDST

SEQ ID NO: 46 - protein, 4 aa, synthetic construct
SAQA

SEQ ID NO: 47 - protein, 11 aa, synthetic construct
SDGTLAVPFKA

SEQ ID NO: 48 - protein, 7 aa, synthetic construct
NRGTEWD

SEQ ID NO: 49 - protein, 7 aa, synthetic construct
QAVRTSL

SEQ ID NO: 50 - protein, 7 aa, synthetic construct
TLAVPFK

SEQ ID NO: 51 - protein, 7 aa, synthetic construct
DSPAHPS

SEQ ID NO: 52 - DNA, 495 bp, synthetic construct
  source 1..495: mol_type = other DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga

SEQ ID NO: 53 - DNA, 588 bp, synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 54 - DNA, 348 bp, synthetic construct
  source 1..348: mol_type = other DNA
agagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctaga

SEQ ID NO: 55 - DNA, 537 bp, Homo sapiens
  source 1..537: mol_type = other DNA
gtagtctgcctcaggtacacactgagaaactgctttaatgtaacctgacccacggttattagtgaaaatatcacttttgttgttaccttattcccaacaaattcatttctgctttaatggaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcacaaatgtttaattaaaaaaaaaaaaagtcatgtactttcatagatattcacaatcctctctagtatactttcaaatcagttttaatttcagtttagtgtttttatgt

SEQ ID NO: 56 - DNA, 214 bp, synthetic construct
  source 1..214: mol_type = other DNA
aaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagct

SEQ ID NO: 57 - DNA, 306 bp, synthetic construct
  source 1..306: mol_type = other DNA
tgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataa

SEQ ID NO: 58 - DNA, 108 bp, synthetic construct
  source 1..108: mol_type = other DNA
atggtcaagatcatctccaggaagtctctgggtacacagaatgtctacgatatcggagtcgagaaagaccacaattttctcctgaaaaacggactcgtggcgtccaat

SEQ ID NO: 59 - DNA, 3278 bp, synthetic construct
  source 1..3278: mol_type = other DNA
atggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattca

SEQ ID NO: 60 - DNA, 2977 bp, synthetic construct
  source 1..2977: mol_type = other DNA
gcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaa

SEQ ID NO: 61 - DNA, 2999 bp, synthetic construct
  source 1..2999: mol_type = other DNA
atggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatg

SEQ ID NO: 62 - DNA, 3257 bp, synthetic construct
  source 1..3257: mol_type = other DNA
tgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaa

SEQ ID NO: 63 - DNA, 2972 bp, synthetic construct
  source 1..2972: mol_type = other DNA
atggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggac

SEQ ID NO: 64 - DNA, 3284 bp, synthetic construct
  source 1..3284: mol_type = other DNA
tgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaa

SEQ ID NO: 65 - DNA, 6022 bp, Homo sapiens
  source 1..6022: mol_type = genomic DNA
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaaggga

SEQ ID NO: 66 - DNA, 696 bp, synthetic construct
  source 1..696: mol_type = other DNA
atggtctccaaaggagaagcggtcattaaagagttcatgaggttcaaggttcatatggaaggctccatgaatggtcatgagttcgagattgaaggggagggtgaggggagaccttatgagggcactcagacagcgaaattgaaggtgacaaagggaggacctctcccgttcagttgggacatattgtcaccgcaatttatgtatggttctagagccttcactaagcaccctgccgacatcccagattactacaagcaatccttccctgagggctttaagtgggagagagtaatgaattttgaagatggcggggcagtcacagtaacacaagatacatccctggaagatggaacacttatctacaaagttaagctcagaggaacgaattttccaccggacggtccagtgatgcaaaaaaaaacaatgggttgggaagcatctacagagcgactgtaccctgaagacggtgtgctgaagggggacatcaaaatggccctgcgacttaaggatggagggcgctatttggcagatttcaagactacttacaaagccaaaaagcctgtacaaatgcctggagcttacaacgtggataggaagcttgatattaccagtcacaatgaagattatacagtggtagaacaatatgaacgctcagaaggtcgccacagcactggaggcatggatgagttgtacaag

SEQ ID NO: 67 - DNA, 84 bp, synthetic construct
  source 1..84: mol_type = other DNA
gatccaaagaagaaaaggaaagttgatcccaaaaagaagaggaaagtagatccaaaaaagaagcgaaaagtagggtacaagaag

SEQ ID NO: 68 - DNA, 66 bp, synthetic construct
  source 1..66: mol_type = other DNA
gactacaaagaccatgacggagactataaagatcatgacatcgattacaaggatgacgatgacaag

SEQ ID NO: 69 - DNA, 66 bp, synthetic construct
  source 1..66: mol_type = other DNA
tacccgtatgatgtcccggattacgctggcagctacccatacgatgtacccgactatgccggcagt

SEQ ID NO: 70 - DNA, 181 bp, synthetic construct
  source 1..181: mol_type = other DNA
tgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 71 - DNA, 50 bp, synthetic construct
  source 1..50: mol_type = other DNA
caataaaagatctttattttcattagatctgtgtgttggttttttgtgtg

SEQ ID NO: 72 - DNA, 586 bp, synthetic construct
  source 1..586: mol_type = other DNA
ccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 73 - DNA, 4586 bp, synthetic construct
  source 1..4586: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaacggaccgagcggccgc

SEQ ID NO: 74 - DNA, 4091 bp, synthetic construct
  source 1..4091: mol_type = other DNA
gcggccgcacgcgtggtaccctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggctaaataaagatggctttttagtattaaaagtggaagaaaattacaggtaattatctttgacggtaaaaacgctgtaatcagcgggctacatgaaaaattactctaattatggctgcatttaagagaatggagctcgggctgggcataaaagtcagggcagagccatctattgcttacatttgcttctgggatccaccatggtcaagatcatctccaggaagtctctgggtacacagaatgtctacgatatcggagtcgagaaagaccacaattttctcctgaaaaacggactcgtggcgtccaattgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaacggaccgagcggccgc

SEQ ID NO: 75 - DNA, 4528 bp, synthetic construct
  source 1..4528: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 76 - DNA, 4033 bp, synthetic construct
  source 1..4033: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtcaagatcatctccaggaagtctctgggtacacagaatgtctacgatatcggagtcgagaaagaccacaattttctcctgaaaaacggactcgtggcgtccaattgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 77 - DNA, 5956 bp, synthetic construct
  source 1..5956: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgaaggcctccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacccgcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 78 - DNA, 5487 bp, synthetic construct
  source 1..5487: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggtcaagatcatctccaggaagtctctgggtacacagaatgtctacgatatcggagtcgagaaagaccacaattttctcctgaaaaacggactcgtggcgtccaattgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgagcccgggcagttccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacccgcggccgccaccatggtctccaaaggagaagcggtcattaaagagttcatgaggttcaaggttcatatggaaggctccatgaatggtcatgagttcgagattgaaggggagggtgaggggagaccttatgagggcactcagacagcgaaattgaaggtgacaaagggaggacctctcccgttcagttgggacatattgtcaccgcaatttatgtatggttctagagccttcactaagcaccctgccgacatcccagattactacaagcaatccttccctgagggctttaagtgggagagagtaatgaattttgaagatggcggggcagtcacagtaacacaagatacatccctggaagatggaacacttatctacaaagttaagctcagaggaacgaattttccaccggacggtccagtgatgcaaaaaaaaacaatgggttgggaagcatctacagagcgactgtaccctgaagacggtgtgctgaagggggacatcaaaatggccctgcgacttaaggatggagggcgctatttggcagatttcaagactacttacaaagccaaaaagcctgtacaaatgcctggagcttacaacgtggataggaagcttgatattaccagtcacaatgaagattatacagtggtagaacaatatgaacgctcagaaggtcgccacagcactggaggcatggatgagttgtacaagagcgctgatccaaagaagaaaaggaaagttgatcccaaaaagaagaggaaagtagatccaaaaaagaagcgaaaagtagggtacaagaagtgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 79 - DNA, 4583 bp, synthetic construct
  source 1..4583: mol_type = other DNA
gcggccgcacgcgtagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagatcgactggatccttcgaagctagcgctgccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaataaaagatctttattttcattagatctgtgtgttggttttttgtgtgcggaccgagcggccgc

SEQ ID NO: 80 - DNA, 4088 bp, synthetic construct
  source 1..4088: mol_type = other DNA
gcggccgcacgcgtagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagatcgactggatccttcgaagctagcgctgccaccatggtcaagatcatctccaggaagtctctgggtacacagaatgtctacgatatcggagtcgagaaagaccacaattttctcctgaaaaacggactcgtggcgtccaattgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggaataaaagatctttattttcattagatctgtgtgttggttttttgtgtgcggaccgagcggccgc

SEQ ID NO: 81 - DNA, 4790 bp, synthetic construct
  source 1..4790: mol_type = other DNA
gcggccgcacgcgtgaaatcataaatgctgagggtagtctgcctcaggtacacactgagaaactgctttaatgtaacctgacccacggttattagtgaaaatatcacttttgttgttaccttattcccaacaaattcatttctgctttaatggaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcacaaatgtttaattaaaaaaaaaaaaagtcatgtactttcatagatattcacaatcctctctagtatactttcaaatcagttttaatttcagtttagtgtttttatgttttgtgaagatacgcgagctcgggctgggcataaaagtcagggcagagccatctattgcttacatttgcttctgggatccagatctttcgaagctagcgctaccggtcgccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaacggaccgagcggccgc

SEQ ID NO: 82 - DNA, 4295 bp, synthetic construct
  source 1..4295: mol_type = other DNA
gcggccgcacgcgtgaaatcataaatgctgagggtagtctgcctcaggtacacactgagaaactgctttaatgtaacctgacccacggttattagtgaaaatatcacttttgttgttaccttattcccaacaaattcatttctgctttaatggaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcacaaatgtttaattaaaaaaaaaaaaagtcatgtactttcatagatattcacaatcctctctagtatactttcaaatcagttttaatttcagtttagtgtttttatgttttgtgaagatacgcgagctcgggctgggcataaaagtcagggcagagccatctattgcttacatttgcttctgggatccagatctttcgaagctagcgctaccggtcgccaccatggtcaagatcatctccaggaagtctctgggtacacagaatgtctacgatatcggagtcgagaaagaccacaattttctcctgaaaaacggactcgtggcgtccaattgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaacggaccgagcggccgc

SEQ ID NO: 83 - DNA, 4249 bp, synthetic construct
  source 1..4249: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtatggaagtagctgggcaggcgatgtgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 84 - DNA, 4312 bp, synthetic construct
  source 1..4312: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtcaagatcatctccaggaagtctctgggtacacagaatgtctacgatatcggagtcgagaaagaccacaattttctcctgaaaaacggactcgtggcgtccaattgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 85 - DNA, 4222 bp, synthetic construct
  source 1..4222: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtctacccgtatgatgtcccggattacgctggcagctacccatacgatgtacccgactatgccggcagtatggagcaaacagttttggtccctccgggaccagacagtttcaatttctttactcgggagagtcttgccgccattgagaggcgcatagctgaggtaagtactagcagctacaatccagctaccattctgcttttattctatggttgggataaggctggattattctgagtccaagctaggcccttttgctaatcatgttcatacctcttatcttcctcccacaggaaaaggctaagaatccaaaacctgacaagaaagacgacgacgaaaacggacccaaacctaactcagatctcgaagctggaaagaatctcccattcatctatggtgatatccctccagaaatggtttcagaacctctagaagatctcgatccatactatatcaataaaaagaccttcatcgttctgaacaaaggaaaggcgattttccggttttctgctacttctgctctctatattctcacaccatttaatccacttcgcaagattgcgattaagatactggtgcatagtctgttcagtatgctgattatgtgtacaatccttacaaattgtgtctttatgactatgtctaacccgccggattggaccaagaacgtagaatacacgttcactggaatctatacgttcgagtctcttattaagataatcgccagggggttctgtcttgaggatttcactttcctccgcgatccgtggaattggcttgacttcaccgttattacgttcgcttacgttactgagttcgttgatcttggcaacgtgtctgcactcagaacattcagagtgcttagagcacttaaaaccataagtgtcataccaggattgaaaacgatcgtgggagctctgatacagagtgtaaagaagctttcagatgtaatgatccttactgtcttctgtctttccgtattcgcactcatcgggctccagctgtttatgggtaacctcagaaacaaatgcattcaatggccaccaacaaatgcgagccttgaggaacatagcatagaaaagaatatcactgttaactataatgggaccctcataaacgaaaccgtgttcgaatttgactggaaatcctacattcaggattccagatatcattattttcttgagggcttcttggacgcacttttgtgcggaaattcaagtgatgctggtcaatgtcctgaaggttatatgtgtgttaaagcaggaagaaacccaaactacggatacacatctttcgatacattttcttgggctttcctatctctttttcggcttatgacacaagacttttgggaaaatttgtatcagctgacactccgagcggctggaaaaacttatatgatcttcttcgttcttgtaatctttttgggatccttctacctcatcaatttgatacttgcagttgtcgctatggcatacgaggagcaaaatcaagcaacgctagaagaagcggagcagaaagaggctgaatttcaacagatgattgagcaattgaagaaacaacaggaagctgcacaacaagcagctactgctactgcatctgaacattctagagagccaagtgcagctggaaggctttctgatagttcaagtgaagcatctaaattgagttctaagtcagcaaaggaacggagaaatagacggaaaaaacgaaagcagaaggagcaatctggaggagaagagaaggacgaagacgagtttcaaaaaagtgaatcagaggactcaattagacgcaaaggattcagatttagtatcgaaggaaatagattgacttatgaaaaacgatattcctcaccacatcagtcactcctgagtatacgcgggtcactcttttcaccgagacgaaattccagaacttcactcttctcattccggggaagggctaaggatgtaggctcagaaaatgatttcgcagacgatgagcattccacttttgaagataatgagagcaggcgagacagtctctttgtaccacgaagacatggcgaaagaaggaacagcaaccttagccagactagtcggtccagtagaatgctagctgtattccctgctaatggcaagatgcattccaccgttgattgtaatggggtcgtctcgttagtaggtggaccttcagttcctacctcaccggttggacaattgctgccggagggaaccactactgagactgaaatgagaaaacgacgttcttcaagcttccatgtgtctatggattttttggaagacccgtcacagcgccaaagagctatgtctatagcttcaatcctgacaaacaccgtagaggagttggaggagtcacgccagaagtgccctccttgttggtacaagttctccaacatcttcctgatttgggattgttcaccttattggctgaaagtcaagcacgttgttaacctcgtcgtaatggatccttttgtcgacctggctataacgatatgtatcgtcctgaacacactcttcatggctatggagcattatccgatgactgatcattttaacaatgtgcttaccgtgggtaatctggttttcactggcatctttactgcagaaatgtttcttaagattattgcaatggacccctactactactttcaagaaggatggaatatttttgatggttttatcgtcacactttctttggttgaattgggcttggcaaatgtagaggggctctcagttcttagaagtttcagacttctccgggtattcaagcttgctaagagctggcctactttgaacatgctcatcaagattatcggaaacagtgttggcgcccttggcaatctgacattggtgttggctatcatagtattcatcttcgcggttgtgggaatgcagttgtttgggaagtcatacaaggactgtgtgtgcaagatagcgtccgactgtcaacttccgaggtggcacatgaacgatttctttcattcattcctcattgtgtttcgggtcctctgtggcgagtggatagaaactatgtgggactgtctcagttatgacacagaaatcttgacggtggaatacgggtttcttccgatcggaaagattgttgaggagcgcatagagtgtacggtgtataccgtcgataagaacggatttgtctacacacagcctatcgcacaatggcataatagaggagaacaagaagtcttcgaatattgtttggaggacggatcaatcatacgggcaaccaaagaccacaagtttatgacaacagatggacagatgttgccaatagatgagatatttgagaggggacttgatctcaagcaagtggatggtctgccataatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 86 - DNA, 4339 bp, synthetic construct
  source 1..4339: mol_type = other DNA
gcggccgcacgcgtttaattaagtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaaccggctagaggatccttcgaagctagcgctaccggtgccaccatggtcaagatcatctccaggaagtctctgggtacacagaatgtctacgatatcggagtcgagaaagaccacaattttctcctgaaaaacggactcgtggcgtccaattgtatggaagtagctgggcaggcgatgtgccttacggtattcatgatggtcatggtcatcggaaatcttgttgtattgaatttatttctcgcgttgttgttgagttcattttccgccgataatttggctgccactgatgacgacaacgagatgaataatcttcagatagctgtagaccggatgcacaaaggcgttgcctacgtcaaacgaaaaatctatgaattcatacagcaatccttcatacgaaaacagaagattctggatgaaatcaaaccccttgatgatctcaataataagaaagattcatgcatgtcgaaccataccacagagataggcaaggaccttgactaccttaaagacgtgaacggtaccacaagtggaataggcacaggtaagtactagcagctacaatccagctaccattctgcttttattctatggttgggataaggctggattattctgagtccaagctaggcccttttgctaatcatgttcatacctcttatcttcctcccacaggctcctctgtagagaagtacatcatagacgagagcgattacatgtctttcatcaacaacccgtccctcactgtcaccgttcccatcgccgtaggagaatctgacttcgagaatctcaatacggaggatttcagctccgaatcagacttggaggaatcaaaggagaagttgaacgaaagttcaagttcatccgagggcagcaccgtggacataggcgcccccgtcgaggaacaacctgtagtcgagcctgaggaaactttggaacccgaagcgtgtttcacggaggggtgtgttcaacgcttcaagtgttgccaaattaacgttgaagagggtcgtggaaaacaatggtggaacctccgcaggacctgtttccggatcgtcgaacataattggttcgagacgttcatagttttcatgatcttgctttcatctggtgctttggcattcgaggatatctacatcgaccaacgaaagaccataaaaactatgctggaatatgcagacaaggttttcacatacatattcatccttgaaatgctcctgaaatgggtagcgtatggttaccagacttatttcacgaacgcatggtgctggctcgatttcctgattgtcgacgtctccctggtgtcattgactgctaacgcactcggatatagcgaactaggcgctattaagagtctcagaaccctgagagcattgaggcccctccgcgcgctctctcggtttgagggaatgagagtagtcgttaatgcactgttgggagcgataccttccattatgaacgtgcttctcgtttgtctcatcttctggctgatattctctattatgggtgtgaacttgttcgcaggcaaattttaccactgcattaacacaactacaggagatagatttgatattgaggatgtaaacaaccacaccgactgtttgaagttgatagagagaaacgagaccgcaagatggaagaatgtaaaagtcaacttcgacaatgtcggctttggatatctttcactgctgcaagtagccacattcaaaggatggatggacattatgtacgctgcagtagattcccgaaacgtagagttgcaaccgaagtatgaagaaagtttgtatatgtacctctacttcgtaatttttatcatctttggctcattcttcacacttaacctgttcattggtgtaatcatcgacaatttcaatcagcagaaaaagaaatttggtggacaagacatcttcatgacagaggaacagaagaaatactataatgcaatgaaaaaactagggtccaaaaagccccaaaaacctattcctagaccgggcaacaagtttcaaggcatggttttcgacttcgtaactagacaggtgtttgatatatctattatgattctgatatgtctgaatatggttacgatgatggttgagactgatgatcaatctgaatacgttacgacgatacttagccgaattaacttggtattcattgttcttttcacgggcgaatgtgtacttaaactgattagtttaaggcactattatttcacaatcggttggaacatttttgatttcgttgtggtcatactttccattgttggcatgtttcttgctgaattgatagaaaagtacttcgtcagtccaacacttttccgagttatacggcttgcccgaatcggacgaattctcaggctaatcaaaggtgctaaaggaattcgtacactgcttttcgctctcatgatgtcactgccagctcttttcaacatcggtttgttactatttttggtaatgtttatatatgcgatcttcggcatgagtaatttcgcttatgttaaacgggaggtgggaatcgatgacatgtttaattttgagacattcggcaattctatgatctgtctctttcaaattaccacgtcagctggatgggacggattgcttgctccgattctcaacagtaaaccgcccgattgcgaccctaacaaagtgaatccgggttcatctgtaaagggagactgcggaaatccgagcgtcggtatcttctttttcgtctcctacattataatttctttccttgttgtcgtgaacatgtatatagctgtgatcttggaaaatttttctgttgctactgaggaatccgcagaaccactttcagaagacgattttgagatgttttacgaagtttgggagaagtttgatcctgacgctacacagtttatggaatttgagaagctctcacagttcgcagctgccctggagcctccgttgaatcttccacagcctaacaagttacaactgattgcgatggacctgccaatggtgtctggggaccgaatccactgccttgatatactctttgctttcacaaaaagggtcttgggcgagtctggagaaatggacgccctcagaatacagatggaggaacgattcatggcttcgaatcctagcaaagtgtcttatcaacccatcactacgactcttaaaagaaaacaagaggaagtgtctgctgtcattatccagcgagcatatagacggcacttgctcaaacgaactgttaagcaagccagtttcacctacaataaaaacaaaataaaaggtggtgctaatttgctgattaaagaggacatgattatcgacagaatcaatgagaactccattacagaaaaaaccgatctcactatgtcaacagcagcctgtcctccctcatacgaccgtgtcactaaacctatagtcgaaaaacatgaacaagagggcaaggatgagaaggccaaaggcaaagccggcgactacaaagaccatgacggagactataaagatcatgacatcgattacaaggatgacgatgacaagtaatgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggcaataaaagatctttattttcattagatctgtgtgttggttttttgtgtggtgcggaccgagcggccgc

SEQ ID NO: 87 - DNA, 450 bp, synthetic construct
  source 1..450: mol_type = other DNA
gcggccttaaagagaccggttcactgtgacagtaaaagagaccggttcactgtgagaatgaaagagaccggttcactgtgatcggaaaagagaccggttcactgtgagcggccttaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctgcggccttgaaacccagcagacaatgtagctcagtagaaacccagcagacaatgtagctgaatggaaacccagcagacaatgtagcttcggagaaacccagcagacaatgtagctgcggcc

(19) United States
(12) Patent Application Publication
(10) Pub. No.: US 2026/0224749 A1
(43) Pub. Date: Aug. 6, 2026

(54) INTEIN-MEDIATED RECONSTITUTION OF VOLTAGE-GATED SODIUM CHANNEL FUNCTION

(71) Applicants: Allen Institute, Seattle, WA (US); Seattle Children’s Hospital d/b/a Seattle Children’s Research Institute, Seattle, WA (US)

(72) Inventors: Bryan Gore, Redmond, WA (US); Edward Sebastian Lein, Mercer Island, WA (US); Boaz P. Levi, Seattle, WA (US); Refugio Martinez, Seattle, WA (US); John K. Mich, Seattle, WA (US); Franck Kalume, Bothell, WA (US)

(73) Assignees: Allen Institute, Seattle, WA (US); Seattle Children’s Hospital d/b/a Seattle Children’s Research Institute, Seattle, WA (US)

(21) Appl. No.: 19/153,092

(22) PCT Filed: Feb. 1, 2024
(86) PCT No.: PCT/US2024/014080

§ 371 Date: Aug. 1, 2025

Related U.S. Application Data
(60) Provisional application No. 63/482,741, filed on Feb. 1, 2023.

(51) Int. Cl. A61K 48/00; A61K 9/00; A61K 38/00; A61P 25/08; C07K 14/005; C07K 14/47; C12N 5/0793; C12N 15/86

(52) U.S. Cl. CPC A61K 48/0058; A61K 9/0085; A61K 48/0075; A61K 48/0083; A61P 25/08; C07K 14/005; C07K 14/47; C12N 5/0619; C12N 15/86; A61K 38/00; C07K 2319/92; C12N 2510/00; C12N 2750/14143; C12N 2750/14145; C12N 2800/40; C12N 2830/42; C12N 2830/48; C12N 2830/50; C12N 2840/203

(57) ABSTRACT

Artificial expression constructs for the rescue of voltage-gated sodium channel function using intein-mediated reconstitution of the SCN1A encoded protein, voltage-gated sodium channel alpha subunit 1 (Nav1.1) are described. Rescued voltage-gated sodium channel function can be used to treat disorders such as epilepsy, and more particularly, Dravet Syndrome.

### CROSS-REFERENCE TO RELATED APPLICATIONS

This application is a U.S. National Phase patent application based on International Patent Application No. PCT/US2024/014080, filed on Feb. 1, 2024, which claims priority to U.S. Provisional Patent Application No. 63/482,741 filed Feb. 1, 2023, each of which is incorporated herein by reference in its entirety as if fully set forth herein.

### STATEMENT REGARDING FEDERALLY SPONSORED RESEARCH OR DEVELOPMENT

This invention was made with government support under MH120095 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3GP5194.XML. The file is 180,224 bytes, was created Jul. 25, 2025, and is being submitted electronically via patent Center.

### FIELD OF THE DISCLOSURE

The current disclosure describes the rescue of voltage-gated sodium channel function using intein-mediated reconstitution of the SCN1A encoded protein, voltage-gated sodium channel alpha subunit 1 (Nav1.1). Rescued voltage-gated sodium channel function can be used to treat disorders such as epilepsy, and more particularly, Dravet Syndrome.

### BACKGROUND OF THE DISCLOSURE

There are numerous neurological disorders for which treatments are urgently needed. One class of such disorders arises from mutations in SCN1A, the gene that encodes the alpha subunit of voltage-gated sodium channel Nav1.1. For example, SCN1A mutation can cause disorders such as Dravet syndrome, myoclonic seizures, and intractable childhood epilepsy with generalized tonic-clonic seizures.

Epilepsy is a neurological disorder that occurs when the brain presents an enduring predisposition to generate two or more epileptic seizures. An epileptic seizure is a temporary disruption of brain function due to abnormal excessive or synchronous neuronal activity. Its manifestation may include periods of unusual behavior, sensations and sometimes loss of consciousness.

Dravet Syndrome (DS) particularly is a rare and catastrophic form of intractable epilepsy that begins in infancy. Initially, the patient experiences prolonged seizures. In their second year, additional types of seizures begin to occur and this typically coincides with a developmental decline. This leads to poor development of language and motor skills.

Children with DS are likely to experience multiple seizures per day. Epileptic seizures are far more likely to result in death in sufferers of DS; 10 to 16% of patients diagnosed with DS die in childhood, particularly between two and four years of age. Additionally, patients are at risk of numerous associated conditions including orthopedic developmental issues, impaired growth, sleep and circadian rhythm impairments, and chronic infections.

Of particular concern, children with DS are particularly susceptible to episodes of Status epilepticus. Status epilepticus is a condition in which a seizure lasts for more than 5 minutes or multiple seizures occur close together within a 5 minute-period without recovery of consciousness between them. This severe condition is categorized as a medical emergency requiring immediate medical intervention, typically involving hospitalization. Prolonged convulsive status epilepticus lasting >30 min can be fatal and lead to substantial brain damage. Frequent hospitalizations of children with DS are distressing for the patient, family and caregivers. The cost of care for DS is also high as the affected children require constant supervision and many require institutionalization.

At present, although a number of anticonvulsant therapies can be employed to reduce the instance of seizures in patients with DS, the results obtained with such therapies are typically poor and those therapies only produce partial cessation of seizures in most patients. Many of these anticonvulsants such as clobazam and clonazepam have undesirable side effects, which are particularly acute in pediatric patients. Furthermore, certain anticonvulsants (particularly the sodium-channel blockers) exacerbate the seizures in DS patients.

Cell-type or cell-class specific gene delivery using non-pathogenic viral delivery is showing increasing promise for the treatment of diverse diseases. Inclusion of gene regulatory elements, such as specific promoters or enhancers, within the delivered vector, has been beneficial to provide specificity for gene expression within particular targeted cell types. For example, Dimidschstein and colleagues (Nat Neurosci. 19(12):1743-1749, 2016) developed a viral delivery gene construct based on the adeno-associated virus (AAV) that resulted in selective expression of a gene within gamma-aminobutyric acid (GABA)ergic interneurons within the telencephalon, a cell type important in the treatment of epilepsy.

One significant drawback to using AAV as a selective gene-delivery system is the strictly restricted packaging limit of AAVs; this is particularly limiting to the large 6 kb SCN1A gene which exceeds the packaging capacity of AAV (4.7 kb DNA).

### SUMMARY OF THE DISCLOSURE

The current disclosure provides expression constructs that result in unexpectedly rapid and high levels of protein expression within targeted central nervous system cell types for the purpose of rescuing voltage-gated sodium channel function. The expression constructs can be used to reverse or ameliorate the effects of voltage-gated sodium channel dysfunction in targeted cell types due to SCN1A variants that cause a disorder. Typically, SCN1A is difficult to deliver because of its large size which is greater than the packaging capacity of an adeno-associated virus (AAV). As described herein, the SCN1A coding sequence is thus packaged into two or more artificial expression constructs with sequences encoding a split intein N- and C-fragments, wherein the expression product of the two or more artificial expression constructs are spliced together to result in full-length SCN1A protein with the intein fragments spliced out.

In particular embodiments, the current disclosure provides systems and methods to express the SCN1A coding sequence in a subject in need thereof. In particular embodiments, the system includes at least a first and a second artificial expression construct, wherein the first artificial expression construct includes a first portion of the SCN1A coding sequence, an N-intein coding sequence, and a first promoter sequence, wherein the N-intein coding sequence is located at the 3′ end of the first portion of the SCN1A coding sequence; and the second artificial expression construct includes a second portion of the SCN1A coding sequence, a C-intein coding sequence, and a second promoter sequence, wherein the C-intein coding sequence is located at the 5′ end of the second portion of the SCN1A coding sequence. In particular embodiments, when the first artificial expression construct and the second artificial expression construct are both expressed in a cell, the protein product of the SCN1A coding sequence is produced by protein splicing. In particular embodiments, the first and/or second artificial expression construct includes an enhancer that leads to targeted expression of the first and/or second portion of the SCN1A coding sequence within a targeted central nervous system cell population.

In particular embodiments, the current disclosure provides treatment of SCN1A-related disorders such as Dravet syndrome, myoclonic seizures, and intractable childhood epilepsy with generalized tonic-clonic seizures. For example, as disclosed herein, administration of the artificial expression constructs results in therapeutic efficacy to treat thermally-induced seizures, myoclonic seizures, and generalized tonic-clonic seizures in a well-established in vivo mouse model of the disease.

In particular embodiments, the treatment includes the administration of at least a first and a second artificial expression construct to express the SCN1A coding sequence in a subject in need thereof. In particular embodiments, the first and second artificial expression construct each include a portion of the SCN1A coding sequence, a split intein coding sequence, and a promoter sequence.

Particular embodiments use split inteins including consensus fast intein (Cfa)-N and Cfa-C.

In particular embodiments, the first portion of the SCN1A coding sequence and the second portion of the SCN1A coding sequence, when expressed and spliced together form a mature protein that rescues voltage-gated sodium channel function in a cell. The artificial expression construct can also include other regulatory elements if necessary or beneficial.

In particular embodiments, the artificial expression constructs are expressed (e.g., after transduction) in all neurons. In particular embodiments, the artificial expression constructs include an hSyn1 promoter and are expressed (e.g., after transduction) in neurons. In particular embodiments, the artificial expression constructs are expressed in all cell populations. In particular embodiments, the artificial expression constructs include a CMV promoter and are expressed in cell populations.

In particular embodiments, an enhancer is used to drive gene expression in a targeted central nervous system cell population. Particular embodiments of the artificial expression constructs utilize the following enhancers to drive gene expression within targeted central nervous system cell populations as follows (enhancer/targeted cell population): DLX2.0/forebrain GABAergic; hSyn1, and 4×2C or 8×2C miR binding site/pan-GABAergic neurons; eHGT_078h/forebrain glutamatergic neurons. In particular embodiments, the artificial expression construct can include a shortened promoter or a minimal promoter. In particular embodiments, the shortened promoter includes the shortened hSyn1 promoter. In particular embodiments, the minimal promoter includes minBglobin.

Particular embodiments provide artificial expression constructs including the features of vector pairs described herein including vectors: CN3252 and CN3254, CN3683 and CN3684, CN3251 and CN3253, CN3677 and CN3678, CN4541 and CN4542, CN4217 and CN4218, or CN4642 and CN4643.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D. Efficient intein-mediated reconstitution of human SCN1A from split halves. (1A) Design of split NaV1.1 halves with intein fusion protein tags (Cfa-N and Cfa-C). Full length human SCN1A (hSCN1A) encoding NaV1.1 (1998 amino acids) is split into two halves (N-terminal 1-1049 amino acids, and C-terminal 1050-1998 amino acids) that can be packaged into AAV particles. The breakpoint is at an endogenous cysteine residue (Cys1050), which enables the protein joining to be scarless. Abbreviations: Cfa, Consensus Fast intein, Stevens et al., J Am Chem Soc. 138(7):2162-5, 2016. (1B) Plasmid vectors for testing the fusion protein in cell lines. HA tag is added to the N-terminus of the N-terminal half, and FLAG to the C-terminus of the C-terminal half. Also, SYFP2 and mScarlet reporters are added for transfection tracking. Abbreviations: CMV, cytomegalovirus immediate/early promoter; IRES2, internal ribosome entry site 2. (1C) Western blot demonstrates efficient joining of the two halves in HEK-293 cells. HEK-293 cells were transfected with the indicated plasmid vectors using polyethyleneimine, and prepared protein lysates for western blotting after 72 hours. Note that full length hSCN1A runs near expected size of 250 kDa, and the N- and C-terminals run near their expected sizes of 134 and 115 kDa, respectively. Lanes 5 and 6 in the anti-FLAG blot are inexplicably dirty, marked by asterisks, but nevertheless the expected protein sizes can be seen (all bands at expected sizes marked by arrows). (1D) Electrophysiology demonstrates functional voltage-gated sodium channel activity from split SCN1A channels in CHO cells. CHO cells were transfected with the indicated plasmid vectors using polyethyleneimine, and then cells were patch clamped in voltage-clamp mode and subjected to stepwise depolarization conditions while measuring current flow. Maximal sodium current flow (Ipeak) is shown normalized to cell capacitance which is driven by cell size. Both full length and intein-fusion hSCN1A conditions yield a high level of current, but control conditions give low levels of current.

FIGS. 2A-2F. Cell type-specific delivery of assembled hSCN1A from AAVs in mouse brain. (2A) Design of an AAV vector system for delivery of SCN1A using split SCN1A halves with intein fusion protein tags and inhibitory neuron-specific enhancer DLX2.0 and minimal promoter (minP) driving each half. Abbreviations: WPRE3 woodchuck hepatitis virus post-translational regulatory element 3, pA polyA site, LITR left ITR, RITR right ITR. (2B) Intein-mediated production of full-length hSCN1A in mouse brain from AAV vectors. The indicated AAVs packaged in PHP.eB were delivered intravenously to mice (3×10¹¹ genome copies [GC] each AAV vector IV at adult stage). After three weeks brain was extracted and cell membrane protein lysate was prepared from primary visual cortex for western blot. N and C terminal vectors produced full length SCN1A (NaV1.1) protein, only when supplied together. The single halves were not detected alone, possibly from poor enrichment using membrane protein lysates that might not purify the unjoined halves, or low protein half-life. (2C) Specific detection of each hSCN1A half in mouse brain by immunohistochemistry (IHC). The indicated AAV/PHP.eB vectors were administered at the neonatal stage via the intracerebroventricular route (ICV, 3×10¹⁰ GC each). After three weeks brain was extracted and expression pattern of each half was assessed by Immunohistochemistry (here shown in primary visual cortex, VISp). Each half can be detected singly or together, and they are found in cell somata and processes of scattered neurons (presumptive inhibitory neurons). (2D) Expression of assembled hSCN1A was seen in scattered neurons throughout the forebrain (presumptive interneurons). The N-term and C-term vectors were delivered by ICV injections (3×10¹⁰ GC each), and analyzed expression at adult stage by IHC. (2E) Costaining assembled hSCN1A with Gad67, a marker of interneurons, suggests that many interneurons express assembled hSCN1A in VISp. The same mouse is shown in FIG. 2D. (2F) Quantitative biodistribution analysis in VISp Gad67+ neurons suggests that expression is highly specific to inhibitory neurons (defined as percent of Marker+ cells that are also Gad67+, typically 95-100% across layers of cortex), and many VISp Gad67+ neurons express the HA and FLAG markers. The completeness of labeling (defined as percent of Marker+/Gad67+ cells out of all Gad67+ cells) is dose-dependent, with more neurons expressing at the 3×10¹⁰ GC dose than at the 1×10¹⁰ GC dose, and also higher with more highly purified AAV vector preps produced at Packgene than those produced at the Allen Institute viral core. Mouse K9263 is shown in FIGS. 2D and 2E for reference.

FIG. 3. In vivo testing of hSCN1A split intein AAVs in Dravet syndrome mice. Diagram shows the experimental set-up to test the function of DLX2.0-hSCN1A split intein vectors. Dravet syndrome (DS) mutant or control animals were left uninjected or were injected with front half and back half DLX2.0 split intein SCN1A fusion vectors packaged in PHP.eB serotype at a low dose (1×10¹⁰ GC per vector) or high dose (3×10¹⁰ GC per vector). In the high dose group, vectors from two sources were tested: in-house packaged virus, or obtained from a commercial source (PackGene). Neonatal mice were injected with virus between P0 and P3. They were then monitored until postnatal day 70 (P70) for survival.

FIG. 4. DLX2.0-SCN1A split intein vectors completely rescue mortality in Dravet syndrome (DS) model. Kaplan-Meier curves show low and high dosing regimens all provided rescue of mortality in DS mice, and the Packgene sourced virus completely rescued of early mortality.

FIGS. 5A-5C. DLX2.0-Split Intein SCN1A vectors provided substantial rescue of heat-induced seizures. (5A, 5B) Both in-house (IH) produced virus, and commercially-produced (PG) virus showed a substantial resistance to heat-induced myoclonic seizures, with a nearly two degree shift in the temperature of myoclonic seizure onset (5A) and a significantly reduced seizure burden (cumulative number of seizures) (5B). (5C) Both in-house (IH) produced virus, and commercially-produced (PG) virus show an almost complete rescue heat-induced generalized tonic clonic seizures.

FIGS. 6A, 6B. hSyn1-hSCN1A split intein vectors, unlike DLX2.0-hSCN1A split intein vectors, showed toxicity in juvenile mice. (6A) After injection at the neonatal stage, both high dose (3×10¹⁰) and low dose (1×10¹⁰) hSyn1-hSCN1A split intein vectors showed mortality, while none was observed with the DLX2.0-hSCN1A split intein vectors. This shows the pan-neuronal expression of with the hSyn1 promoter leads to early mortality. (6B) Both high dose and low dose hSyn1-hSCN1A vectors showed some continued mortality in DS mutant mice after weaning at the third week of life. Low and high dose vectors are compared with uninjected controls, or DS mice injected with control vector PHP.eB-CN1390: DLX2.0-SYFP2.

FIGS. 7A, 7B: High dose but not low dose hSyn1-hSCN1A split intein vectors can rescue heat-induced seizures. Dravet syndrome model mice were left untreated or were treated with various hSCN1A split intein vectors at the neonatal stage. After P70, surviving Dravet mice underwent heat induction to 42 degrees and were monitored for (7A) myoclonic, or (7B) generalized tonic clonic seizures. Note that the high dose of DLX2.0-hSCN1A substantially reduced myoclonic and generalized tonic clonic seizures without juvenile toxicity (FIGS. 6A, 6B). The high dose of pan-neuronal hSyn1-hSCN1A split intein reduced seizures, but caused significant toxicity (FIGS. 6A, 6B), while the low dose of pan-neuronal hSyn1-hSCN1A split intein did not reduce seizures compared to untreated mice.

FIGS. 8A-8D. High dose DLX2.0-hSCN1A split intein vectors completely rescued mortality and seizures in the Dlx5/6-Cre; Scn1a^{fl/+} Dravet model. (8A) Mating schematic of the Dlx5/6-Cre; Scn1a^{fl/+} model. Resulting pups of this mating scheme were tested. This model deletes one copy of Scn1a, only in forebrain GABAergic cells. These are the same cells in which DLX2.0 to drives expression. (8B) Kaplan-Meier curve shows that all untreated Dlx5/6-Cre; Scn1a^{fl/+} mutant mice died by the fourth week of life. In contrast, none of the mice injected with the DLX2.0-hSCN1A split intein vectors died after 10 weeks of life. (8C) After P70, mice were tested for heat-induced seizures and no mice showed myoclonic seizures (n=7). (8D) After P70, mice were tested for heat-induced seizures and no mice showed generalized tonic clonic seizures (n=7).

FIG. 9. Alternative sites for splitting human SCN1A to make AAV-sized halves. (9A) Design of the split protein at breakpoint Cys1050. This design was used in FIGS. 1-8. Note that an endogenous cysteine residue is required to make half joining scarless and reconstitute a full-length unmutated protein. (9B, 9C) Alternative designs of split intein breakpoints at either Cys957 (9B) or Cys948 (9C). These breakpoints would permit a better AAV size and packaging efficiency of the N-terminal half for better expression than that seen with hSCN1A-CO-Nterm1049 when using the Cys1050 breakpoint. However, they would place the intein junctions in the extracellular/lumenal space.

FIG. 10. Sequences supporting the disclosure include 1×hI56i(core) (core of human I56i enhancer) (SEQ ID NO: 1), DLX2.0 (3× human I56i core) (SEQ ID NO: 2), eHGT_078h (SEQ ID NO: 55), hSyn1 promoter (SEQ ID NO: 52), CMV promoter (SEQ ID NO: 53), shortened hSyn1 promoter (SEQ ID NO: 54), 4×2C (SEQ ID NO: 56), 8×2C (SEQ ID NO: 87), Cfa-N(SEQ ID NO: 57), Cfa-C(SEQ ID NO: 58), hSCN1A-CO-Nterm1049-Intron (SEQ ID NO: 59), hSCN1A-CO-Cterm949-Intron (SEQ ID NO: 60), hSCN1A-CO-Nterm956-Intron (SEQ ID NO: 61), hSCN1A-CO-Cterm1042-Intron (SEQ ID NO: 62), hSCN1A-CO-Nterm947-Intron (SEQ ID NO: 63), hSCN1A-CO-Cterm1051-Intron (SEQ ID NO: 64), Human SCN1A (SEQ ID NO: 65), Beta-Globin Minimal Promoter (pBGmin/minBGlobin/minBGprom) (SEQ ID NO: 3), minCMV Promoter (SEQ ID NO: 4), Mutated minCMV Promoter (SacI RE site removed) (SEQ ID NO: 5), minRho Promoter (SEQ ID NO: 6), minRho* Promoter (SEQ ID NO: 7), Hsp68 minimal Promoter (proHsp68) (SEQ ID NO: 8), SYFP2 (SEQ ID NO: 9), EGFP (SEQ ID NO: 10), mScarlet: (SEQ ID NO: 66), 3×NLS (SEQ ID NO: 67), Optimized Flp recombinase (FlpO) (SEQ ID NO: 11), Improved Cre recombinase (iCre) (SEQ ID NO: 12), SP10 insulator (SP10ins) (SEQ ID NO: 13), 3×SP10ins (SEQ ID NO: 14), 3×FLAG-version 1 (SEQ ID NO: 68), 3×FLAG-version 2 (SEQ ID NO: 15), 2×HA (SEQ ID NO: 69), 10aa (SEQ ID NO: 16), H2B (SEQ ID NO: 17), WPRE3 (SEQ ID NO: 18), WPRE (SEQ ID NO: 19), BGHpA (SEQ ID NO: 20), hGHpA (SEQ ID NO: 21), SV40 pA (SEQ ID NO: 70), ShortPolyA (SEQ ID NO: 71), IRES2 (SEQ ID NO: 72), P2A (SEQ ID NO: 22), T2A (SEQ ID NO: 23), E2A (SEQ ID NO: 24), F2A (SEQ ID NO: 25), Exemplary Plasmid Backbone 1—Left ITR (SEQ ID NO: 26), Exemplary Plasmid Backbone 1—Right ITR: (SEQ ID NO: 27), Exemplary Plasmid Backbone 2—Left ITR (SEQ ID NO: 28), Exemplary Plasmid Backbone 2—Right ITR (SEQ ID NO: 29), PHP.eB capsid (SEQ ID NO: 30), AAV9 VP1 capsid protein (SEQ ID NO: 31), tet-Transactivator version 2 (tTA2) (SEQ ID NO: 32), GTPase HRas [Homo sapiens](SEQ ID NO: 33), CN3252 (SEQ ID NO: 73), CN3254 (SEQ ID NO: 74), CN3683 (SEQ ID NO: 75), CN3684 (SEQ ID NO: 76), CN3251 (SEQ ID NO: 77), CN3253 (SEQ ID NO: 78), CN3677 (SEQ ID NO: 79), CN3678 (SEQ ID NO: 80), CN4541 (SEQ ID NO: 81), CN4542 (SEQ ID NO: 82), CN4217 (SEQ ID NO: 83), CN4218 (SEQ ID NO: 84), CN4642 (SEQ ID NO: 85), and CN4643 (SEQ ID NO: 86).

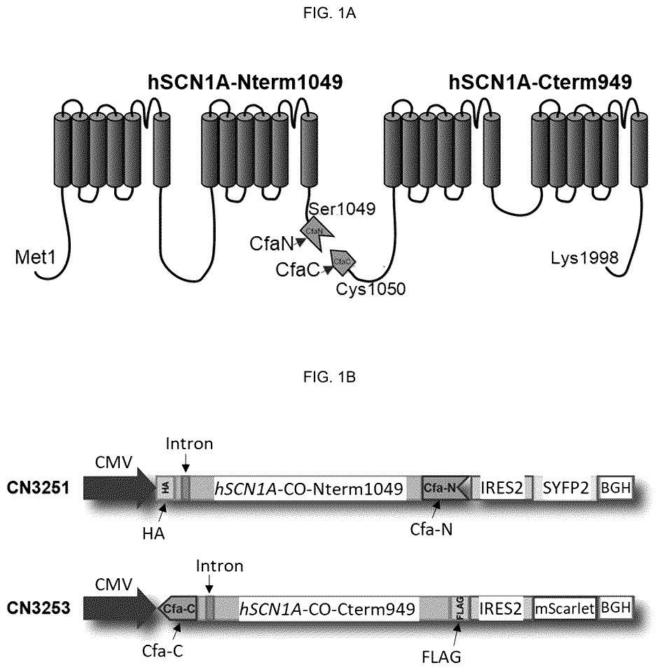

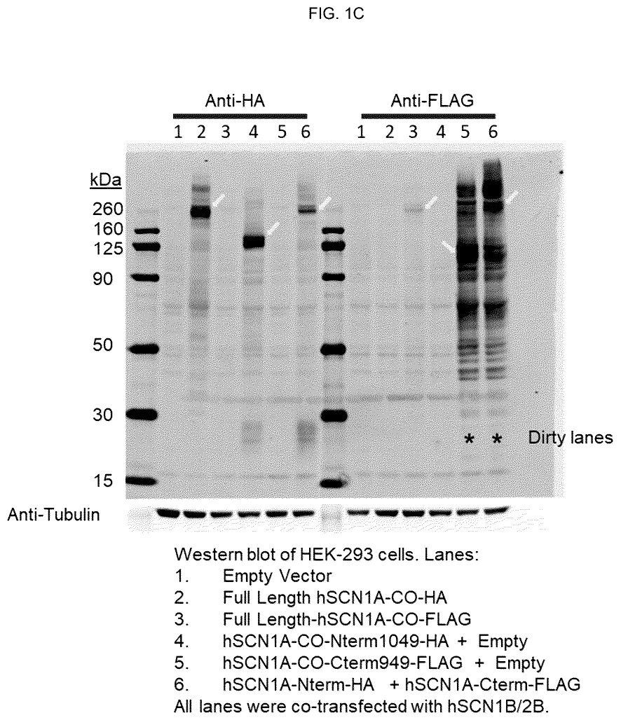

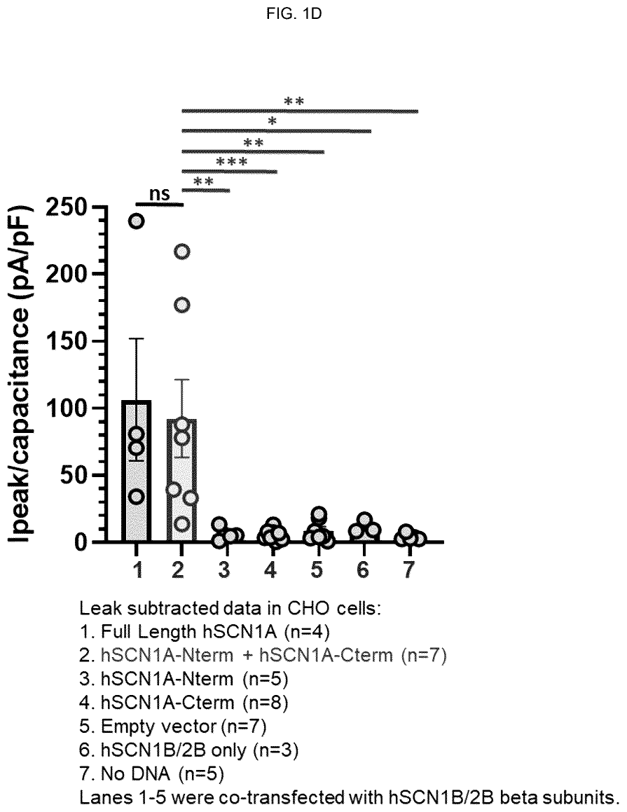

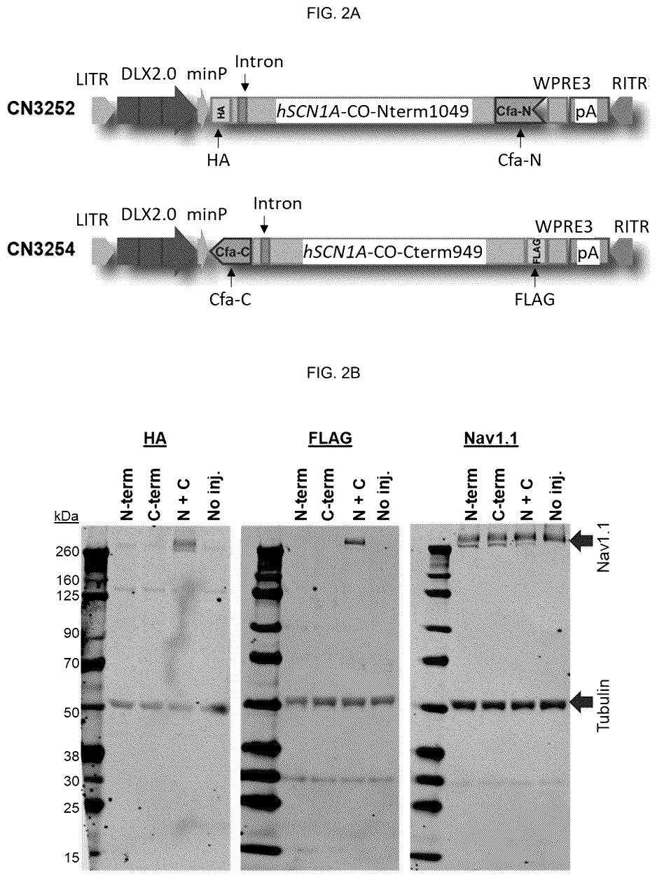

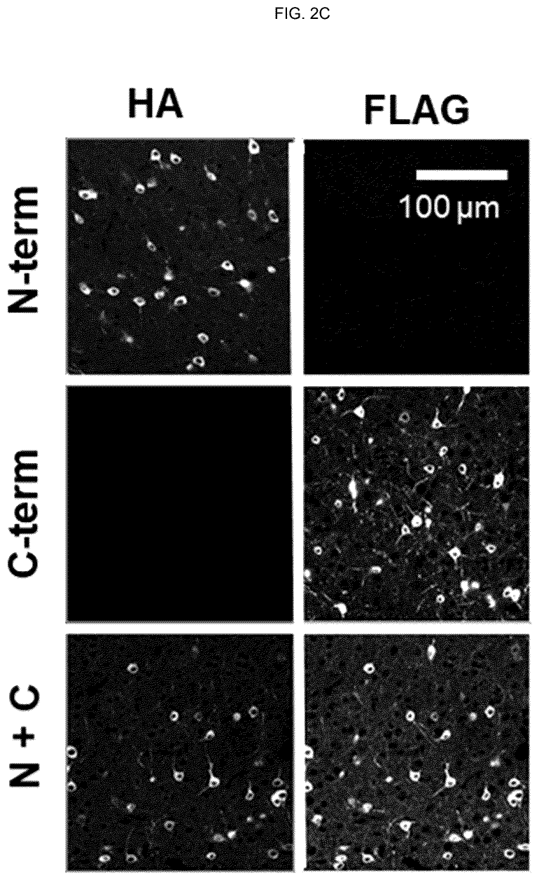

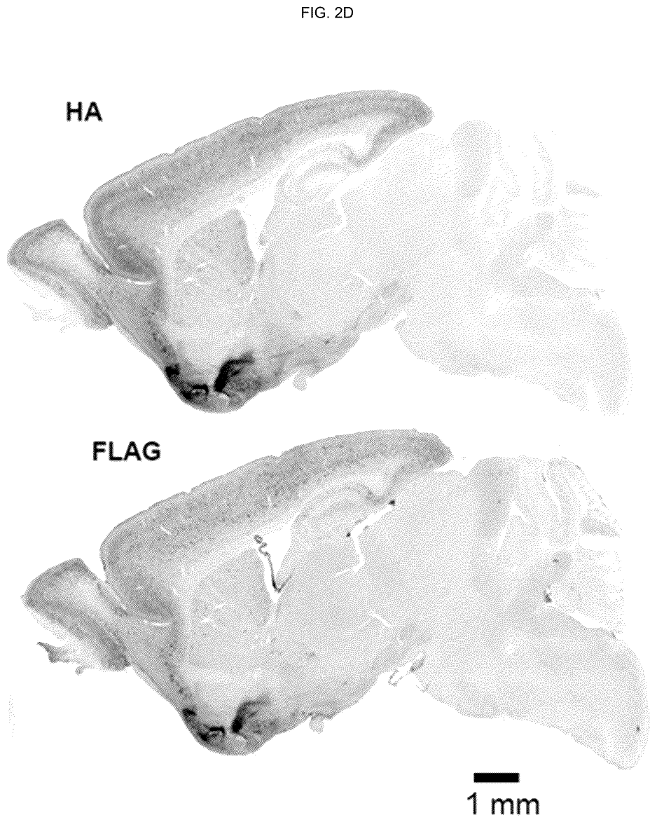

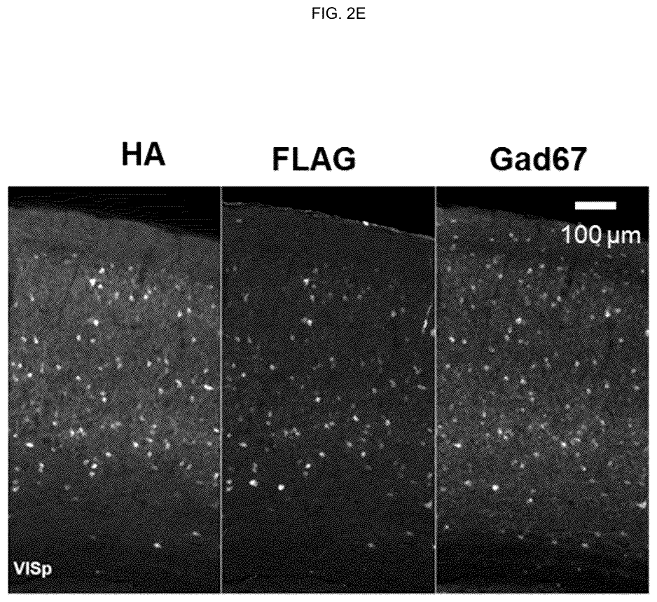

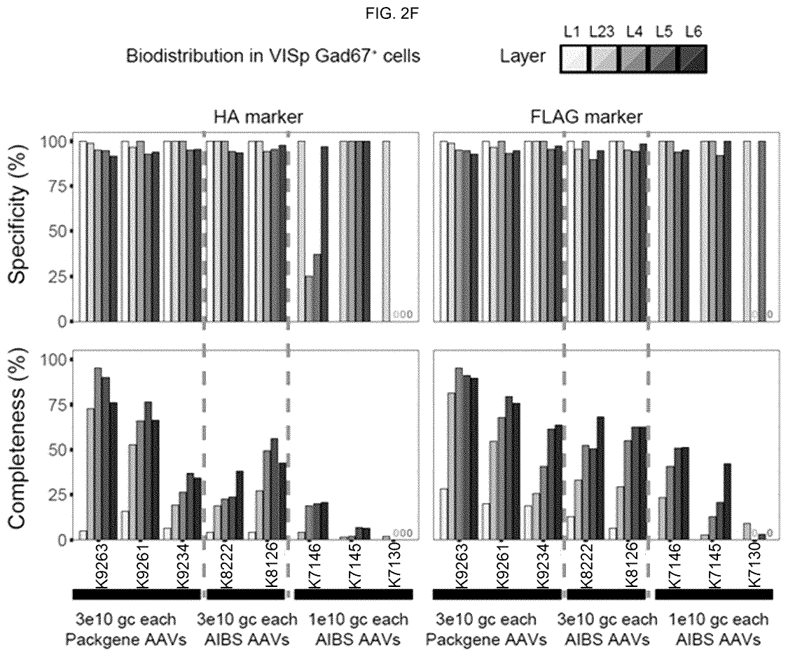

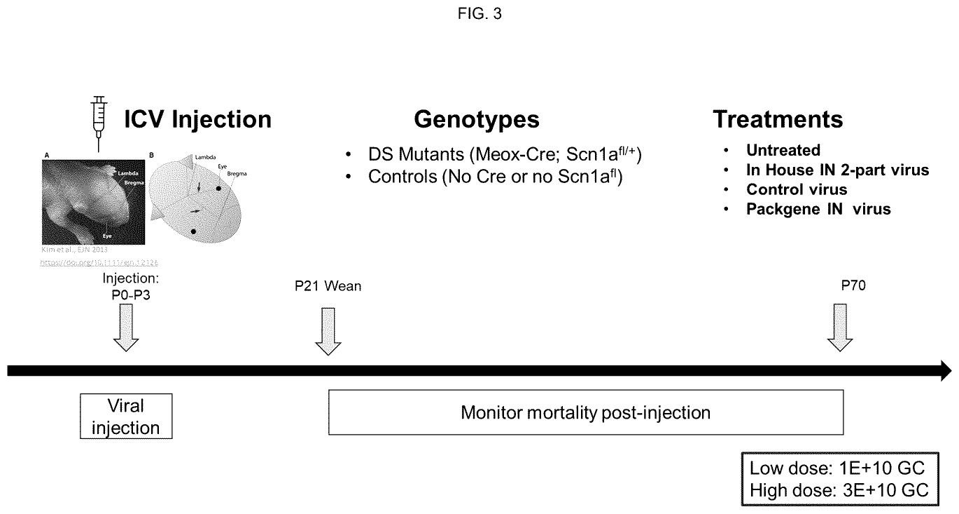

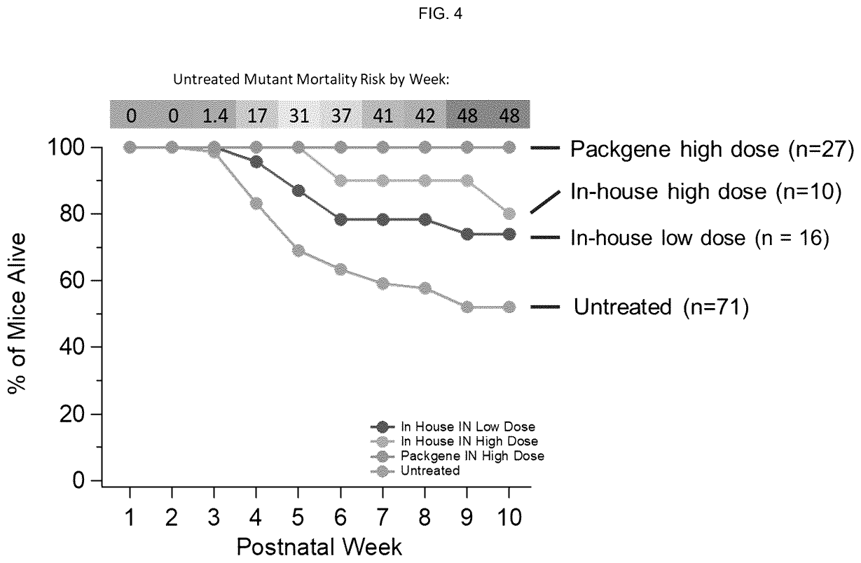

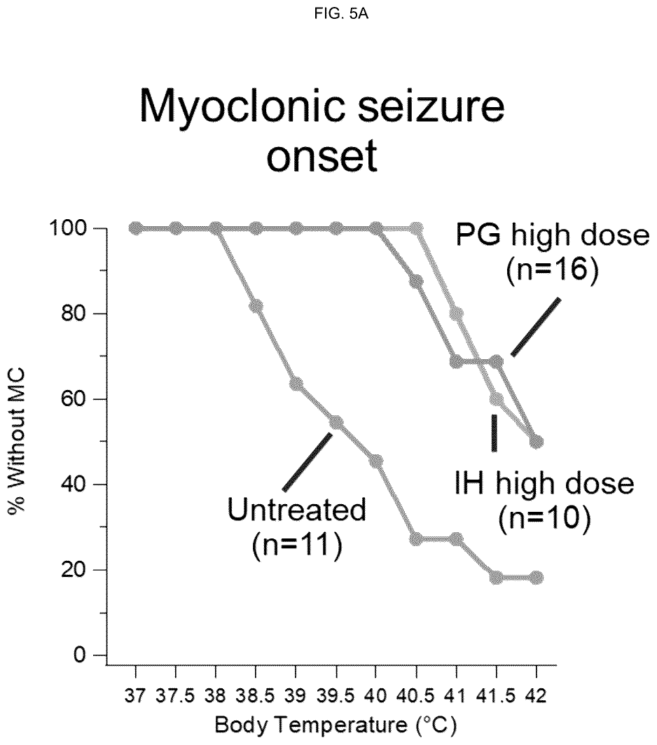

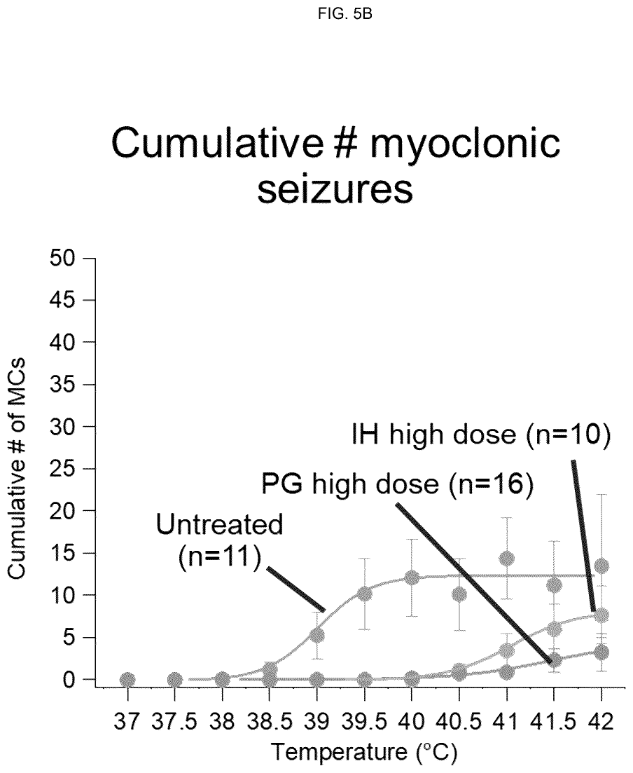

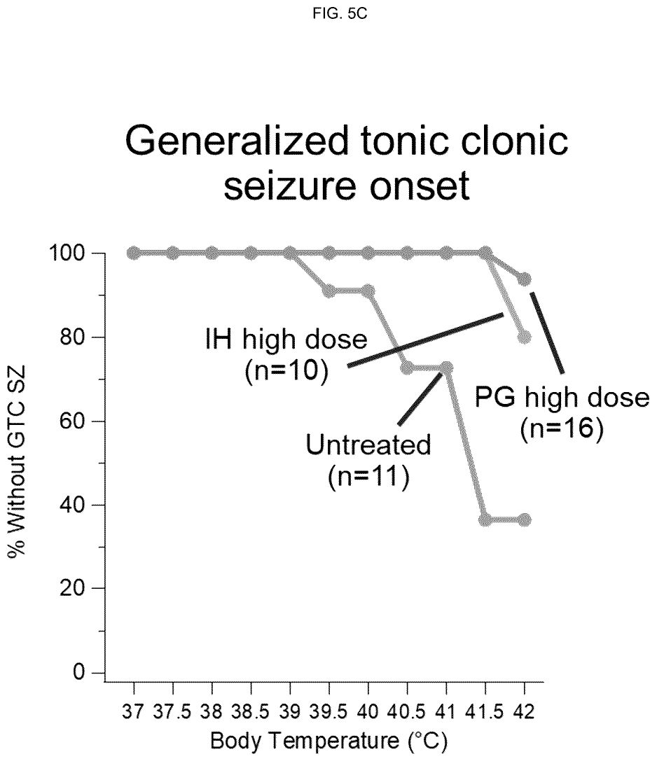

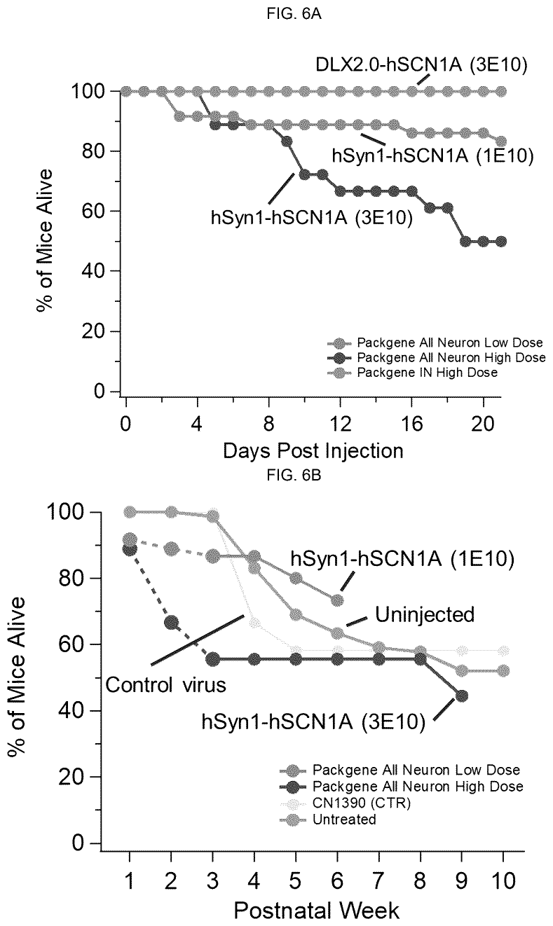

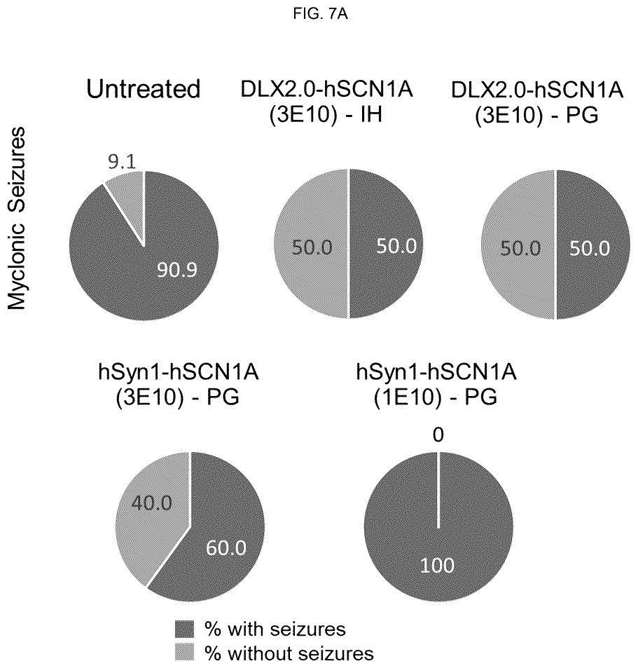

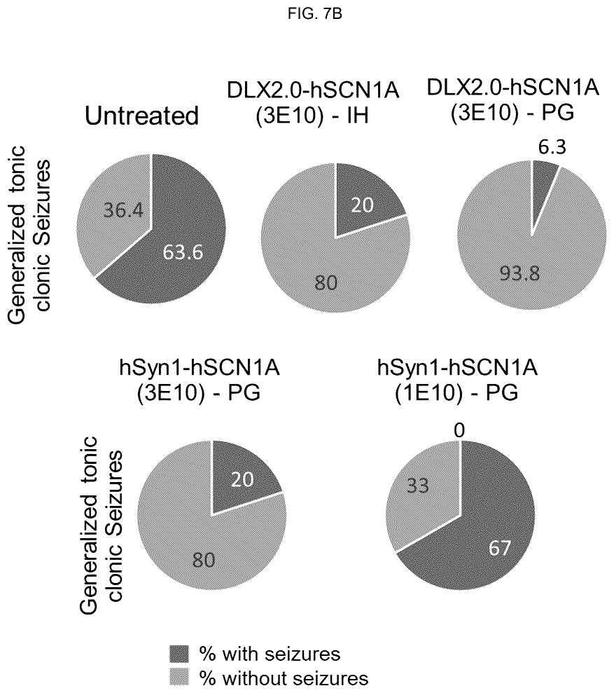

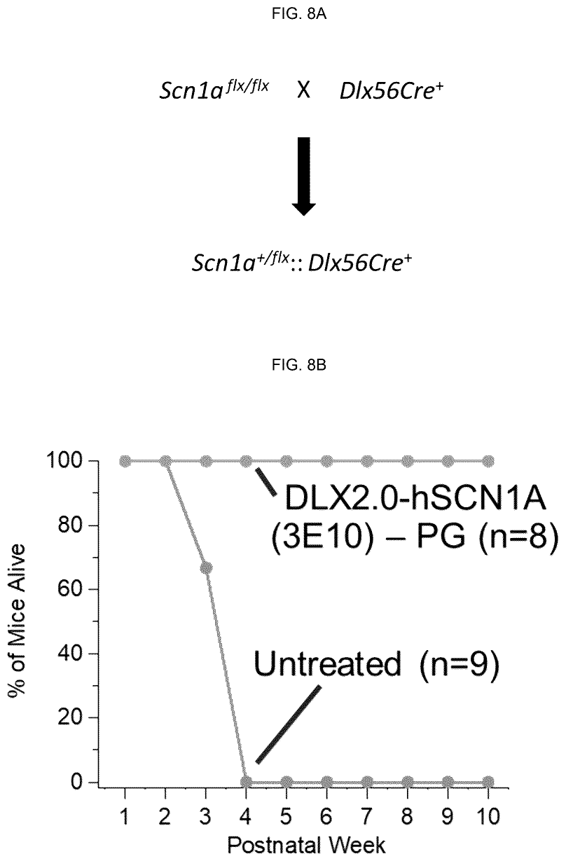

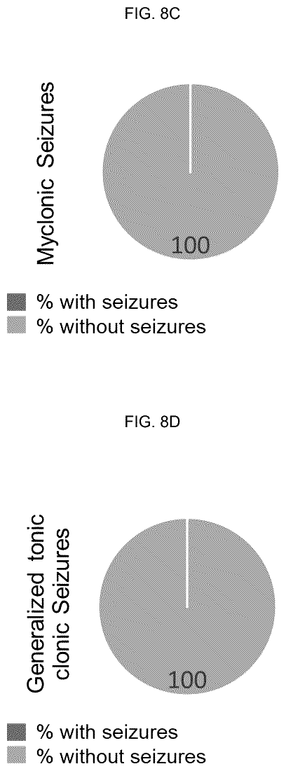

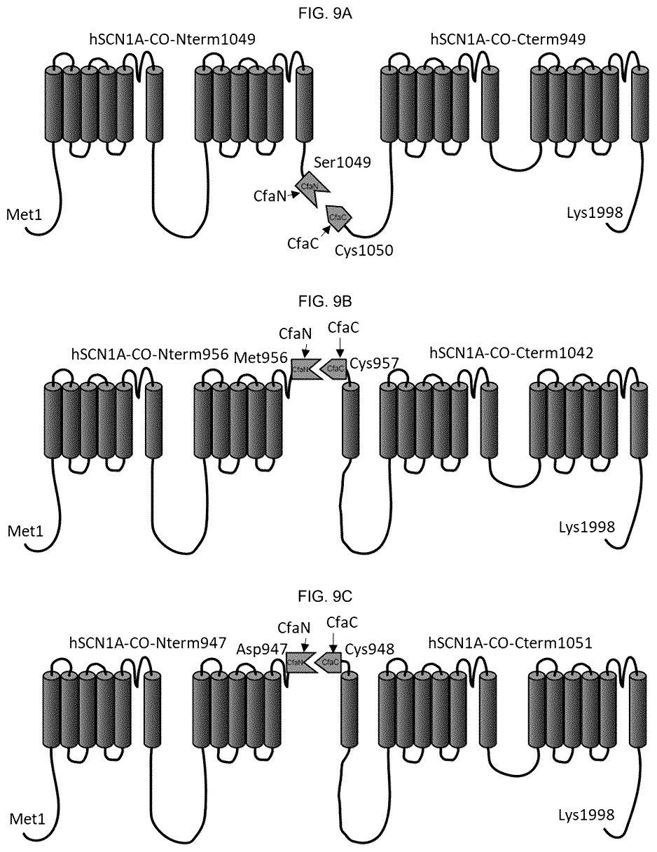

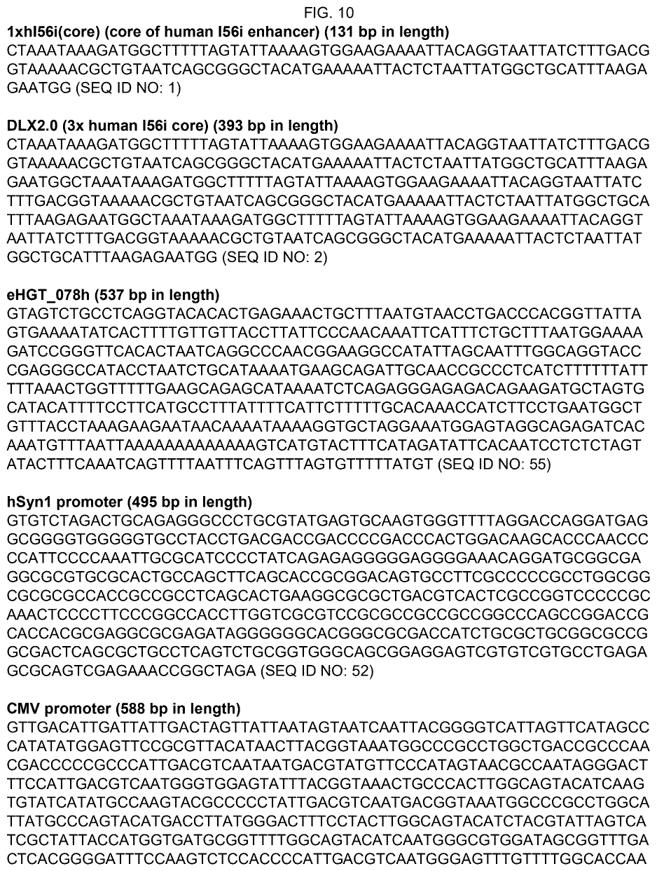

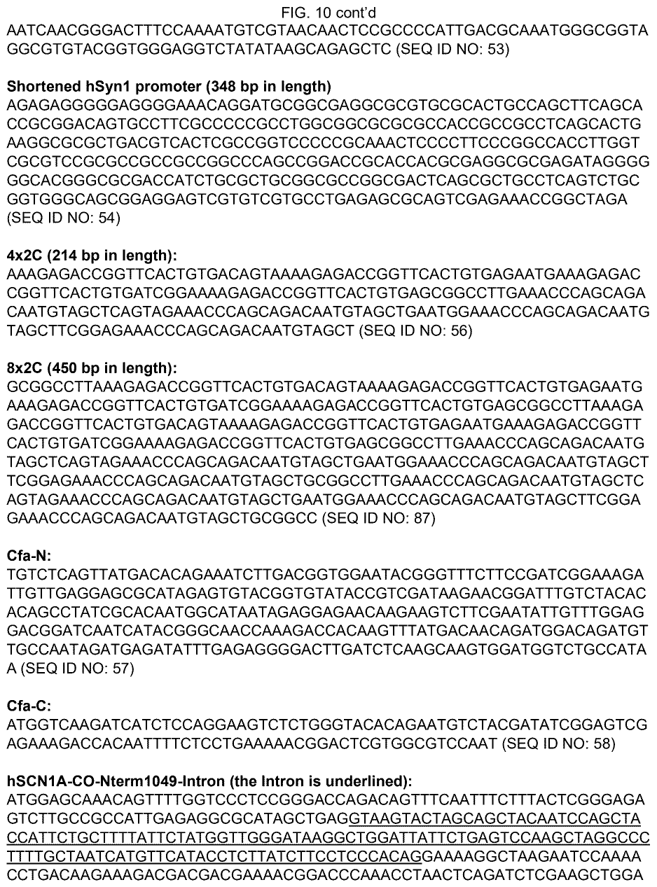

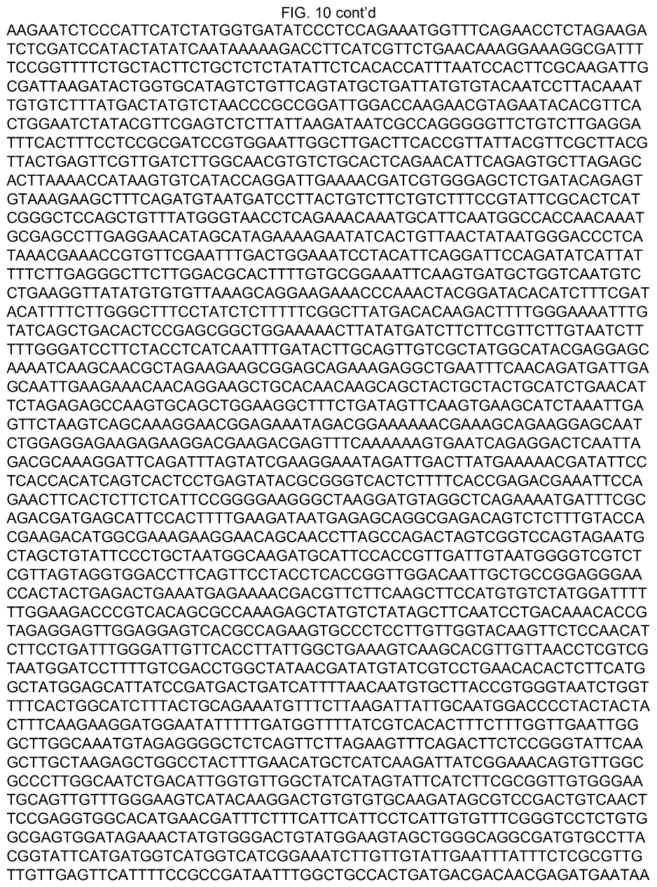

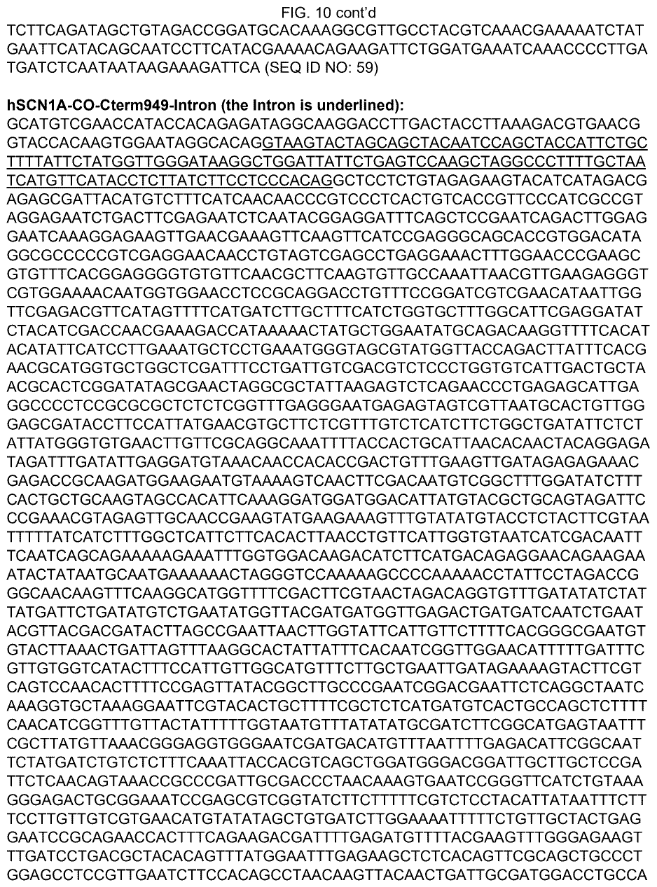

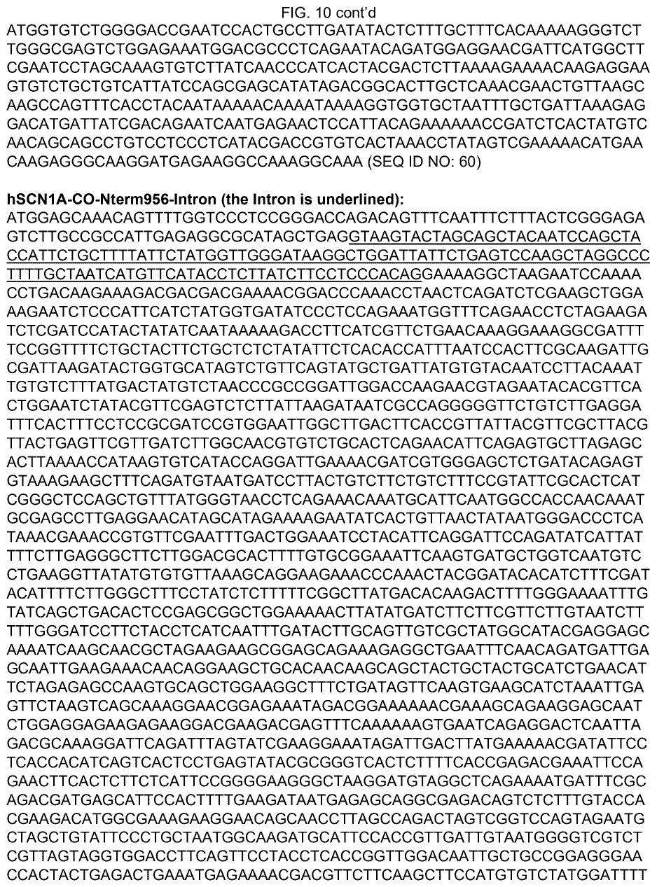

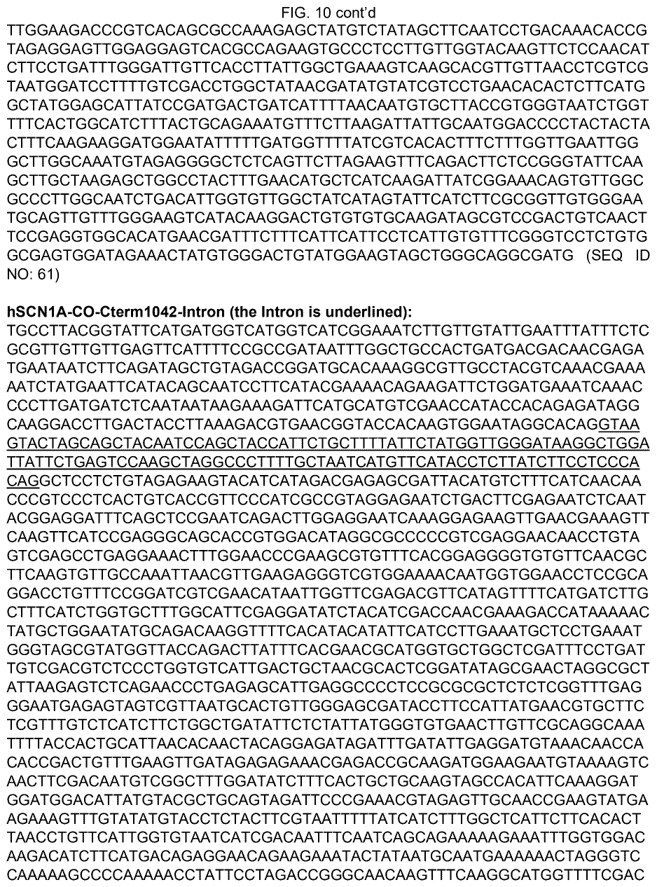

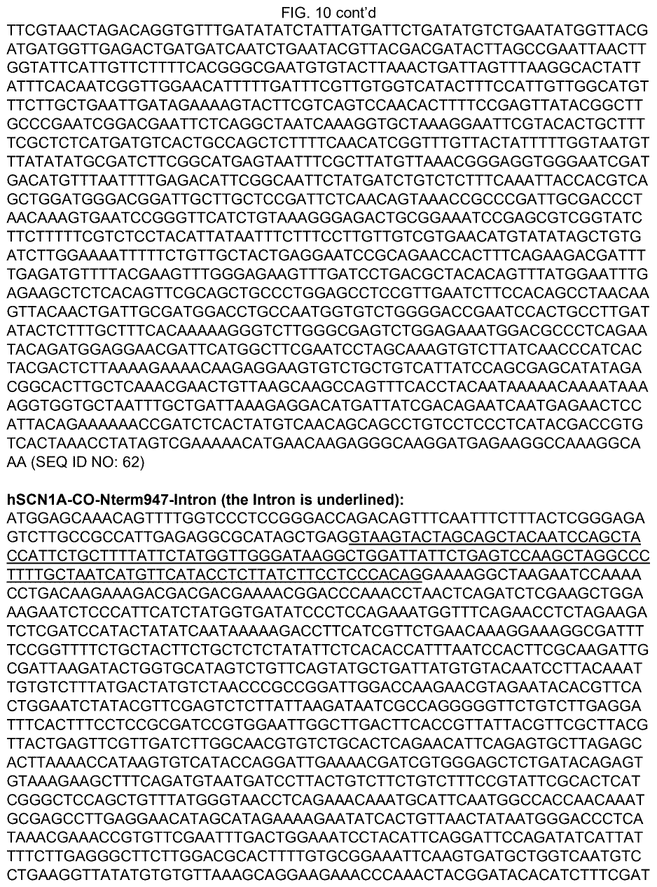

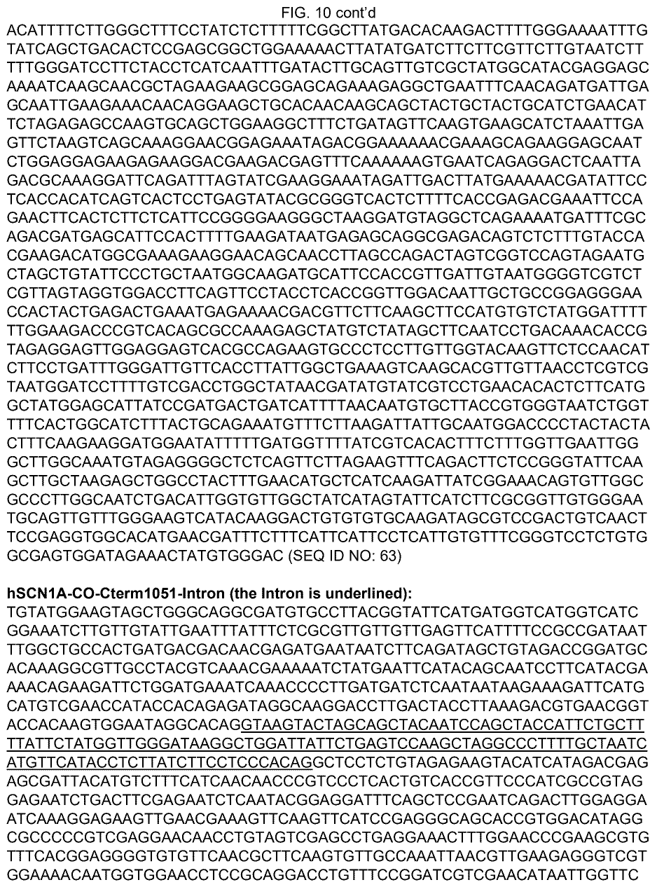

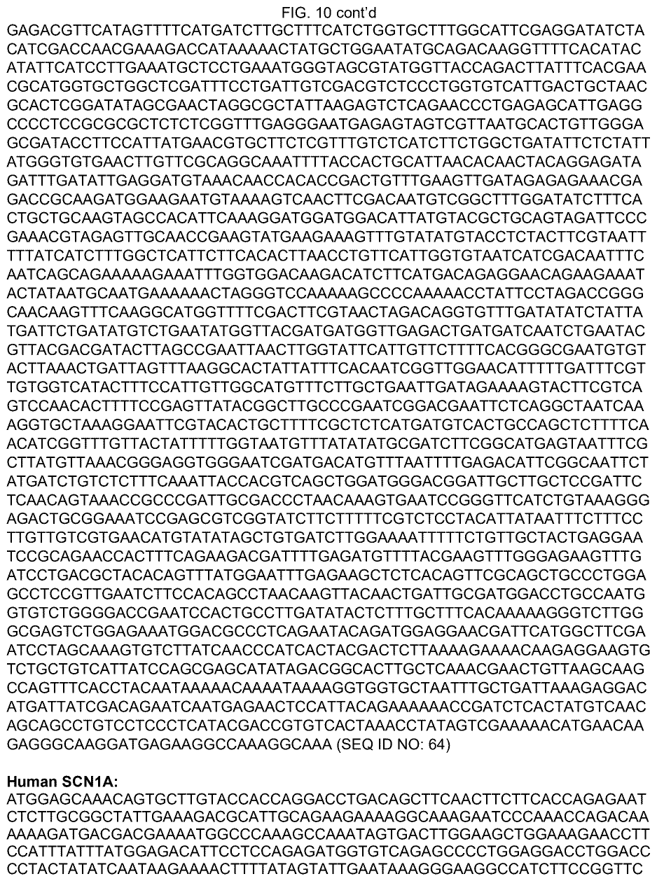

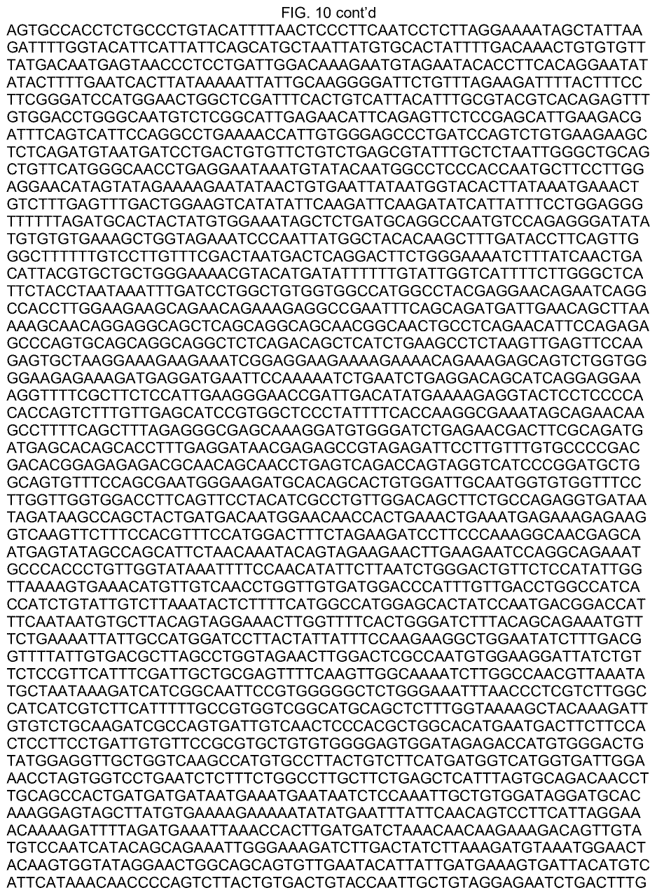

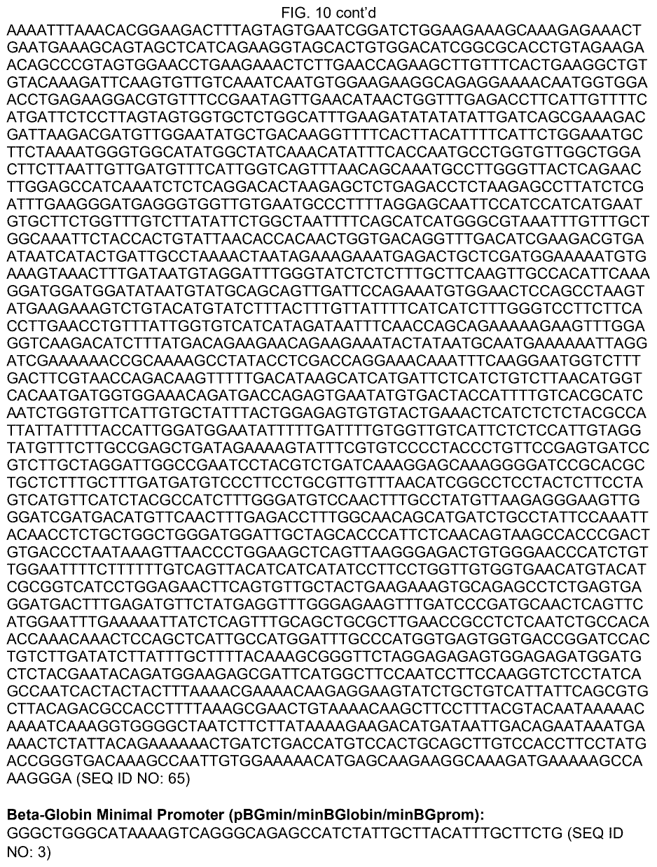

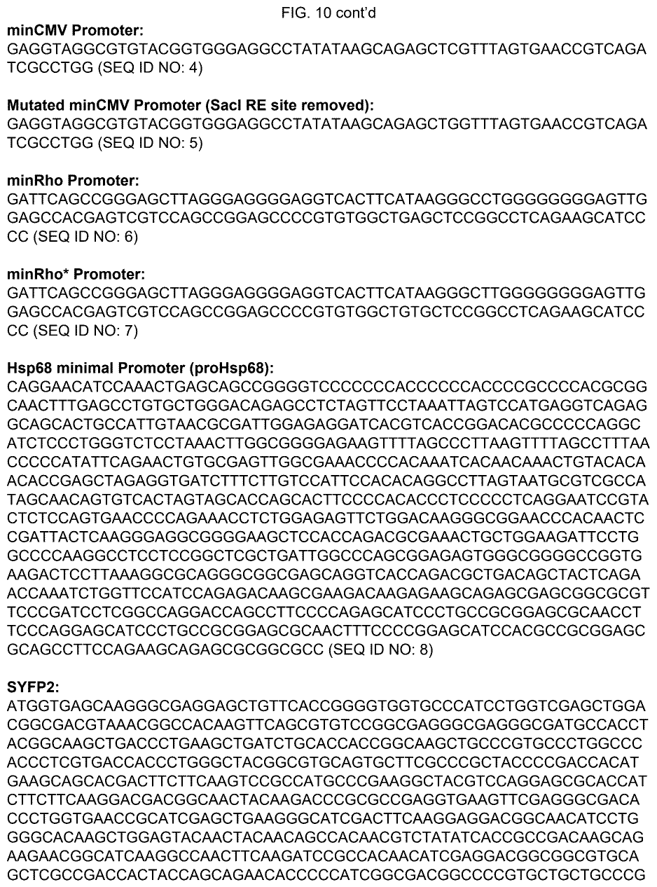

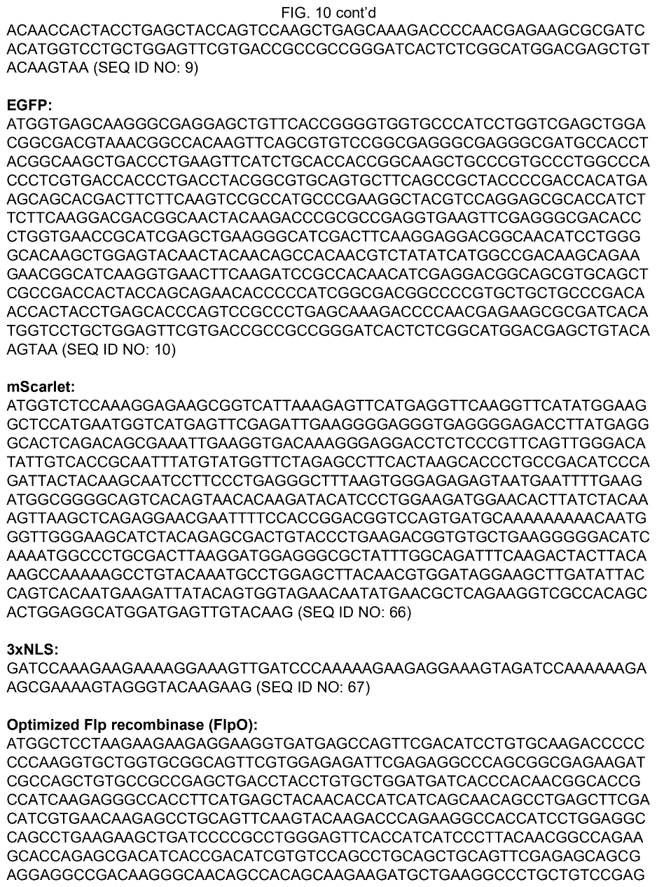

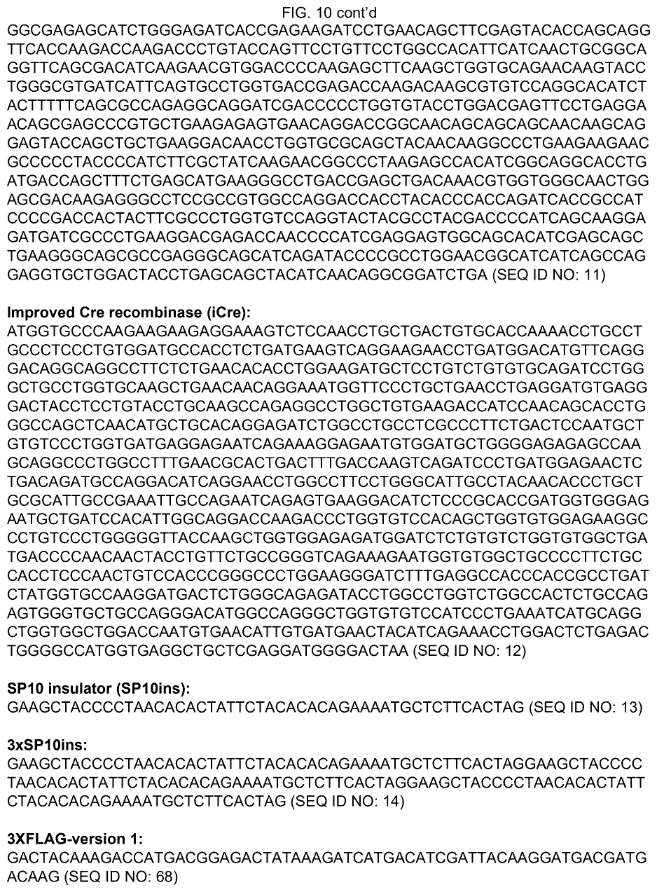

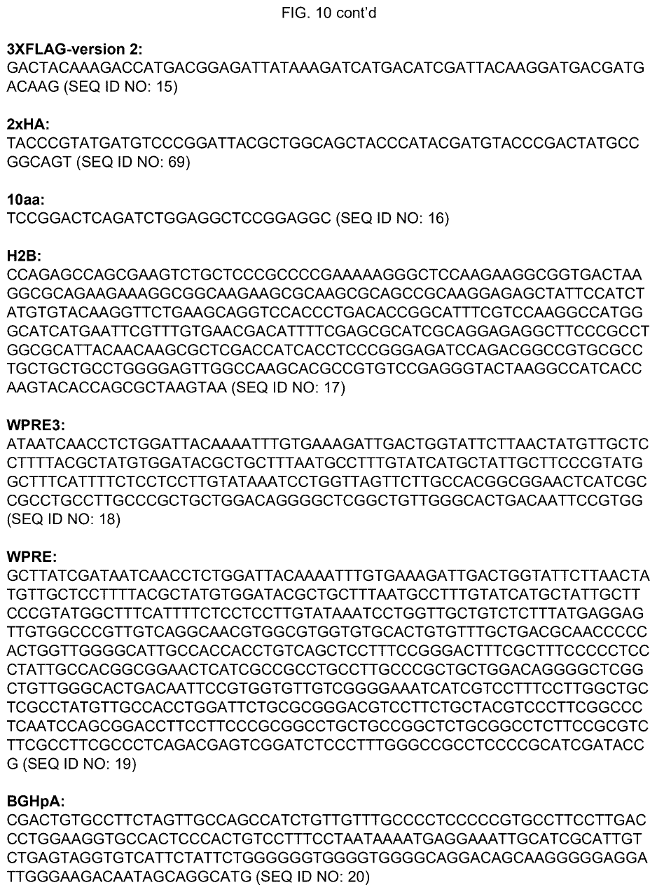

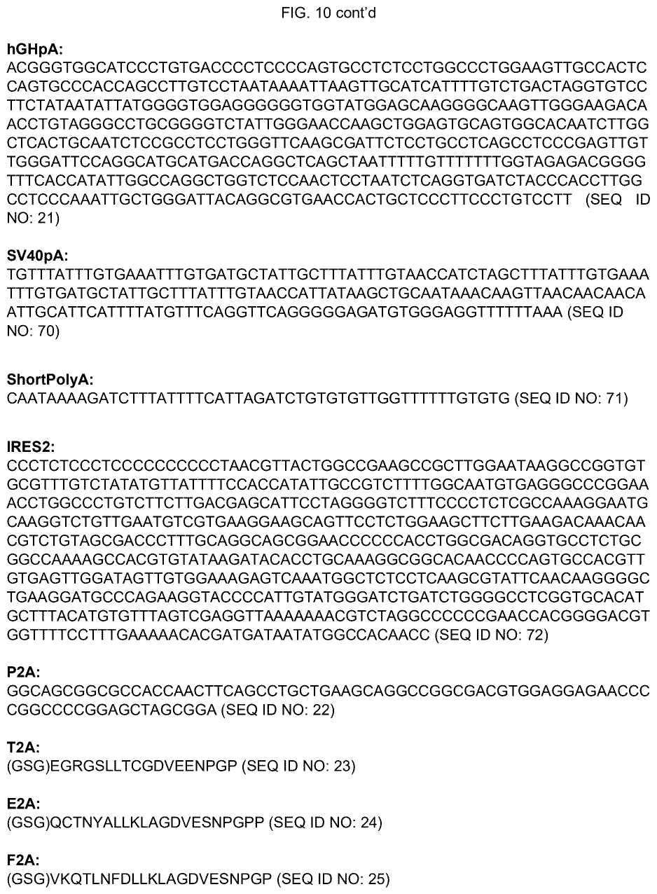

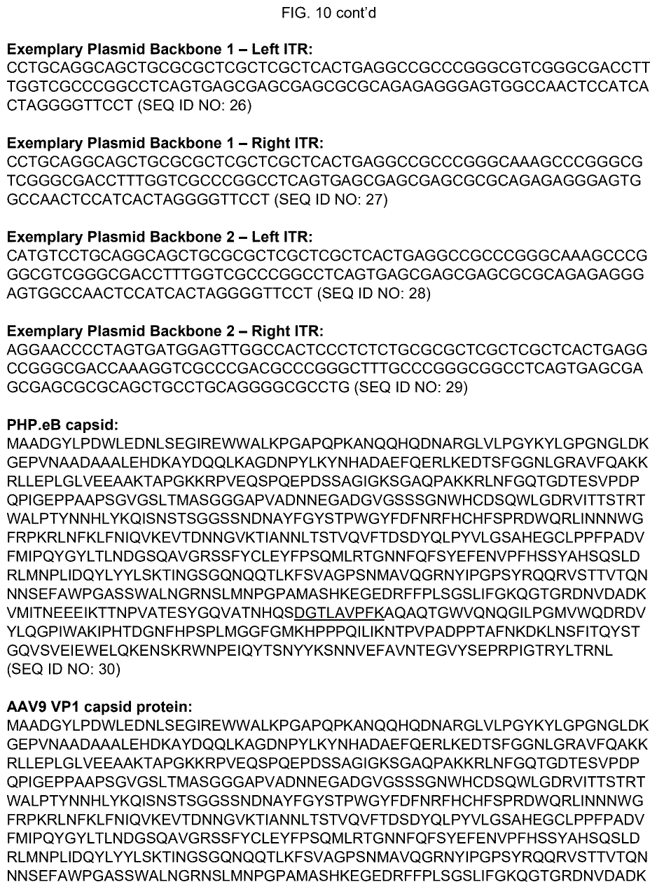

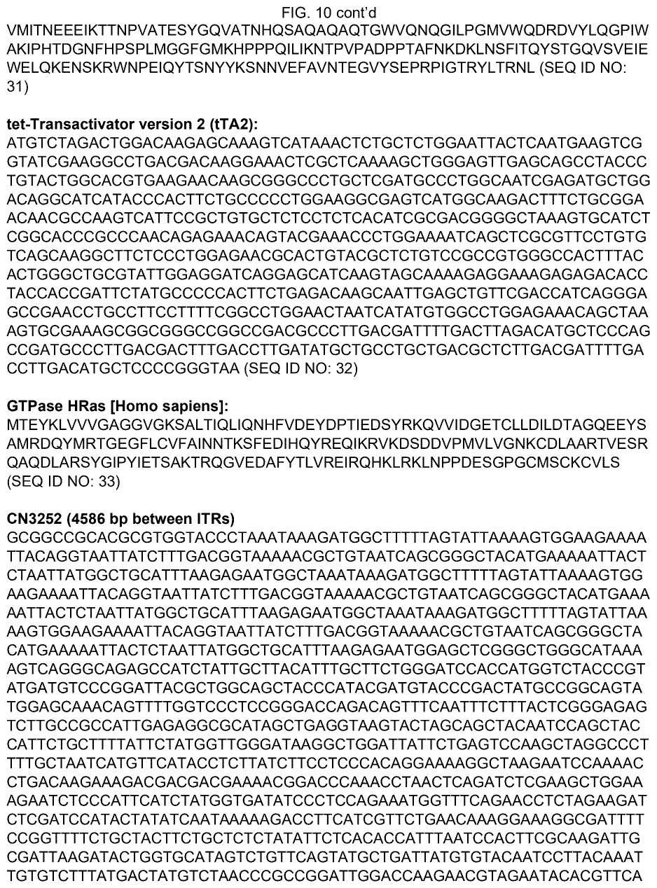

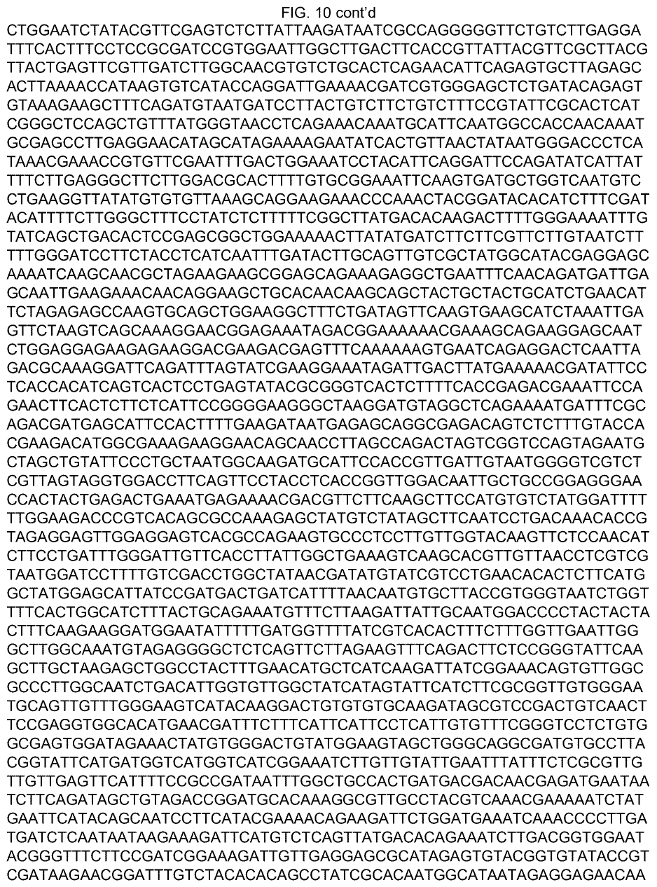

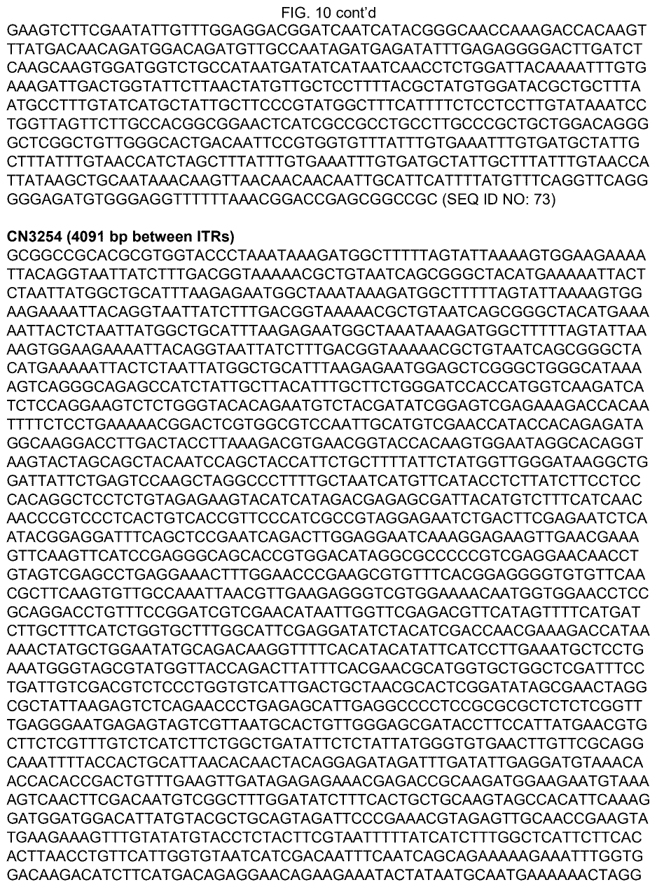

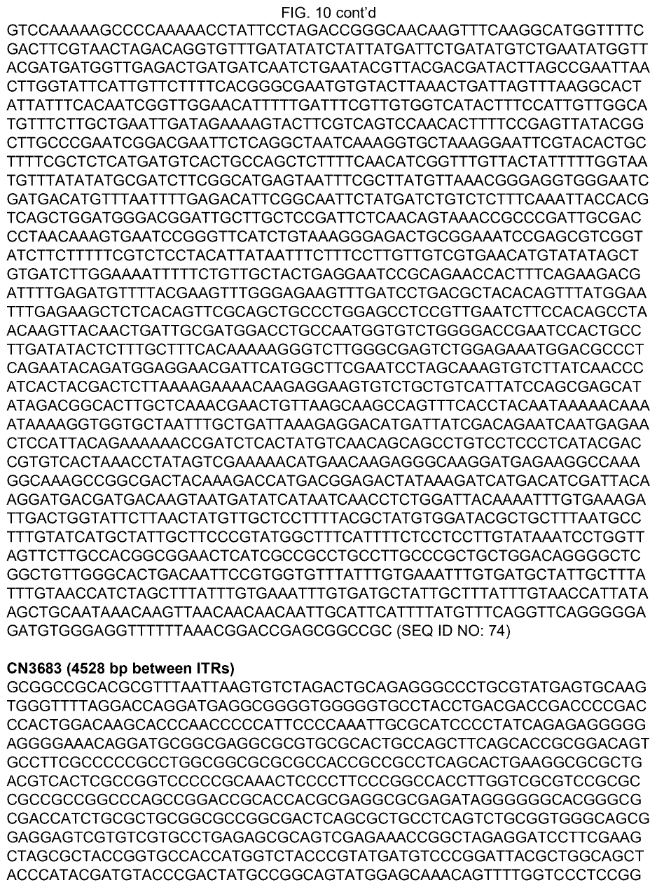

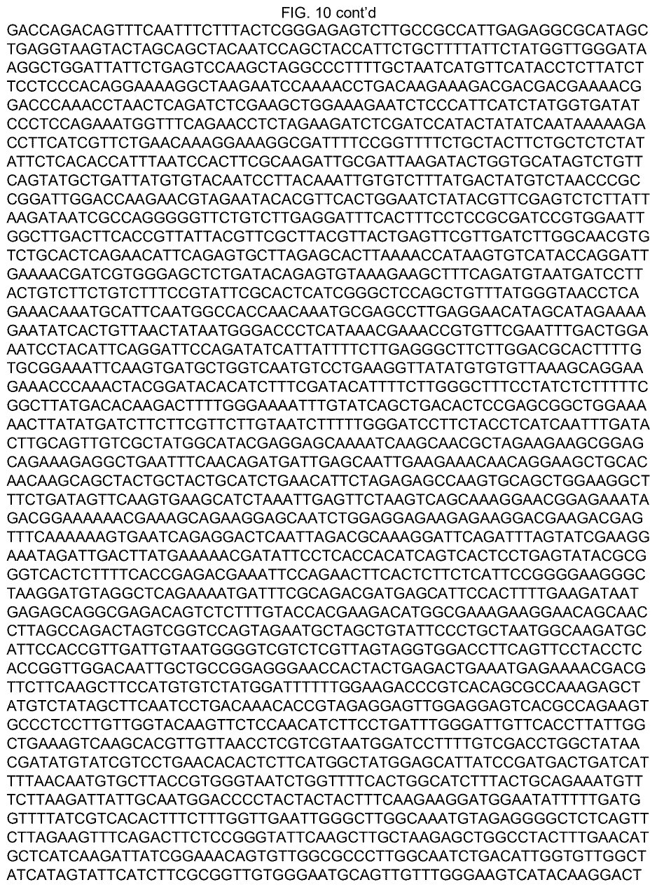

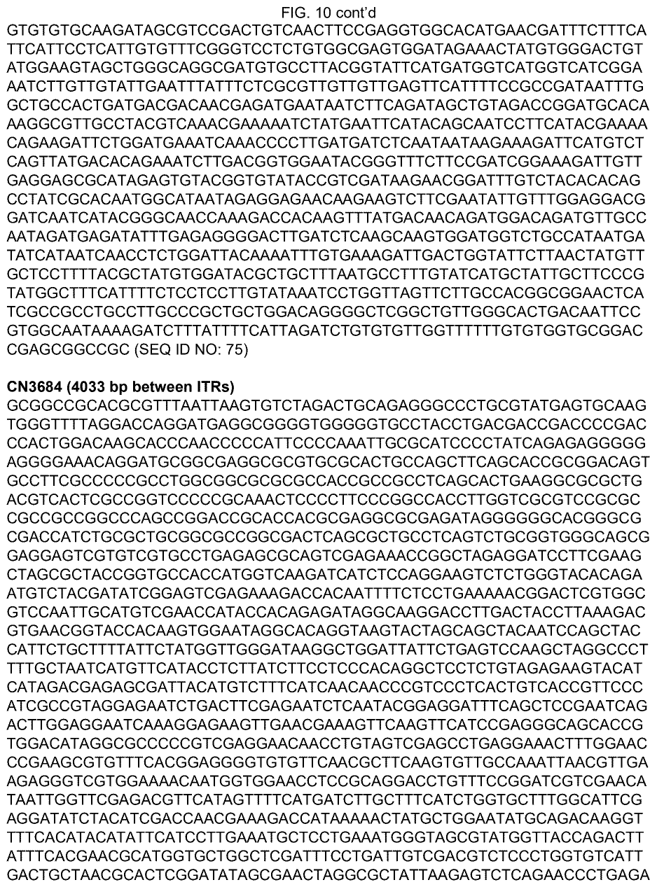

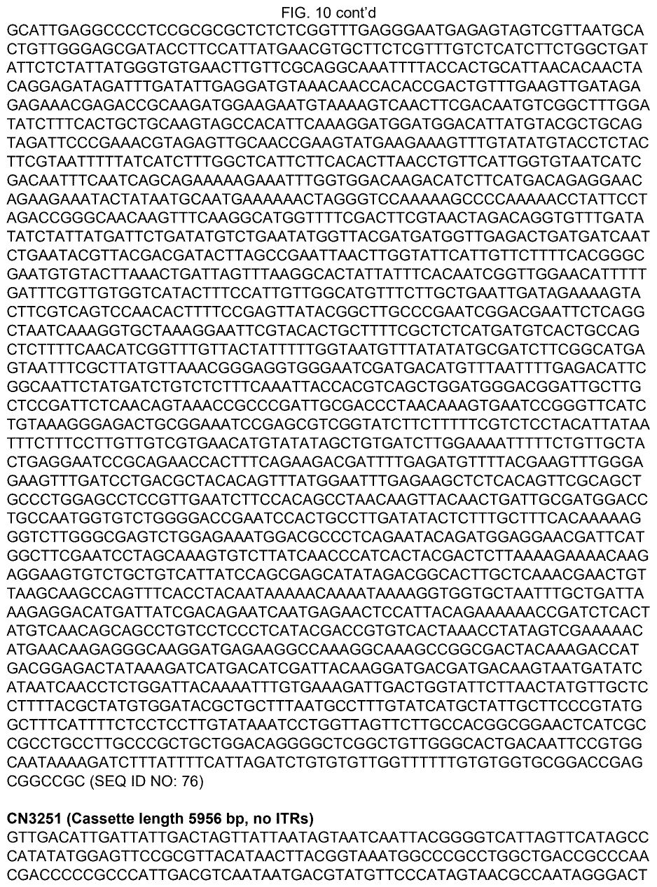

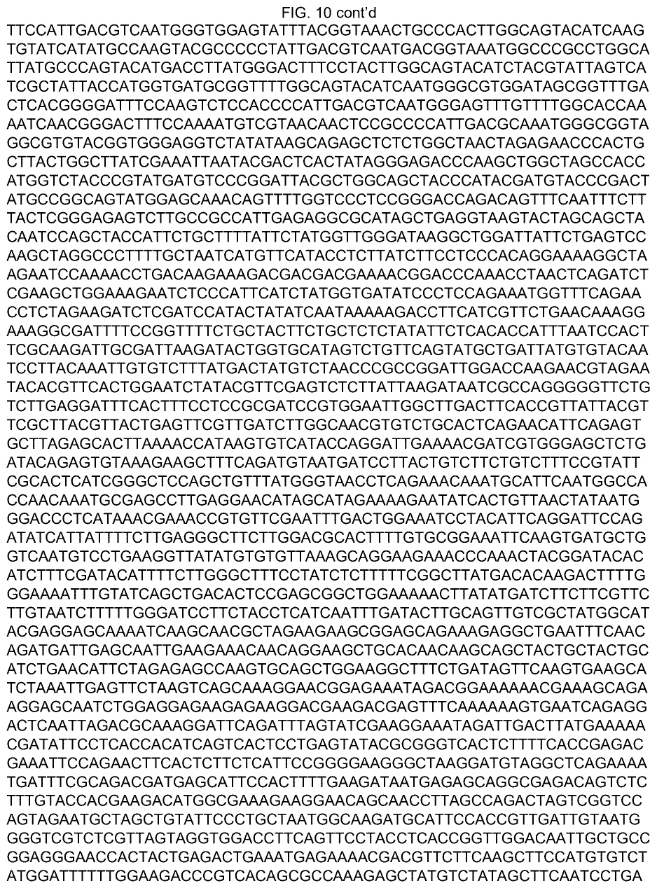

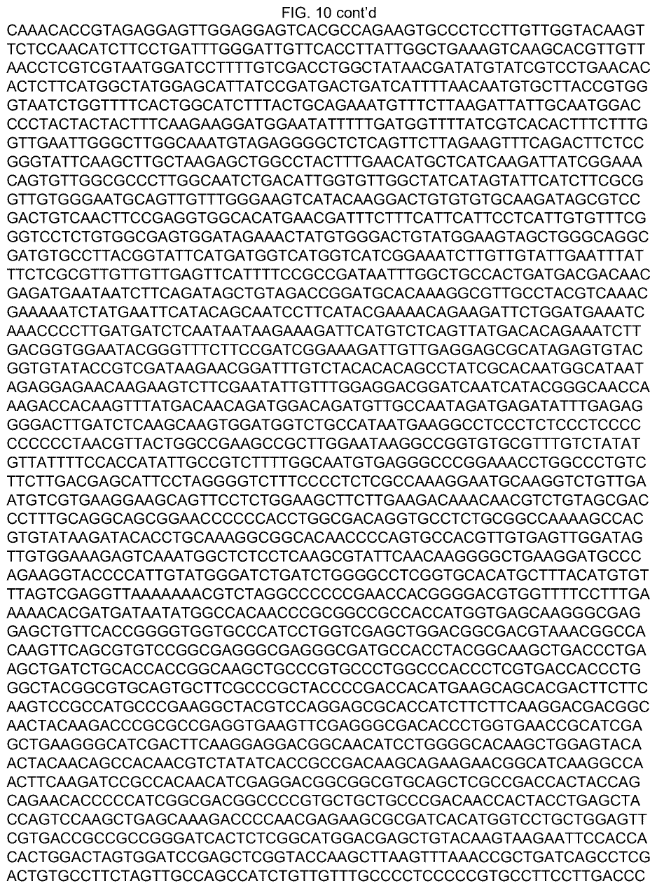

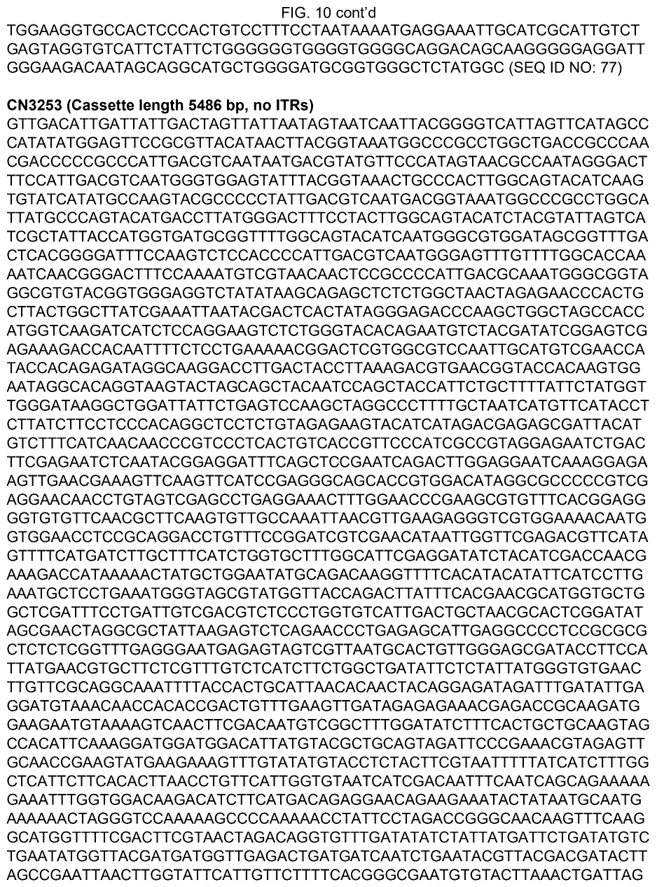

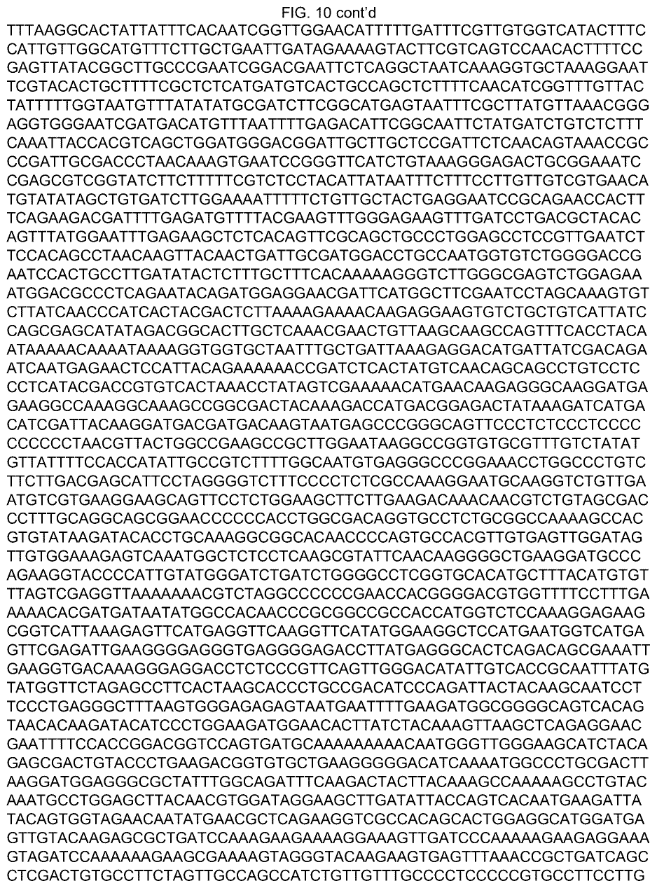

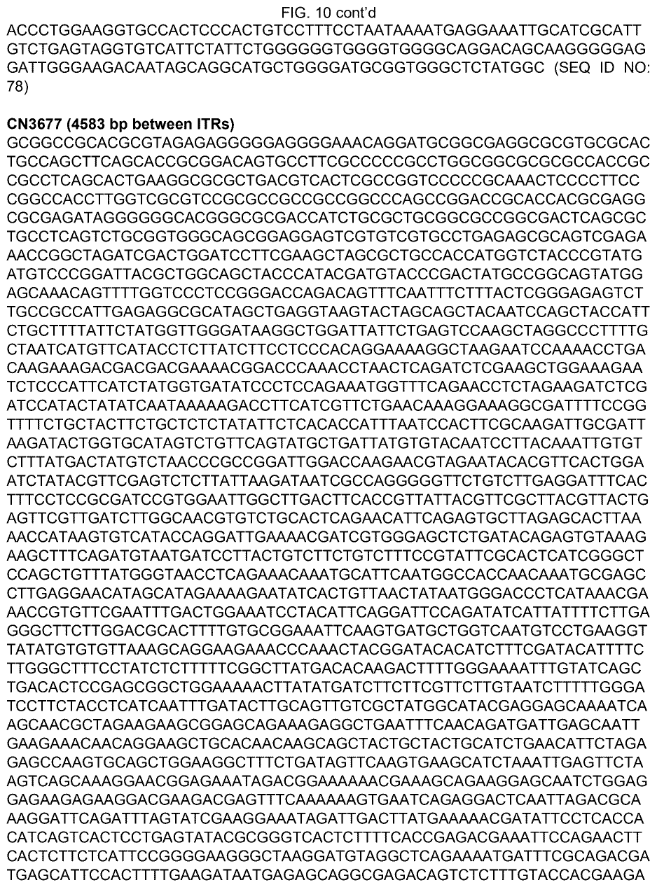

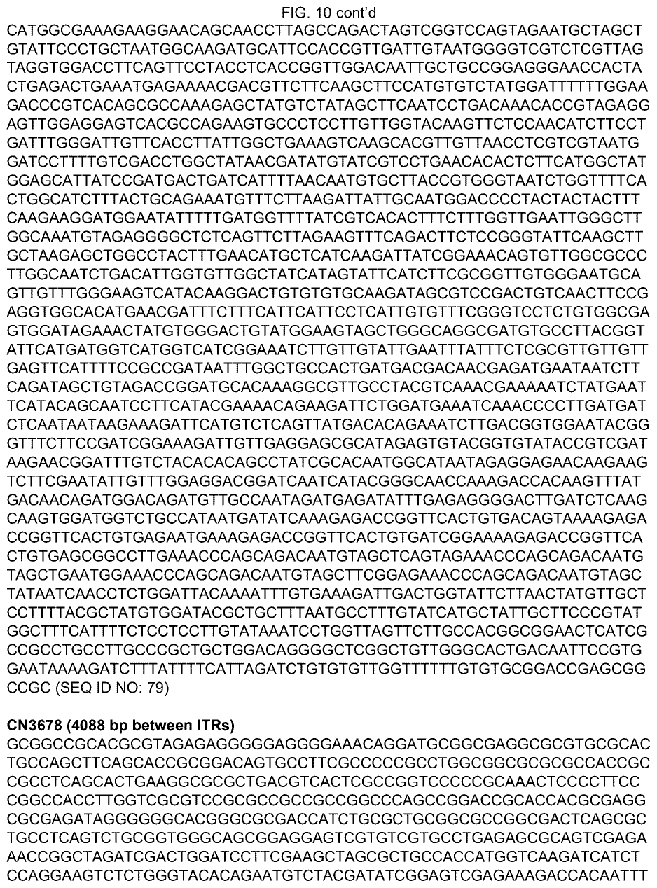

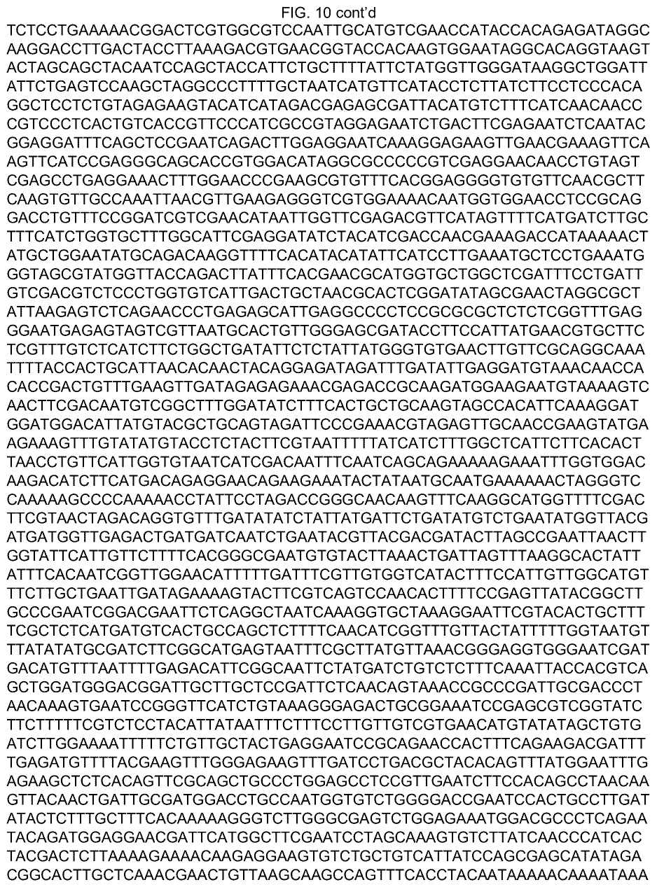

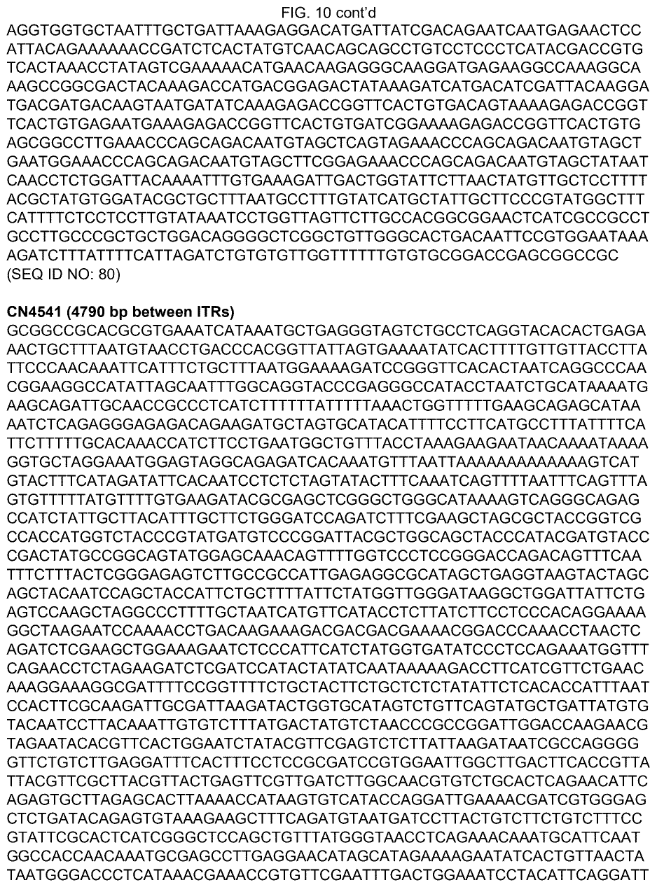

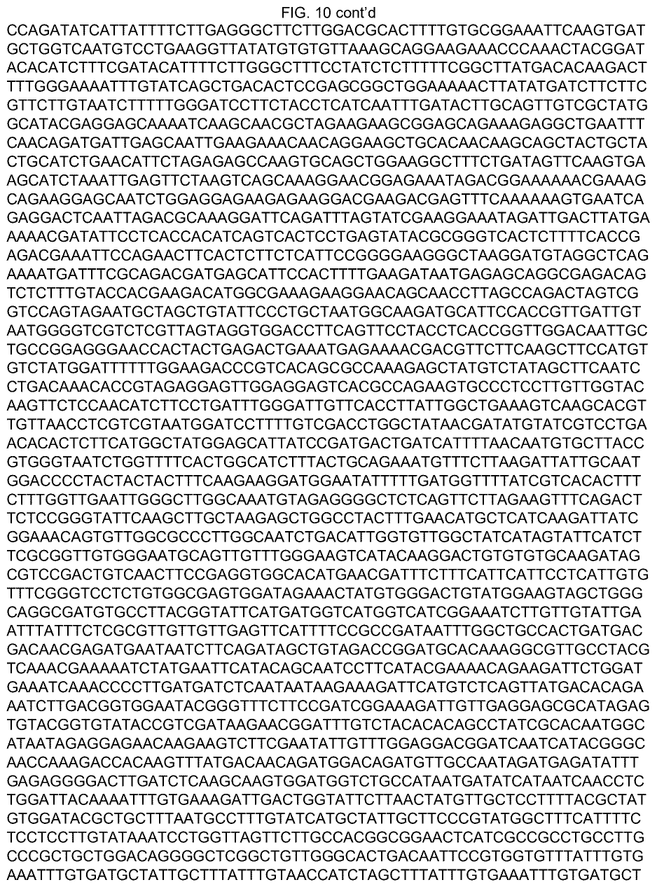

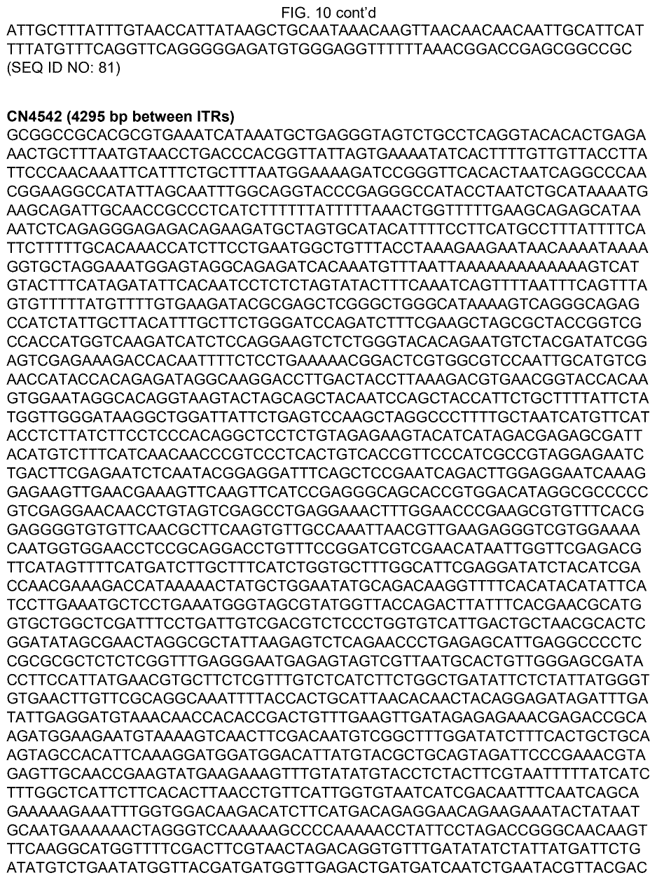

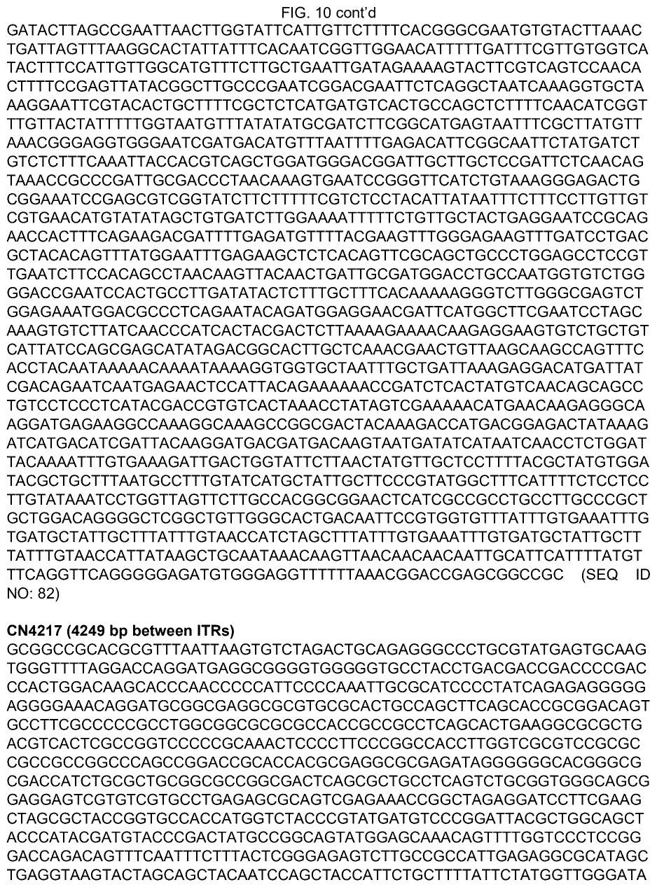

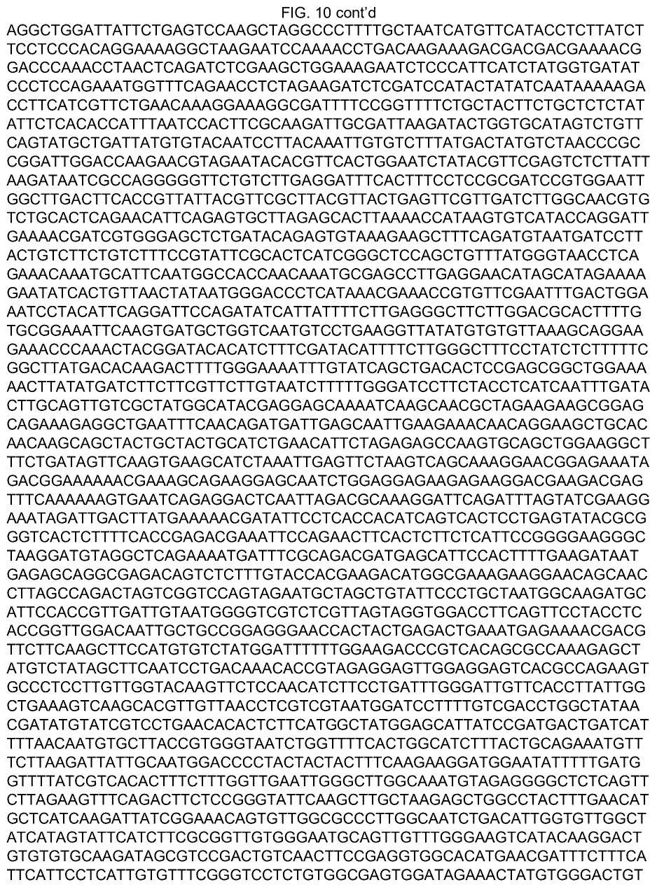

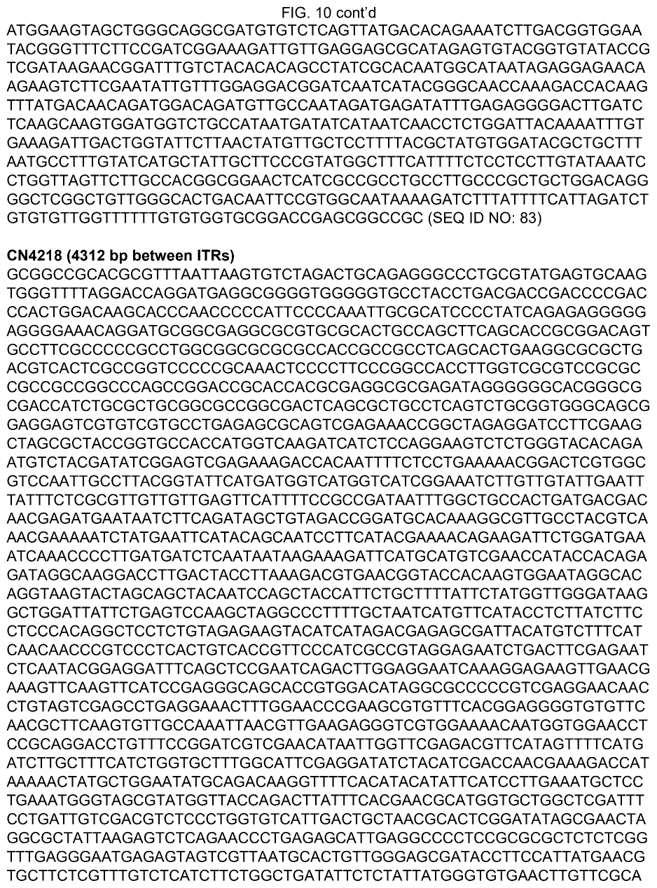

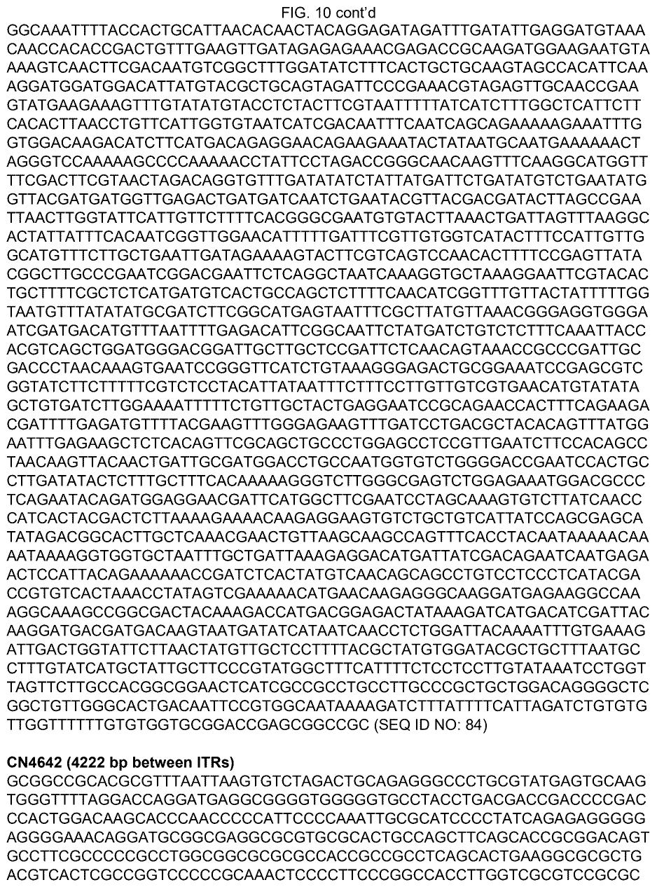

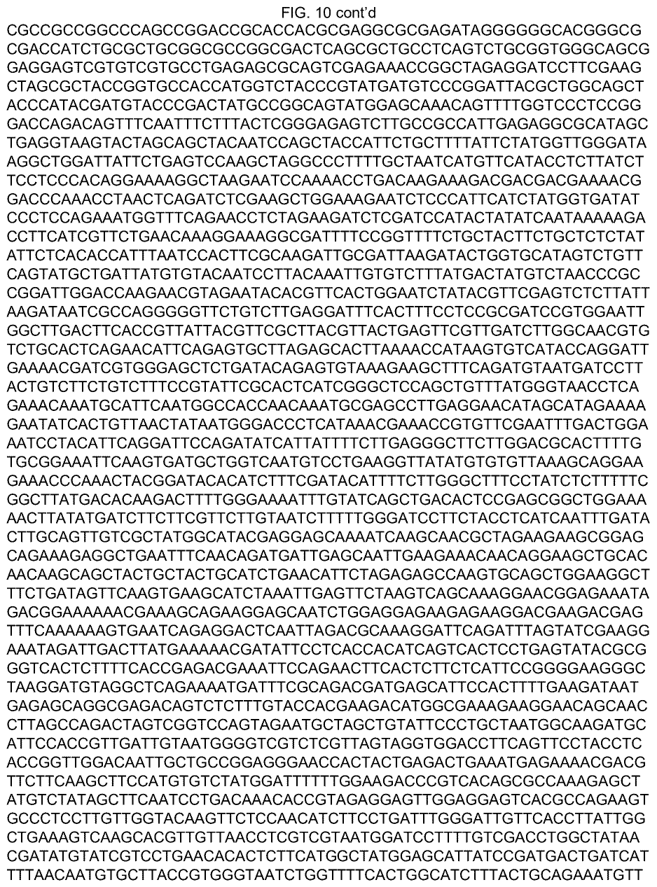

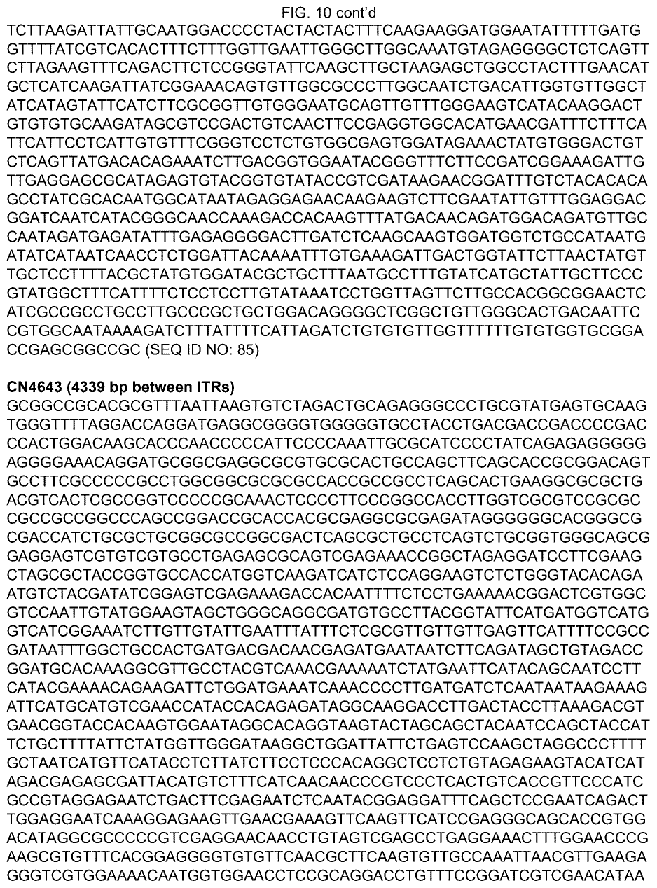

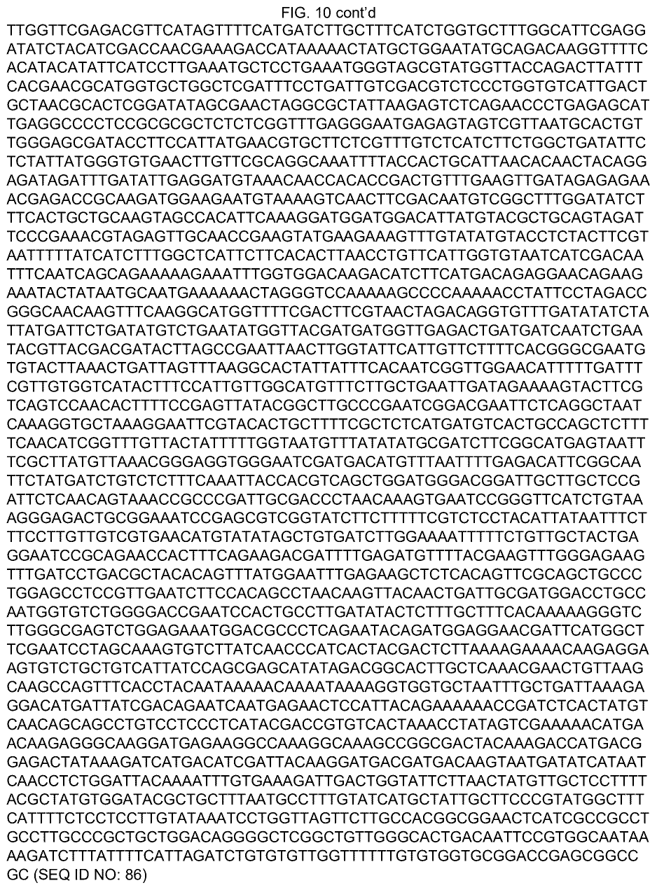

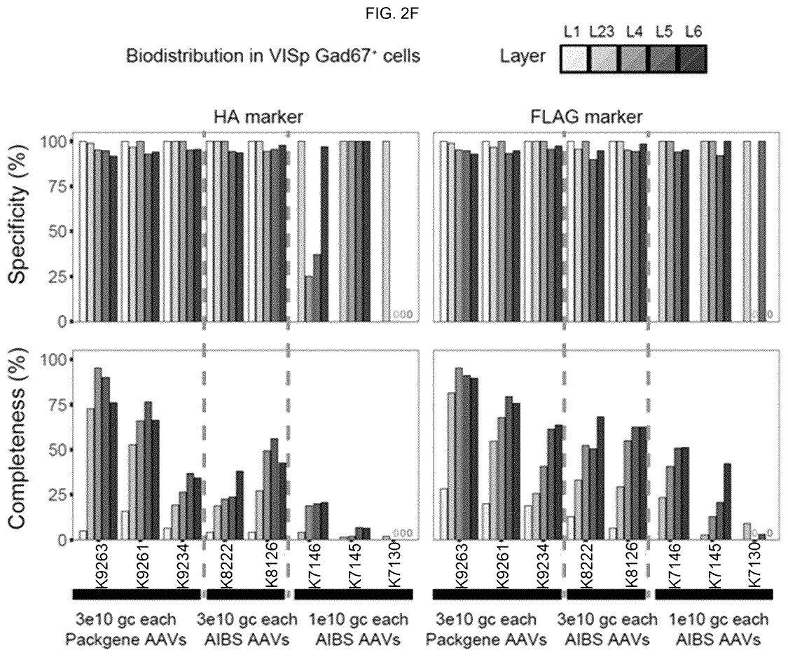

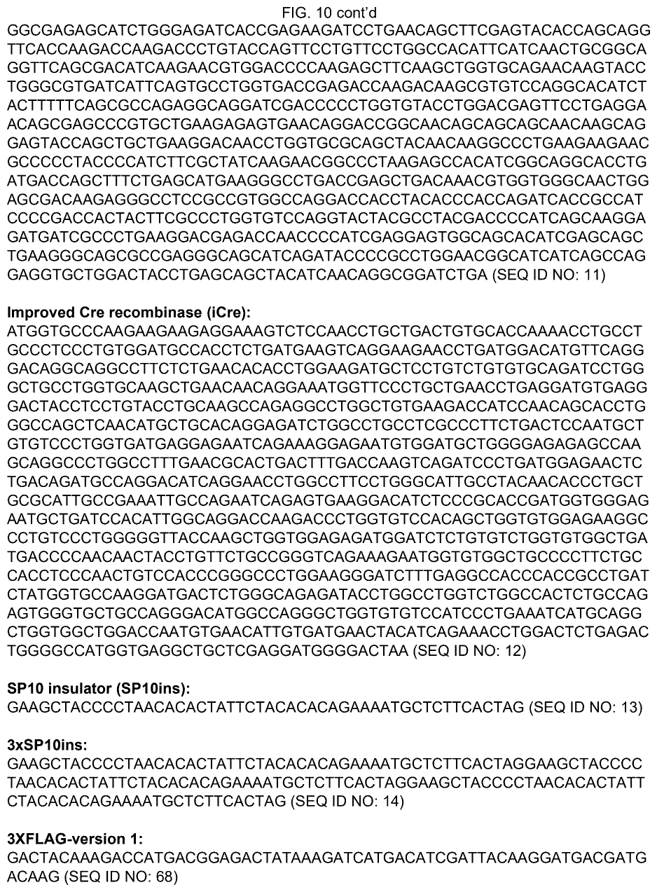

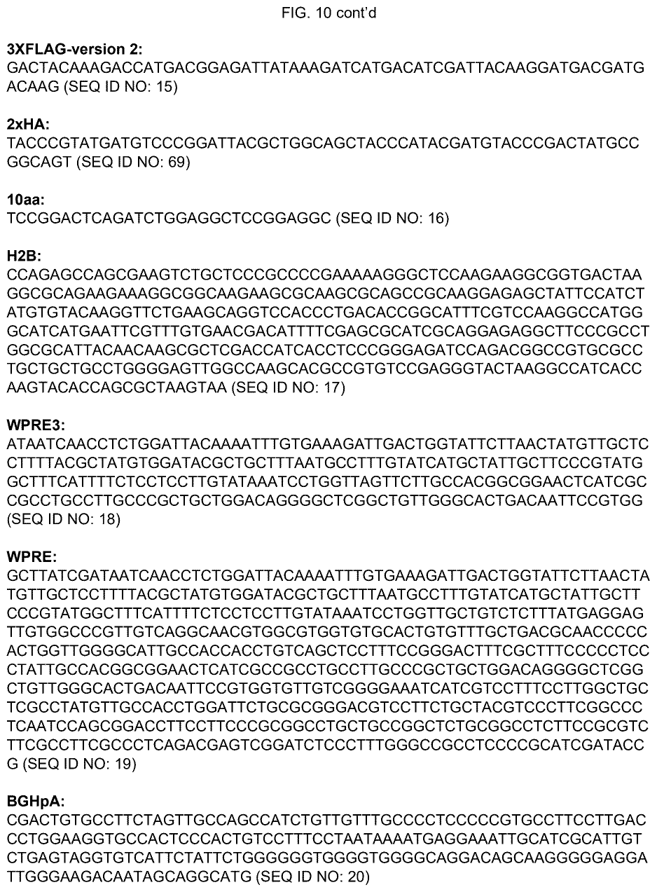

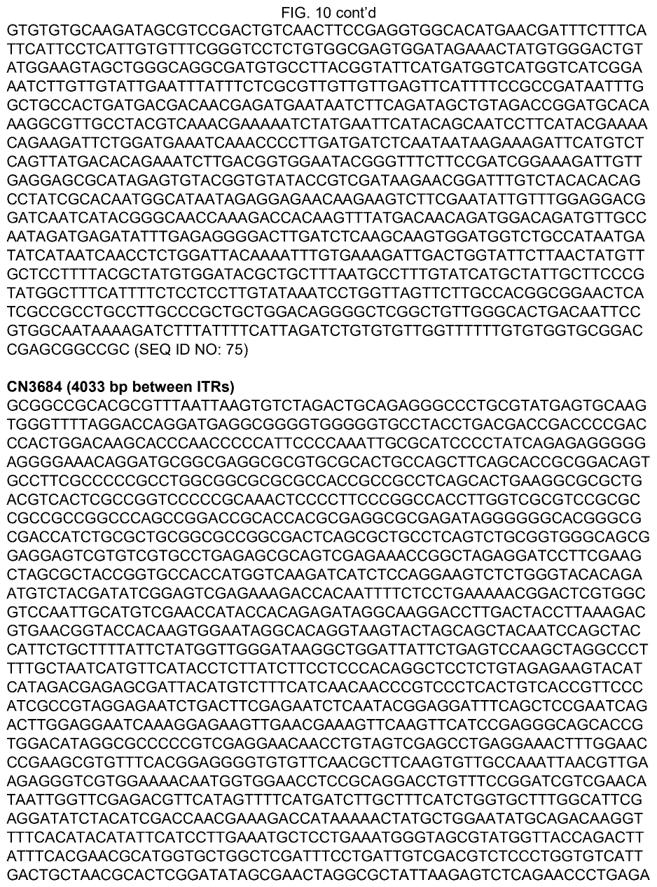

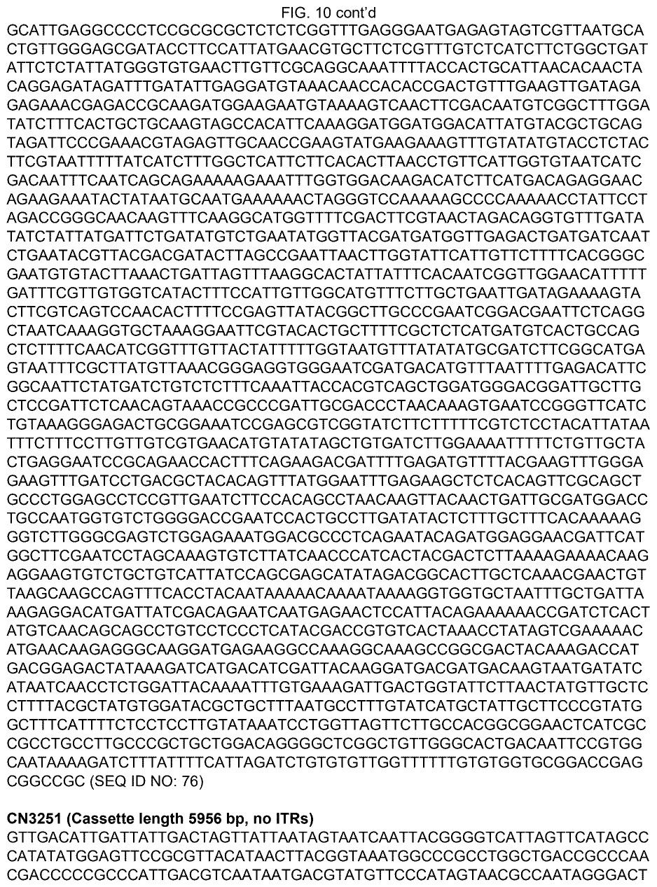

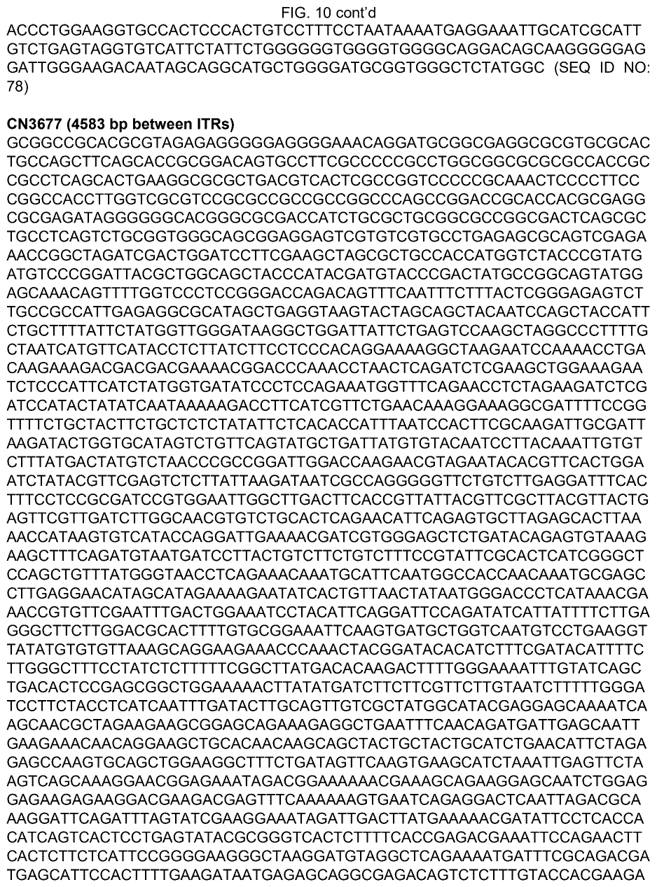

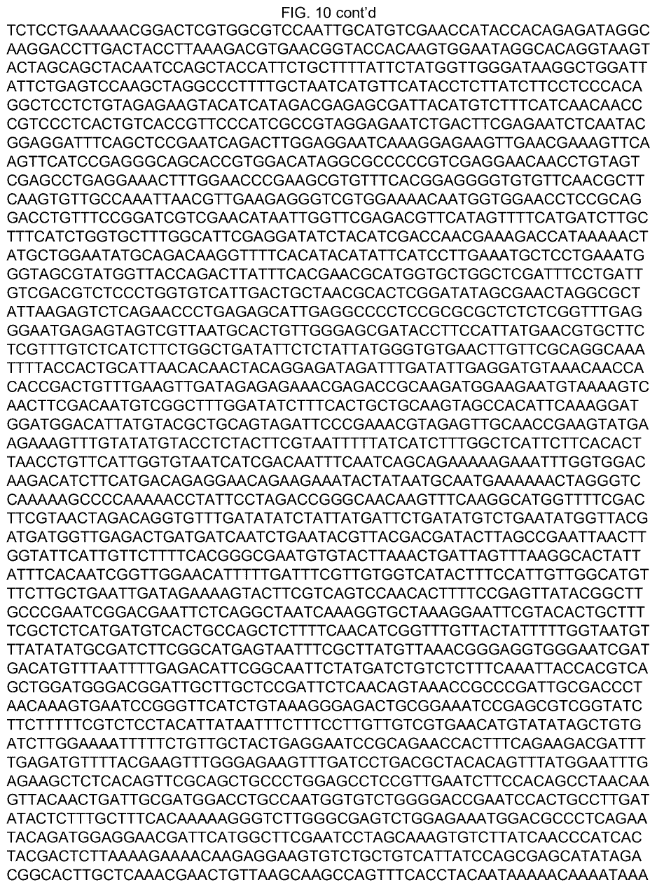

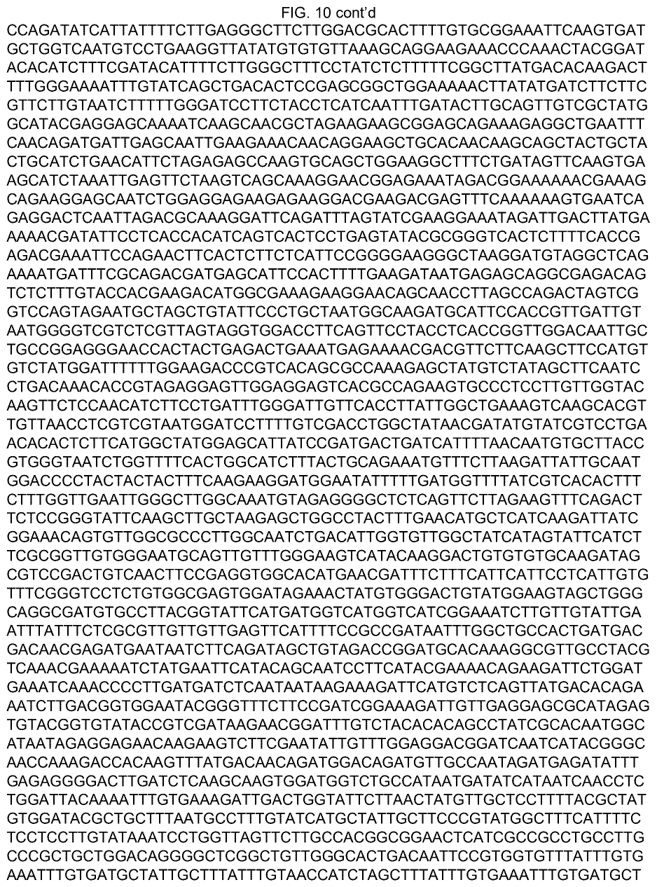

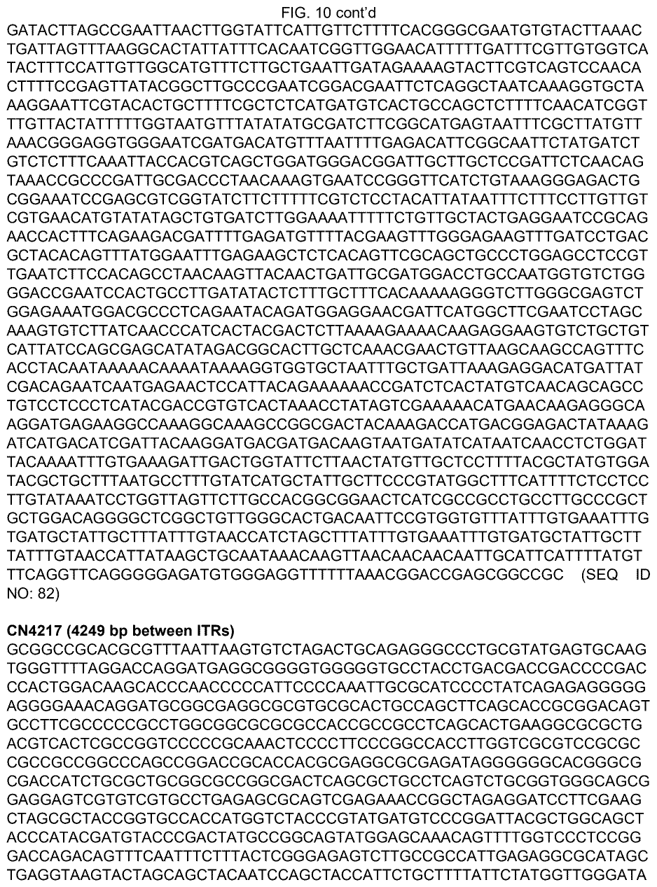

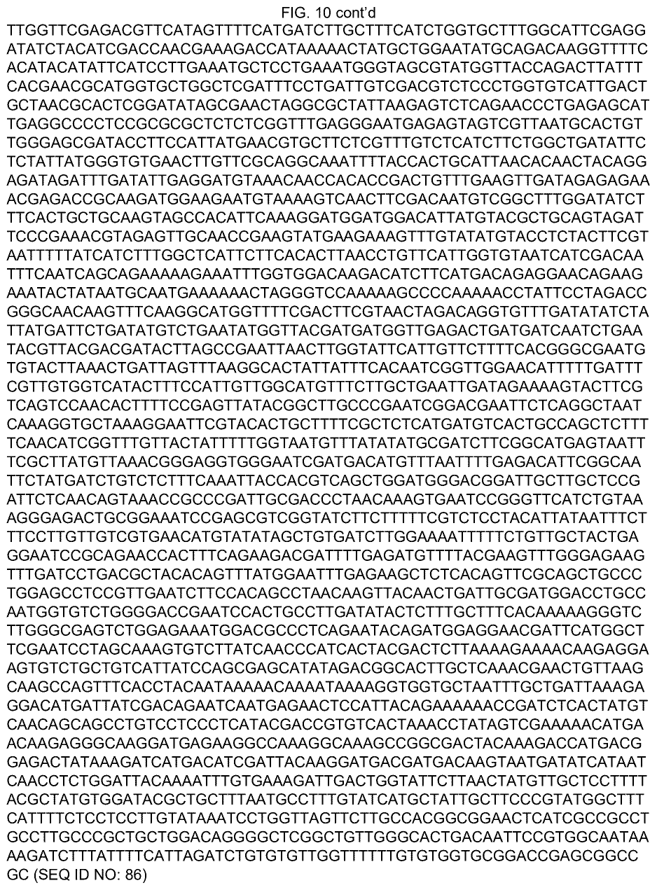

### DETAILED DESCRIPTION

The current disclosure provides expression constructs that result in unexpectedly rapid and high levels of protein expression within targeted central nervous system cell types for the purpose of rescuing voltage-gated sodium channel function. The expression constructs can be used to reverse or ameliorate the effects of voltage-gated sodium channel dysfunction in targeted cell types due to SCN1A variants that cause a disorder. Typically, SCN1A is difficult to deliver because of its large size which is greater than the packaging capacity of an adeno-associated virus (AAV). As described herein, the SCN1A coding sequence is thus packaged into two or more artificial expression constructs with sequences encoding split intein N- and C-fragments, wherein the expression product of the two or more artificial expression constructs are spliced to result in a full SCN1A coded protein and the split intein.

In particular embodiments, the current disclosure provides systems and methods to express the SCN1A coding sequence in a subject in need thereof. In particular embodiments, the system includes at least a first and a second artificial expression construct, wherein the first artificial expression construct includes a first portion of the SCN1A coding sequence, an N-intein coding sequence, and a first promoter sequence, wherein the N-intein coding sequence is located at the 3′ end of the first portion of the SCN1A coding sequence; and the second artificial expression construct includes a second portion of the SCN1A coding sequence, a C-intein coding sequence, and a second promoter sequence, wherein the C-intein coding sequence is located at the 5′ end of the second portion of the SCN1A coding sequence. In particular embodiments, when the first artificial expression construct and the second artificial expression construct are both expressed in a cell, the protein product of the SCN1A coding sequence is produced by protein splicing. In particular embodiments, the first and/or second artificial expression construct includes an enhancer that leads to targeted expression of the first and/or second portion of the SCN1A coding sequence within a targeted central nervous system cell type.

In particular embodiments, the current disclosure provides treatment of SCN1A-related disorders such as Dravet syndrome, myoclonic seizures, and intractable childhood epilepsy with generalized tonic-clonic seizures. For example, as disclosed herein, administration of the artificial expression constructs results in therapeutic efficacy to treat thermal seizures, myoclonic seizures, and generalized tonic-clonic seizures in a well-established in vivo mouse model of the disease.

In particular embodiments, the treatment includes the administration of at least a first and a second artificial expression construct to express the SCN1A coding sequence in a subject in need thereof. In particular embodiments, the first and second artificial expression construct each include a portion of the SCN1A coding sequence, a split intein coding sequence, and a promoter sequence.

Particular embodiments use split inteins including Cfa-N and Cfa-C. In particular embodiments, the Cfa-N intein coding sequence includes SEQ ID NO: 57. In particular embodiments, the Cfa-C intein coding sequence includes SEQ ID NO: 58.

In particular embodiments, the first portion of the SCN1A coding sequence and the second portion of the SCN1A coding sequence, when expressed and spliced together form a mature protein that rescues voltage-gated sodium channel function in a cell. The artificial expression construct can also include other regulatory elements if necessary or beneficial.

In particular embodiments, the artificial expression constructs are expressed in all neurons. In particular embodiments, the artificial expression constructs include an hSyn1 promoter and are expressed in neurons. In particular embodiments, the artificial expression constructs are expressed in all cell lines. In particular embodiments, the artificial expression constructs include a CMV promoter and are expressed in cell lines.

In particular embodiments, an enhancer is used to drive gene expression in a targeted central nervous system cell population. Particular embodiments of the artificial expression constructs utilize the following enhancers to drive gene expression within targeted central nervous system cell populations as follows (enhancer/targeted cell population): DLX2.0/forebrain GABAergic; hSyn1 with 4×2C or 8×2C miR binding site/pan-GABAergic neurons; and eHGT_078h/forebrain glutamatergic neurons. In particular embodiments, the artificial expression construct can include a shortened promoter or a minimal promoter. In particular embodiments, the shortened promoter includes the shortened hSyn1 promoter. In particular embodiments, the minimal promoter includes minBglobin.

Particular embodiments provide artificial expression construct pairs including the features of vectors described herein including vectors: CN3252 and CN3254, CN3683 and CN3684, CN3251 and CN3253, CN3677 and CN3678, CN4541 and CN4542, CN4217 and CN4218, or CN4642 and CN4643.

Aspects of the disclosure are now described with the following additional options and detail: (i) Artificial Expression Constructs & Vectors for Targeted Expression of Genes in Targeted Cell Types; (ii) Compositions for Administration (iii) Cell Lines Including Artificial Expression Constructs; (iv) Transgenic Animals; (v) Methods of Use; (vi) Kits and Commercial Packages; (vii) Exemplary Embodiments; and (viii) Closing Paragraphs. These headings are provided for organization purposes only and do not limit the scope or interpretation of the disclosure.

(i) Artificial Expression Constructs & Vectors for Targeted Expression of Genes in Targeted Cell Types. Systems and methods disclosed herein include the administration of at least a first and a second artificial expression construct to express the SCN1A coding sequence in a subject in need thereof. In particular embodiments, the first artificial expression construct includes a first portion of the SCN1A coding sequence, an N-intein coding sequence, and a first promoter sequence, wherein the N-intein coding sequence is located at the 3′ end of the first portion of the SCN1A coding sequence; and the second artificial expression construct includes a second portion of the SCN1A coding sequence, a C-intein coding sequence, and a second promoter sequence, wherein the C-intein coding sequence is located at the 5′ end of the second portion of the SCN1A coding sequence. In particular embodiments, when the first artificial expression construct and the second artificial expression construct are both expressed in a cell, the protein product of the SCN1A coding sequence is produced by protein splicing. In particular embodiments, the N-terminal portion of the coding sequence and the C-terminal portion of the coding sequence, when expressed and spliced together form a mature protein that rescues voltage-gated sodium channel function in a cell. The artificial expression construct can also include other regulatory elements if necessary or beneficial.

Inteins are a class of autocatalytic enzymes that contain both protease and ligase activities. Inteins are internal protein elements that self-excise from their host protein and catalyze ligation of the flanking sequences (exteins) with a peptide bond. Intein excision is a posttranslational process that does not require auxiliary enzymes or cofactors. This self-excision process is called “protein splicing,” by analogy to the splicing of RNA introns from pre-mRNA (Perler F et al, Nucl Acids Res. 22: 1125-1127 (1994)). The segments are called “intein” for internal protein sequence, and “extein” for external protein sequence, with upstream exteins termed “N-exteins” and downstream exteins called “C-exteins.” The products of the protein splicing process are two stable proteins: the mature protein and the intein.

Known inteins share a low degree of sequence similarity, with conserved residues only at the N- and C-termini. Most inteins begin with Ser or Cys and end in His-Asn or in His-Gln. The first amino acid of the C-extein is an invariant Ser, Thr, or Cys, but the residue preceding the intein at the N-extein is not conserved (Perler F. 2002, Nucl. Acids Res. 30: 383-384). However, residues proximal to the intein-splicing junction at both the N- and C-terminal exteins were recently found to accelerate or attenuate protein splicing (Amitai G et al. 2009, Proc. Natl. Acad. Sci. USA. 106: 11005-11010).

One class of inteins, termed “split inteins,” involves two complementary half inteins, termed the N-intein and C-intein, that associate selectively and extremely tightly to form an active intein enzyme (Shah N. H., et al, J. Amer. Chem. Soc. 135: 18673-18681; Dassa B., et al, Nucl. Acids Res., 37:2560-2573 (2009)). The two fragments of the split intein are encoded by two separately transcribed and translated genes. These so-called split inteins self-associate and catalyze protein-splicing activity in trans. Split inteins have been identified in diverse cyanobacteria and archaea (Caspi et al, Mol Microbiol. 50: 1569-1577 (2003); Choi J. et al, J Mol Biol. 556: 1093-1106 (2006.); Dassa B. et al, Biochemistry. 46:322-330 (2007.); Liu X. and Yang J., J Biol Chem. 275:26315-26318 (2003); Wu H. et al, Proc Natl Acad Sci USA. £5:9226-9231 (1998.); and Zettler J. et al, FEBS Letters. 553:909-914 (2009)), but have not been found in eukaryotes thus far. Recently, a bioinformatic analysis of environmental metagenomic data revealed 26 different loci with a novel genomic arrangement. At each locus, a conserved enzyme coding region is interrupted by a split intein, with a freestanding endonuclease gene inserted between the sections coding for intein subdomains. Among them, five loci were completely assembled: DNA helicases (gp41-I, gp41-8); Inosine-5′-monophosphate dehydrogenase (IMPDH-1); and Ribonucleotide reductase catalytic subunits (NrdA-2 and NrdJ-1). This fractured gene organization appears to be present mainly in phages (Dassa et al, Nucleic Acids Research. 57:2560-2573 (2009)).

More than 350 types of inteins are recognized at present, each of which can have differing rates of catalyzing a splicing reaction. The nomenclature of inteins is based on the scientific name of the organism to which it is found. Ssp inteins, for example, were first isolated from Synechocystis spp, whilst faster splicing Npu inteins were first isolated from Nostoc punctiforme. A database including a list of some of the known inteins can be found at http://www.biocenter.helsinki.fi/bi/iwaiAnBase/tools.neb.com/inbase/list.html.

In particular embodiments, the intein includes a Cfa intein, an Ssp intein, a gp41-1 intein, IMPDH-1 intein, Nrdj-1 intein, gp41-8 intein, or an Npu intein. In particular embodiments, the intein is functionally similar to a Cfa intein. In particular embodiments, the intein includes a Cfa intein. Herein, functionally similar to a Cfa intein means that the expression construct includes a variant of a Cfa intein, yet still results in construction of a functional protein (e.g., voltage-gated sodium channel). In particular embodiments, the intein includes an N-intein and a C-intein that when expressed and spliced together form an intein. For example, a Cfa intein includes a Cfa-N and a Cfa-C. In particular embodiments, one or more inteins can be used in each artificial expression construct. In particular embodiments, a mature protein can be expressed by splitting the coding sequence into two fragments and putting the N-terminal portion of the coding sequence with an N-intein into a first artificial expression construct and putting the C-terminal portion of the coding sequence with a C-intein into a second artificial expression construct and administering the first and second artificial expression construct to a cell. In particular embodiments, a mature protein can be expressed by splitting the coding sequence into three fragments and putting the N-terminal portion of the coding sequence with a first N-intein into a first artificial expression construct, putting the middle portion of the coding sequence with a first C-intein and second N-intein into a second artificial expression construct, and putting the C-terminal portion of the coding sequence with a second C-intein into a second artificial expression construct, wherein the first N-intein and first C-intein specifically splice together to form an intein and the second N-intein and second C-intein specifically splice together to form an intein, and administering the first, second, and third artificial expression construct to a cell. Similarly, mature proteins can be formed from several fragments using the appropriate number of inteins. In particular embodiments, the Cfa-N intein coding sequence includes SEQ ID NO: 57. In particular embodiments, the Cfa-C intein coding sequence includes SEQ ID NO: 58.

In particular embodiments, artificial expression constructs include regulatory elements that drive gene expression in a targeted or not targeted cell population. Particular examples of regulatory elements utilized within artificial expression constructs disclosed herein include DLX2.0, minBglobin promoter, hSyn1 promoter, CMV promoter, shortened hSyn1 promoter, 4×2C miR binding site, 8×2c miR binding site, and eHGT_078h.

In particular embodiments, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences.

In particular embodiments, a targeted central nervous system cell type enhancer is an enhancer that is uniquely or predominantly utilized by the targeted central nervous system cell type. A targeted central nervous system cell type enhancer enhances expression of a gene in the targeted central nervous system. In certain embodiments, a targeted central nervous system cell type enhancer is also a targeted central nervous system type enhancer that enhances expression of a gene in the targeted central nervous system and does not substantially direct expression of genes in other non-targeted cell types, thus having cell type-specific transcriptional activity.

In particular embodiments, regulatory elements can include miRNA-guided neuron tags (e.g., mAGNETs), herein referred to as miR binding sites. Exemplary miR binding sites include 4×2C, 4×3C, and 8×2C. In particular embodiments, 4×2C includes the sequences as set forth in SEQ ID NO: 56. Additional miR binding sites include 4×Mir128_4×Mir221_BPL, 4×Mir128_4×Mir221_4×Mir183_4×Mir122_BPL, and 4×Mir183_4×Mir122_BPL.

When a heterologous coding sequence operatively linked to an enhancer disclosed herein leads to expression in a targeted cell type, it leads to expression of the administered heterologous coding sequence in the intended cell type.

When a heterologous coding sequence is selectively expressed in selected cells, it leads to expression of the administered heterologous coding sequence in the intended cell type and is not substantially expressed in other cell types, as explained in additional detail below. In particular embodiments, not substantially expressed in other cell types is less than 50% expression in a reference cell type as compared to a targeted cell type; less than 40% expression in a reference cell type as compared to a targeted cell type; less than 30% expression in a reference cell type as compared to a targeted cell type; less than 20% expression in a reference cell type as compared to a targeted cell type; or less than 10% expression in a reference cell type as compared to a targeted cell type. In particular embodiments, a reference cell type refers to non-targeted cells. The non-targeted cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted cell with a different gene expression profile than the targeted cells.

In particular embodiments, the product of the coding sequence may be expressed at low levels in non-selected cell types, for example at less than 1% or 1%, 2%, 3%, 5%, 10%, 15% or 20% of the levels at which the product is expressed in selected cells. In particular embodiments, the targeted central nervous system cell type is the only cell type that expresses the right combination of transcription factors that bind an enhancer disclosed herein to drive gene expression. Thus, in particular embodiments, expression occurs exclusively within the targeted cell type.

In particular embodiments, targeted cell types (e.g., neuronal, and/or non-neuronal) can be identified based on transcriptional profiles, such as those described in Tasic et al., Nature. 563, 72-78 (2018) and Hodge et al., Nature. 573, 61-68 (2019). For reference, the following description of cell types and distinguishing features is also provided:

### Neocortical GABAergic Neuron Subclasses:

All: Express GABA synthesis genes Gad1/GAD1 and Gad2/GAD2.
Lamp5, Sncg, Serpinf1, and Vip GABAergic neurons: Developmentally derived from neuronal progenitors from the caudal ganglionic eminence (CGE) or preoptic area (POA).
Sst and Pvalb GABAergic neurons: Developmentally derived from neuronal progenitors in the medial ganglionic eminence (MGE).
Lamp5 GABAergic neurons: Found in many neocortical layers, especially upper (L1-L2/3), and have mainly neurogliaform and single bouquet morphology.
Lamp5_Lhx6 GABAergic neurons: A subset of Lamp5 GABAergic neurons that co-express Lamp5 and Lhx6.
Sncg GABAergic neurons: Found in many neocortical layers, and have molecular overlaps with Lamp5 and Vip cells, but inconsistent expression of Lamp5 or vasoactive intestinal peptide (Vip), with more consistent expression of Sncg.
Serpinf1 GABAergic neurons: Found in many neocortical layers, and have molecular overlaps with Sncg and Vip cells, but inconsistent expression of Sncg or Vip, with more consistent expression of Serpinf1.
Vip GABAergic neurons: Found in many neocortical layers, but especially frequent in upper layers (L1-L4), and highly express the neurotransmitter Vip.
Sst GABAergic neurons: Found in many neocortical layers, but especially frequent in lower layers (L5-L6). They highly express the neurotransmitter somatostatin (Sst), and frequently block dendritic inputs to postsynaptic neurons. Included in this subclass are sleep-active Sst ChodI neurons (which also express Nos1 and Tacr1) that are highly distinct from other Sst neurons but express some shared marker genes including Sst. In human, SST gene expression is often detected in layer 1 LAMP5+ GABAergic neuron subtypes.
Pvalb GABAergic neurons: Found in many neocortical layers, but especially frequent in lower layers (L5-L6). They highly express the calcium-binding protein parvalbumin (Pvalb), express neuropeptide Tac1, and frequently dampen the output of postsynaptic neurons. Most fast-spiking GABAergic neurons express Pvalb strongly. Included in this subclass are chandelier cells, which have distinct, chandelier-like morphology and express the markers Cpne5 and Vipr2 in mouse, and NOG and UNC5B in human.
Meis2: A distinct subclass defined by a single type, only neocortical GABAergic neuron type that expresses Meis2 gene, and does not express some other genes that are expressed by all other neocortical GABAergic neuron types (for example, Thy1 and Scn2b). This type is found in L6b and subcortical white matter.

### Neocortical Glutamatergic Neuron Subclasses:

All: Express glutamate transmitters Slc17a6 and/or Slc17a7. They all express Snap25 and lack expression of Gad1/Gad2.
L2/3 IT glutamatergic neurons: Primarily reside in Layer 2/3 and have mainly intratelencephalic (cortico-cortical) projections.
L4 IT glutamatergic neurons: Primarily reside in Layer 4 and mainly have either local or intratelencephalic (cortico-cortical) projections.
L5 IT glutamatergic neurons: Primarily reside in Layer 5 and have mainly intratelencephalic (cortico-cortical) projections. Also called L5a.
L5 PT glutamatergic neurons: Primarily reside in Layer 5 and have mainly cortico-subcortical (pyramidal tract or corticofugal) projections. Also called L5b or L5 CF (corticofugal) or L5 ET (extratelencephalic). This subclass includes cells that are located in the primary motor cortex and neighboring areas and are corticospinal projection neurons, which are associated with motor neuron/movement disorders, such as ALS. This subclass includes thick-tufted pyramidal neurons, including distinctive subtypes found only in specialized regions, e.g., Betz cells, Meynert cells, and von Economo cells.
L5 NP glutamatergic neurons: Primarily reside in Layer 5 and have mainly nearby projections.
L6 CT glutamatergic neurons: Primarily reside in Layer 6 and have mainly cortico-thalamic projections.
L6 IT glutamatergic neurons: Primarily reside in Layer 6 and have mainly intratelencephalic (cortico-cortical) projections.
L6 IT Car3 glutamatergic neurons: Most densely present in claustrum and endopyriform nucleus, and sparsely throughout L6 in many cortical areas including the primary visual cortex. These cells have mainly intratelencephalic (cortico-cortical) projections. Additional marker genes for claustrum enriched neurons include Gnb4 and Ntng2.
L6b glutamatergic neurons: Primarily reside in the neocortical subplate (L6b), with local (near the cell body) projections and some cortico-cortical projections from VISp to anterior cingulate, and cortico-subcortical projections to the thalamus.
CR neurons: A distinct subclass defined by a single type in L1, Cajal-Retzius cells express distinct molecular markers Lhx5 and Trp73.

### Thalamus GABAergic Neuron Classes and Subclasses:

All: Express GABA synthesis genes Gad1/GAD1 and/or Gad2/GAD2.
Thalamic reticular nucleus (TRN) neurons: Express GABA synthesis genes Gad1/GAD1 and Pvalb/PVALB.

### Thalamus Glutamatergic Neuron Classes and Subclasses:

All glutamatergic neurons: Express glutamate transporters Slc17a6/SLC17A6 and/or Slc17a7/SLC17A7. They lack expression of Gad1/Gad2 and have expression of one or more of the marker genes Synpo2/SYNPO2, Rgs16/RGS16, Plekhg1/PLEKHG1, and Prkcd/PRKCD.
Glutamatergic neurons within the parafascicular (Pf) nuclei: The Pf nuclei is a posterior component of the intralaminar nuclei of the thalamus. The Pf nuclei plays a role in the feedback systems of basal ganglia-thalamo-cortical circuits critically involved in cognitive processes.

### Targeted Striatal Cell Classes:

The striatum (Str) is important in translating cortical activity into voluntary motor actions. Regarding the striatum, corresponding structures in human/primate are called the putamen, caudate, and ventral striatum containing the nucleus accumbens. In rodent, the striatum includes the dorsal striatum plus the nucleus accumbens. Thus, in human/primate, the putamen and caudate collectively are the equivalent of the rodent dorsal striatum.

Striatal cell Classes and Subclasses:
Medium spiny neurons, pan: include 95% of striatal neurons and known to express GABA synthesis genes Gad1/GAD1 and Gad2/GAD2, as well as Ppp1r1b/PPP1R1B. Medium spiny neurons expressing Drd3 are herein referred to as Drd3+ medium spiny neurons.
Medium spiny neurons, direct pathway-projecting: include nearly 50% of striatal neurons and are enriched for expression of Drd1/DRD1, Pdyn/PDYN, and Slc35d3/SLC35D3. The major axon projection from direct pathway medium spiny neurons is to the substantia nigra pars reticulata (SNr) or to the inner division of the globus pallidus (GPi).
Medium spiny neurons, indirect pathway-projecting: include nearly 50% of striatal neurons and are enriched for expression of Drd2/DRD2, Adora2a/ADORA2A, Gpr6/GPR6, and Penk/PENK. The major axon projection from indirect pathway medium spiny neurons is to the external segment of the globus pallidus (GPe).
Striatal interneuron-cholinergic: A rare interneuron population including 1% of striatal neurons. These local interneurons have large somata and aspiny dendrites and are known to express Chat/CHAT and release the neurotransmitter acetylcholine.

The cerebellum is located at the posterior of the brain. The cerebellum processes inputs from the cerebral motor cortex, different brainstem nuclei, and sensory receptors. Two types of neurons play major roles in the cerebellar circuit, including Purkinje cells and granule cells. The cerebellum also receives dopaminergic, serotonergic, noradrenergic, and cholinergic inputs.
Cerebellar Purkinje cells: large GABAergic neurons that are the only projection neurons and the sole output from the cerebellum. Their cell bodies form a single layer, so called ‘Purkinje cell layer’, and they express parvalbumin.
Deep cerebellar nucleus neurons: neurons located in the deep cerebellar nuclei structures. These include glutamatergic and GABAergic cells that express the gene Pvalb.
Molecular layer interneurons (MLI): neurons within the cerebellum that participate in spatially structured networks via chemical and electrical synapses.
Chandelier cells are a specialized GABAergic interneuron that selectively innervates pyramidal neurons.
Striatal medium spiny neurons are the major striatal neuron. They receive synaptic input from both glutamatergic and dopaminergic afferents.

Dopaminergic neurons secrete dopamine. Dopaminergic neurons of the midbrain are the main source of dopamine in the mammalian central nervous system. The loss of dopaminergic neurons is associated with one of the most prominent human neurological disorders, Parkinson's disease. Studies into the developmental pathways which are involved in the generation of dopaminergic neurons in the brain have led to the identification of several specific transcription factors including Nurr1, Lmx1b and Pitx3, all shown to be important in the development of the mesencephalic dopaminergic system.

In particular embodiments, a coding sequence is a heterologous coding sequence that encodes an effector element. An effector element is a sequence that is expressed to achieve, and that in fact achieves, an intended effect. Examples of effector elements include reporter genes/proteins and functional genes/proteins. In particular embodiments, the effector element is a protein that rescues voltage-gated sodium channel function. In particular embodiments, the coding sequence is SCN1A.

In particular embodiments, artificial expression constructs can deliver SCN1A as several fragments of SCN1A delivered by several artificial expression constructs. For example, SCN1A can be delivered in a first artificial expression construct including a first portion of the SCN1A coding sequence and second artificial expression construct including a second portion of the SCN1A coding sequence. In particular embodiments, the first portion of the SCN1A coding sequence is the N-terminal portion of the coding sequence and the second portion of the SCN1A coding sequence is the C-terminal portion of the coding sequence. The SCN1A coding sequence can be split into an N-terminal portion and C-terminal portion at any point such that upon intein fusion, a functional SCN1A molecule is expressed. In particular embodiments, an N-terminal portion of the SCN1A coding sequence includes hSCN1A-CO-Nterm1049 (SEQ ID NO: 59), hSCN1A-CO-Nterm956 (SEQ ID NO: 61), or hSCN1A-CO-Nterm947 (SEQ ID NO: 63). In particular embodiments, a C-terminal portion of the SCN1A coding sequence includes hSCN1A-CO-Cterm949 (SEQ ID NO: 60), hSCN1A-CO-Cterm1042 (SEQ ID NO: 62), or hSCN1A-CO-Cterm1051 (SEQ ID NO: 64).

Exemplary reporter genes/proteins include those expressed by Addgene ID #s 83894 (pAAV-hDIx-Flex-dTomato-Fishell_7), 83895 (pAAV-hDIx-Flex-GFP-Fishell_6), 83896 (pAAV-hDIx-GiDREADD-dTomato-Fishell-5), 83898 (pAAV-mDIx-ChR2-mCherry-Fishell-3), 83899 (pAAV-mDIx-GCaMP6f-Fishell-2), 83900 (pAAV-mDIx-GFP-Fishell-1), and 89897 (pcDNA3-FLAG-mTET2 (N500)). Exemplary reporter genes particularly can include those which encode an expressible fluorescent protein, or expressible biotin; blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanI, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenI, Oregon Green™ (Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedI, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowI); and tandem conjugates.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10²¹ m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

In particular embodiments, artificial expression constructs can include DNA and RNA editing tools such CRISPR/Cas (e.g., guide RNA and a nuclease, such as Cas, Cas9 or cpf1). Functional molecules can also include engineered Cpf1s such as those described in US 2018/0030425, US 2016/0208243, WO/2017/184768 and Zetsche et al. (2015) Cell 163: 759-771; single gRNA (see e.g., Jinek et al. (2012) Science 337:816-821; Jinek et al. (2013) eLife 2:e00471; Segal (2013) eLife 2:e00563) or editase, guide RNA molecules, microRNA, or homologous recombination donor cassettes.

In particular embodiments, artificial expression constructs can include a localizing cassette. In particular embodiments, localizing cassettes are used to localize a molecule (e.g., a vector, a protein, a sensor) to a specific subcellular compartment such as the soma, axon, or dendrite(s) of a neuron. In particular embodiments, localizing cassettes include a soma tag (e.g., soma (EE-RR)) to localize at the soma; an axon tag (e.g., derived from GAP43) or synaptophysin (sy) to localize at the axon; hydrophobic tails to localize at the plasma membrane; and hydrophobicity or alkyl chain to localize at the endoplasmic reticulum. In particular embodiments, localizing cassettes are fused to a sensor molecule such as a GECI. In particular embodiments, fusion proteins of a GECI and a localizing cassette includes soma-jGCaMP8s, axon-jRGECO1a, syGCaMP5G, and soma-jGCaMP7s. Artificial expression constructs can encode nuclear localization proteins, such as Histone H1, Histone H2A, Histone H2B, Histone H3, Histone H4, histone-like protein HPhA, or H2B*.

In particular embodiments, artificial expression constructs can include tag cassettes. A tag cassette includes His tag (HHHHHH; SEQ ID NO: 34), Flag tag (DYKDDDDK; SEQ ID NO: 35), Xpress tag (DLYDDDDK; SEQ ID NO: 36), Avi tag (GLNDIFEAQKIEWHE; SEQ ID NO: 37), Calmodulin tag (KRRWKKNFIAVSAANRFKKISSSGAL; SEQ ID NO: 38), Polyglutamate tag, HA tag (YPYDVPDYA; SEQ ID NO: 39), Myc tag (EQKLISEEDL; SEQ ID NO: 40), Strep tag (which refers the original STREP® tag (WRHPQFGG; SEQ ID NO: 41), STREP® tag II (WSHPQFEK SEQ ID NO: 42 (IBA Institut fur Bioanalytik, Germany); see, e.g., U.S. Pat. No. 7,981,632), Softag 1 (SLAELLNAGLGGS; SEQ ID NO: 43), Softag 3 (TQDPSRVG; SEQ ID NO: 44), and V5 tag (GKPIPNPLLGLDST; SEQ ID NO: 45). In particular embodiments, a tag cassette includes a fusion of tag cassettes such as 3×FLAG. In particular embodiments, 3×FLAG includes the sequence set forth in SEQ ID NO: 15.

Additional effector elements include Cre, iCre, dgCre, FlpO, and tTA2. iCre refers to a codon-improved Cre. dgCre refers to an enhanced GFP/Cre recombinase fusion gene with an N terminal fusion of the first 159 amino acids of the Escherichia coli K-12 strain chromosomal dihydrofolate reductase gene (DHFR or folA) harboring a G67S mutation and modified to also include the R12Y/Y100I destabilizing domain mutation. FlpO refers to a codon-optimized form of FLPe that greatly increases protein expression and FRT recombination efficiency in mouse cells. Like the Cre/LoxP system, the FLP/FRT system has been widely used for gene expression (and generating conditional knockout mice, mediated by the FLP/FRT system). tTA2 refers to tetracycline transactivator.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the artificial expression constructs include an internal ribosome entry site (IRES) sequence. IRES allow ribosomes to initiate translation at a second internal site on a mRNA molecule, leading to production of two proteins from one mRNA. In particular embodiments, IRES includes IRES2. In particular embodiments, IRES2 allows for a second protein open reading frame (ORF) to be translated from the same transcript. This is unlike the 2A sequence which allows for a single ORF to be cleaved into two proteins, with similar efficiencies of production.

Coding sequences encoding molecules (e.g., RNA, proteins) described herein can be obtained from publicly available databases and publications. Coding sequences can further include various sequence polymorphisms, mutations, and/or sequence variants wherein such alterations do not affect the function of the encoded molecule. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, insulators, and/or post-regulatory elements, such as termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin (also referred to as minBGprom), CMV promoter, hSyn1 promoter, shortened hSyn1 promoter, minCMV, minCMV* (minCMV* is minCMV with a SacI restriction site removed), minRho, minRho* (minRho* is minRho with a SacI restriction site removed), SV40 immediately early promoter, the Hsp68 minimal promoter (proHSP68), and the Rous Sarcoma Virus (RSV) long-terminal repeat (LTR) promoter. Minimal promoters have no activity to drive gene expression on their own but can be activated to drive gene expression when linked to a proximal enhancer element.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived components that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus vectors refer to those constructs containing adenovirus sequences sufficient to (a) support packaging of an artificial expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in targeted cell populations. scAAV refers to a self-complementary AAV. pAAV refers to a plasmid adeno-associated virus. rAAV refers to a recombinant adeno-associated virus. pSMART-HCKan is a high copy number vector with a kanamycin resistance marker for efficient blunt cloning of unstable sequences.

Other viral vectors may also be employed. For example, vectors derived from viruses such as vaccinia virus, polioviruses and herpes viruses may be employed. They offer several attractive features for various mammalian cells.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and US regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al., 1999, J. Virol., 73:2886); the posttranscriptional regulatory element present in hepatitis B virus (HPRE) (Smith et al., Nucleic Acids Res. 26(21):4818-4827, 1998); and the like (Liu et al., Genes Dev., 9:1766, 1995). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation signal 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA, hGHpA, SV40 pA, or shortPolyA. In particular embodiments, a preferred embodiment of an expression construct includes a terminator element. These elements can serve to enhance transcript levels and to minimize read through from the construct into other plasmid sequences.

In particular embodiments, a viral vector further includes one or more insulator elements. Insulators elements may contribute to protecting viral vector-expressed sequences, e.g., effector elements or expressible elements, from integration site effects, which may be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). In particular embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken β-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Particular embodiments of vectors disclosed herein include:

| Expression | |
|---|---|
| Construct | |
| Name | Features |
| CN3252 | pAAV-DLX2.0-minBglobin-2xHA-hSCN1A-CO-Nterm1049-Intron-CfaN-WPRE3-SV40pA |
| CN3254 | pAAV-DLX2.0-minBglobin-CfaC-hSCN1A-CO-Cterm949-Intron-3xFLAG-WPRE3-SV40pA |
| CN3683 | pAAV-hSyn1-2xHA-hSCN1A-CO-Nterm1049-Intron-CfaN-WPRE3-shortPolyA |
| CN3684 | pAAV-hSyn1-CfaC-hSCN1A-CO-Cterm949-Intron-3xFLAG-WPRE3-shortPolyA |
| CN3251 | pSMART-HCKan-CMVpromoter-2xHA-hSCN1A-CO-Nterm1049-Intron-CfaN_IRES2_SYFP2_BGHpA |
| CN3253 | pSMART-HCKan-CMVpromoter-CfaC-hSCN1A-CO-Cterm949-Intron-3xFLAG_IRES2_mScarlet-3xNLS_BGHpA |
| CN3677 | pAAV-hSyn1short-2xHA-hSCN1A-CO-Nterm1049-Intron-CfaN-4x2C-WPRE3-shortPolyA |
| CN3678 | pAAV-hSyn1short-CfaC-hSCN1A-CO-Cterm949-Intron-3xFLAG-4x2C-WPRE3-shortPolyA |
| CN4541 | pAAV-eHGT_078h-minBglobin-2xHA-hSCN1A-CO-Nterm1049-Intron-CfaN-WPRE3-SV40pA |
| CN4542 | pAAV-eHGT_078h-minBglobin-CfaC-hSCN1A-CO-Cterm949-Intron-3xFLAG-WPRE3-SV40pA |
| CN4217 | pAAV-hSyn1-2xHA-hSCN1A-CO-Nterm956-Intron-CfaN-WPRE3-shortPolyA |
| CN4218 | pAAV-hSyn1-CfaC-hSCN1A-CO-Cterm1042-Intron-3xFLAG-WPRE3-shortPolyA |
| CN4642 | pAAV-hSyn1-2xHA-hSCN1A-CO-Nterm947-Intron-CfaN-WPRE3-shortPolyA |
| CN4643 | pAAV-hSyn1-CfaC-hSCN1A-CO-Cterm1051-Intron-3xFLAG-WPRE3-shortPolyA |

Subcomponent sequences within the larger vector sequences can be readily identified by one of ordinary skill in the art and based on the contents of the current disclosure. Nucleotides between identifiable and enumerated subcomponents reflect restriction enzyme recognition sites used in assembly (cloning) of the constructs, and in some cases, additional nucleotides do not convey any identifiable function. These segments of complete vector sequences can be adjusted based on use of different cloning strategies and/or vectors. In general, short 6-nucleotide palindromic sequences reflect vector construction artifacts that are not important to vector function.

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh.10 (Yang, et al., Mol Ther. 2014; 22(7): 1299-1309), AAV1R6, AAV1R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1172), AAV-PHP.B (Deverman et al., Nat Biotechnol. 2016; 34(2): 204), AAV-PPS (Chen et al., Nat Med. 2009; 15: 1215), and PHP.eB. In particular embodiments, the PHP.eB capsid differs from AAV9 such that, using AAV9 as a reference, amino acids starting at residue 586: S-AQ-A (SEQ ID NO: 46) are changed to S-DGTLAVPFK-A (SEQ ID NO: 47). In particular embodiments, PHP.eb refers to SEQ ID NO: 30.

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31(4): 317), for example, as described in relation to clinical trials for the treatment of spinal muscular atrophy (SMA) syndrome by AveXis (AVXS-101, NCT03505099) and the treatment of CLN3 gene-Related Neuronal Ceroid-Lipofuscinosis (NCT03770572).

AAVrh.10, was originally isolated from rhesus macaques and shows low seropositivity in humans when compared with other common serotypes used for gene delivery applications (Selot et al., Front Pharmacol. 2017; 8: 441) and has been evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh.10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 48) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8(6): 609).

AAV-PHP.S (Addgene, Watertown, MA) is a variant of AAV9 generated with the CREATE method that encodes the 7-mer sequence QAVRTSL (SEQ ID NO: 49), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 50). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 51) epitope into the capsid of AAV2, shows a dramatically improved brain tropism relative to AAV2.

For additional information regarding capsids that cross the blood brain barrier, see Chan et al., Nat. Neurosci. 2017 August: 20(8): 1172-1179.

In particular embodiments, a capsid that results in transduction of targeted cell types in a primate following administration (e.g., I.V. administration) is chosen. In particular embodiments, a capsid that results in widespread transduction of tissue and cell types impacted by the loss of SCN1A following administration is chosen. In particular embodiments, targeted cell types are neurons. In particular embodiments, neurons include GABAergic neurons or glutamatergic neurons. In particular embodiments, GABAergic neurons include pan-GABAergic neurons, forebrain GABAergic neurons, hippocampal GABAergic neurons, or cortical GABAergic neurons. In particular embodiments, glutamatergic neurons include forebrain glutamatergic neurons

(ii) Compositions for Administration. Artificial expression constructs and vectors that result in rescue of voltage-gated sodium channel function of the present disclosure (referred to herein as physiologically active components) can be formulated with a carrier or more than one carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously.

In particular embodiments, compositions can be formulated for intravenous, intraparenchymal, intraocular, intravitreal, parenteral, subcutaneous, intracerebroventricular (ICV), intramuscular, intrathecal, intraspinal, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24(12):1113-1128, 1998; Quintanar-Guerrero et al., Pharm Res. 15(7):1056-1062, 1998; Quintanar-Guerrero et al., J. Microencapsul. 15(1):107-119, 1998; Douglas et al., Crit Rev Ther Drug Carrier Syst 3(3):233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2):199-202, 1980; Couvreur et al., Crit Rev Ther Drug Carrier Syst. 5(1)1-20, 1988; zur Muhlen et al., Eur J Pharm Biopharm, 45(2):149-155, 1998; Zambaux et al., J Control Release 50(1-3):31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17(1):33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

(iii) Cell Lines Including Artificial Expression Constructs. The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

A variety of host cell lines can be used, but in particular embodiments, the cell is a mammalian cell. In particular embodiments, the artificial expression construct includes a regulatory element and/or a vector sequence of DLX2.0, minBglobin promoter, hSyn1 promoter, CMV promoter, shortened hSyn1 promoter, 4×2C miR binding site, 8×2C miR binding site, eHGT_078h, Cfa-N, Cfa-C, hSCN1A-CO-Nterm1049, hSCN1A-CO-Cterm949, hSCN1A-CO-Nterm956, hSCN1A-CO-Cterm1042, hSCN1A-CO-Nterm947, and/or hSCN1A-CO-Cterm1051 and/or CN3252, CN3254, CN3683, CN3684, CN3251, CN3253, CN3677, CN3678, CN4541, CN4542, CN4217, CN4218, CN4642, or CN4643, and the cell line is a human, primate, or murine cell. Cell lines which can be utilized for transgenesis in the present disclosure also include primary cell lines derived from living tissue such as rat or mouse brains and organotypic cell cultures, including brain slices from animals such as rats, mice, non-human primates, or human neurosurgical tissue.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In particular embodiments, “neuronal” describes something that is of, related to, or includes, neuronal cells. Neuronal cells are defined by the presence of an axon and dendrites.

The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer sequence to be evaluated. (See for example Fiskerstrand et al., FEBS Lett 458: 171-174, 1999).

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4:1614-22. Bibel, et al., 2007, Nat. Protoc. 2:1034-43 describes a protocol to produce glutamatergic neurons from stem cells while Chatzi, et al., 2009, Exp. Neurol. 217:407-16 describes a procedure to produce GABAergic neurons. This procedure includes exposing stem cells to all-trans-RA for three days. After subsequent culture in serum-free neuronal induction medium including Neurobasal medium supplemented with B27, bFGF and EGF, 95% GABA neurons develop

U.S. Publication No. 2012/0329714 describes use of prolactin to increase neural stem cell numbers while U.S. Publication No. 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-α and TGF-β (U.S. Pat. Nos. 5,851,832 and 5,753,506).

In particular embodiments, yeast one-hybrid systems may also be used to identify compounds that inhibit specific protein/DNA interactions, such as transcription factors for DLX2.0, minBglobin promoter, hSyn1 promoter, CMV promoter, shortened hSyn1 promoter, 4×2C miR binding site, 8×2C miR binding site, and/or eHGT_078h.

Transgenic animals are described below. Cell lines may also be derived from such transgenic animals. For example, primary tissue culture from transgenic mice (e.g., also as described below) can provide cell lines with the artificial expression construct already integrated into the genome (for an example see MacKenzie & Quinn, Proc Natl Acad Sci USA 96: 15251-15255, 1999).

(iv) Transgenic Animals. Another aspect of the disclosure includes transgenic animals, the genome of which contains an artificial expression construct including DLX2.0, minBglobin promoter, hSyn1 promoter, CMV promoter, shortened hSyn1 promoter, 4×2C miR binding site, 8×2C miR binding site, eHGT_078h, Cfa-N, Cfa-C, hSCN1A-CO-Nterm1049, hSCN1A-CO-Cterm949, hSCN1A-CO-Nterm956, hSCN1A-CO-Cterm1042, hSCN1A-CO-Nterm947, and/or hSCN1A-CO-Cterm1051 operatively linked to a heterologous coding sequence. In particular embodiments, the genome of a transgenic animal includes CN3252 and CN3254, CN3683 and CN3684, CN3251 and CN3253, CN3677 and CN3678, CN4541 and CN4542, CN4217 and CN4218, and CN4642 and CN4643. In particular embodiments, when a non-integrating vector is utilized, a transgenic animal includes an artificial expression construct including DLX2.0, minBglobin promoter, hSyn1 promoter, CMV promoter, shortened hSyn1 promoter, 4×2C miR binding site, 8×2C miR binding site, eHGT_078h, Cfa-N, Cfa-C, hSCN1A-CO-Nterm1049, hSCN1A-CO-Cterm949, hSCN1A-CO-Nterm956, hSCN1A-CO-Cterm1042, hSCN1A-CO-Nterm947, and/or hSCN1A-CO-Cterm1051 and/or CN3252 and CN3254, CN3683 and CN3684, CN3251 and CN3253, CN3677 and CN3678, CN4541 and CN4542, CN4217 and CN4218, and CN4642 and CN4643 within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem cells using techniques known in the art. Typically, the artificial expression construct is introduced into cultured murine embryonic stem (ES) cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually, the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice crossed with an appropriate strain can produce offspring that will carry the transgene.

In addition to the methods of delivery described above, the following techniques are also contemplated as alternative methods of delivering artificial expression constructs to target cells or targeted tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17(1):33-58, 1998); transdermal matrices (U.S. Pat. Nos. 5,770,219 and 5,783,208); feedback-controlled delivery (U.S. Pat. No. 5,697,899), and any other delivery method available and/or described elsewhere in the disclosure.

(v) Methods of Use. In particular embodiments, a composition including a physiologically active component described herein is administered to a subject to result in targeted expression of a protein or nucleotide sequence that rescues voltage-gated sodium channel function within targeted cell types in the subject. The subject can be an isolated cell, a network of cells, a tissue slice, an experimental animal, a veterinary animal, or a human. In particular embodiments, rescuing voltage-gated sodium channel function includes converting a subject's cells lacking a sufficient quantity and/or activity of Nav1.1 sodium channels, into cells that express a sufficient quantity of exogenous voltage-gated sodium channels and activity, in order to recover neuronal function and to prevent epileptiform circuit activity.

In particular embodiments, rescued voltage-gated sodium channel function is evidenced by one or more of an increase in sodium channel current in and/or the increased excitability of a targeted neuron genetically-modified by the physiologically active component. In particular embodiments, rescued voltage-gated sodium channel function is evidenced by one or more of an increase in sodium channel conductance in and/or the sodium channel influx in response to voltage depolarization of a targeted cell type genetically-modified by the physiologically active component. An increase can be at least a 10% increase, at least a 20% increase, at least a 30% increase, at least a 40% increase, at least a 50% increase, at least a 60% increase, at least a 70% increase, at least an 80% increase or at least a 90% increase. The output of inhibitory neurons can be measured using an electrophysiological method, such as a multi-electrode array or a patch-clamp.

In particular embodiments, the artificial expression constructs are expressed in all neurons. In particular embodiments, the artificial expression constructs are expressed in cell lines.

In particular embodiments, the artificial expression constructs are expressed in a targeted cell type. In particular embodiments, the targeted cell type is a neuron. In particular embodiments, a neuron includes a GABAergic neuron or a glutamatergic neuron. In particular embodiments, the GABAergic neuron is a pan-GABAergic neuron, a forebrain GABAergic neuron, a hippocampal GABAergic neuron, or a cortical GABAergic neuron. In particular embodiments, the glutamatergic neuron includes a forebrain glutamatergic neuron.

In particular embodiments, rescued voltage-gated sodium channel function is evidenced by increased sodium current-dependent fast spiking in forebrain interneurons, for example, using a mouse model. In particular embodiments, rescued voltage-gated sodium channel function is evidenced by delayed or prevented temperature-induced seizing in a mouse model as described herein.

Particular embodiments include identifying a subject with an SCN1A-related seizure disorder in targeted cell types (e.g., forebrain GABAergic neurons). Such subjects can be identified based on a diagnosis of a disorder associated with an SCN1A-related seizure disorder. Such disorders include Dravet syndrome, myoclonic seizures, myoclonic astatic epilepsy (MAE), intractable childhood epilepsy with generalized tonic-clonic seizures, simple febrile seizures, generalized epilepsy and febrile seizures plus (GEFS+), migrating partial seizures of infancy, Lennox-Gastaut syndrome, West syndrome, and seizures.

Regarding Dravet syndrome particularly, 80% of patients with Dravet syndrome test positive for an SCN1A gene mutation, but the absence of an SCN1A mutation does not exclude a Dravet syndrome diagnosis. Dravet syndrome is associated with mutations in SCN1A (such as partial or total deletion mutations, truncating mutations and/or missense mutations e.g., in the voltage or pore regions S4 to S6), SCN1B (encoding the sodium channel β1 subunit), SCN2A, SCN3A, SCN9A, GABRG2 (encoding the γ2 subunit of GABA receptor), GABRD (encoding the delta subunit of GABA receptor) and/or PCDH19 genes.

The methods described herein may be particularly useful for treating children and infants, and for treating disorders that onset during infancy or childhood. In particular embodiments, the patient of the disclosed method is a newborn, a baby, a toddler, a preschooler, a school-age child, or a teenager. In particular embodiments, the patient is 18 years old or younger, 12 years old or younger, 10 years old or younger, 8 years old or younger, 6 years old or younger, 4 years old or younger, 2 years old or younger, 1 year old or younger. In particular embodiments, the patient is an adult that is over eighteen years old.

In particular embodiments, the methods reduce or prevent seizures, or symptoms thereof in a patient in need thereof. In particular embodiments, the methods provided may reduce or prevent one or more different types of seizures. Ideally, the methods of the disclosure result in a total prevention of seizures. However, the disclosure also encompasses methods in which the instances of seizures are decreased by at least 10%, at least 20%, at least 30%, at least 40%, at least 50%, at least 60%, at least 70%, at least 80% or at least 90%.

Generally, a seizure can include convulsions, repetitive movements, unusual sensations, and combinations thereof. Seizures can be categorized as focal seizures (also referred to as partial seizures) and generalized seizures. Focal seizures affect only one side of the brain, while generalized seizures affect both sides of the brain. Specific types of focal seizures include simple focal seizures, complex focal seizures, and secondarily generalized seizures. Simple focal seizures can be restricted or focused on a particular lobe (e.g., temporal lobe, frontal lobe, parietal lobe, or occipital lobe). Complex focal seizures generally affect a larger part of one hemisphere than simple focal seizures, but commonly originate in the temporal lobe or the frontal lobe. When a focal seizure spreads from one side (hemisphere) to both sides of the brain, the seizure is referred to as a secondarily generalized seizure. Specific types of generalized seizures include absences (also referred to as petit mal seizures), tonic seizures, atonic seizures, myoclonic seizures, tonic clonic seizures (also referred to as grand mal seizures), and clonic seizures.

In particular embodiments, methods described herein may reduce the frequency of seizures, reduce the severity of seizures, change the type of seizures (e.g., from a more severe type to a less severe type), or a combination thereof in a patient after treatment compared to the absence of treatment (e.g., before treatment), or compared to treatment with an alternative conventional treatment.

Administration of compositions can be by any appropriate route. For example, in particular embodiments, administration may include administration to a cell or tissue slice for research purposes related to Nav1.1 sodium channel dysfunction. In particular embodiments, administration to a cell or tissue can be done by pipette or injection.

In particular embodiments, administration is to a subject and can be intravenous, retro-orbital, intraocular, intravitreal, parenteral, subcutaneous, intracerebroventricular (ICV), intramuscular, intraparenchymal, intrathecal, intraspinal, intraperitoneal, oral, nasal, or direct to a targeted site administration. Delivery can be accomplished by a needle or a cannula or by any other technique of expelling fluidic materials.

The methods of administration may also include those modalities as described in U.S. Pat. Nos. 5,543,158; 5,641,515 and 5,399,363.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 10⁵ to 10¹⁰⁰ copies of an artificial expression construct of the disclosure. In particular embodiments, a patient receiving intravenous, intraparenchymal, intraspinal, retro-orbital, or intrathecal administration can be infused with from 10⁶ to 10²² copies of the artificial expression construct.

Therapeutically effective amounts include those that provide effective amounts and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model, human study, in vivo, or in vitro assay relevant to a disorder associated with Nav1.1 sodium channel dysfunction.

A “therapeutic treatment” includes a treatment administered to a subject who displays symptoms or signs of a disorder associated with Nav1.1 sodium channel dysfunction and is administered to the subject for the purpose of diminishing or eliminating those signs or symptoms of the disorder. The therapeutic treatment can reduce, control, or eliminate the presence or activity of the disorder, the cause of the disorder, and/or reduce control or eliminate side effects of the disorder.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of expression constructs to provide therapeutic benefit to the subject receiving the administration.

Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions.

For example, the number of expression constructs administered to a subject may be 10⁷, 10⁸, 10⁹, 10¹⁰, 10¹¹, 10¹², 10¹³, or even higher, expression constructs/ml given either as a single dose, or divided into two or more administrations as may be required to achieve a desired physiological outcome. In particular embodiments, it may be desirable to administer two or more different expression constructs, either alone, or in combination with one or more other therapeutic drugs to achieve the desired effects of a particular therapy regimen. In particular embodiments, in certain embodiments, it may be desirable to administer two or more different expression constructs in combination to achieve a desired effect.

Particular dosing and timing of administration for a particular subject can be chosen by a treating physician, researcher, or veterinarian. In other words, the amount of compositions and/or expression constructs and time of administration will be within the purview of the skilled artisan having benefit of the present teachings.

In particular embodiments, treatments for Nav1.1 sodium channel disorders can be combined with another treatment. For example, common conventional therapies for seizures and epilepsy include antiepileptic drugs and non-antiepileptic drug treatments such as low carbohydrate diet (e.g., ketogenic diets, such as classical diet, medium chain triglyceride (MCT) diet, modified Atkins diet (MAD), and low glycemic index treatment (LGIT)), intravenous immunoglobulin, steroids, elimination diet, vagus nerve stimulation, corticetomy, and multiple subpial transections.

Common antiepileptic and anticonvulsive active compounds that may be used in combination with compositions described herein include acetazolamide, cannabidiol, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

In particular embodiments, a method of rescuing voltage-gated sodium channel function within a targeted population of cells includes co-administering a therapeutically effective amount of a first artificial expression construct and a therapeutically effective amount of a second artificial expression construct as described elsewhere herein, to a sample or subject including the targeted population of cells thereby rescuing voltage-gated sodium channel function within a targeted population of cells. The term “co-administering” refers to administering different constructs in a manner such that their expression products interact to form functional SCN1A within a cell. The co-administration can be simultaneous administration or can include sequential administration.

(vi) Kits and Commercial Packages. Kits and commercial packages contain an artificial expression construct described herein. The artificial expression construct can be isolated. In particular embodiments, the components of an expression product can be isolated from each other. In particular embodiments, the expression product can be within a vector, within a viral vector, within a cell, within a tissue slice or sample, and/or within a transgenic animal. In particular embodiments, an animal is transgenic following administration of a composition including the expression construct. In particular embodiments, a transgenic animal includes a genetic modification that renders the animal appropriate for use in an animal model of Dravet syndrome. For example, the transgenic animal such as a mouse can be Scn1a^{+/-}.

Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition (e.g., Nav1.1 sodium channel dysfunction, such as epilepsy and/or Dravet syndrome).

The Exemplary Embodiments and Experimental Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (VII) EXEMPLARY EMBODIMENTS

1. A system to express an SCN1A coding sequence in a subject in need thereof including
(i) a first artificial expression construct including a first portion of the SCN1A coding sequence, an N-intein coding sequence, and a first promoter sequence, wherein the N-intein coding sequence is located at the 3′ end of the first portion of the SCN1A coding sequence; and
(ii) a second artificial expression construct including a second portion of the SCN1A coding sequence, a C-intein coding sequence, and a second promoter sequence, wherein the C-intein coding sequence is located at the 5′ end of the second portion of the SCN1A coding sequence;
wherein when the first artificial expression construct and the second artificial expression construct are expressed in a central nervous cell type, the protein product of the first portion of the SCN1A coding sequence and the protein of product of the second portion SCN1A coding sequence are linked to form a functional SCN1A protein within the central nervous cell type.

2. The system of embodiment 1, wherein the first portion of the SCN1A coding sequence includes a sequence having at least 90% sequence identity to SEQ ID NO: 59, SEQ ID NO: 61, or SEQ ID NO: 63.

3. The system of embodiments 1 or 2, wherein the first portion of the SCN1A coding sequence includes hSCN1A-CO-Nterm1049 (SEQ ID NO: 59), hSCN1A-CO-Nterm956 (SEQ ID NO: 61), or hSCN1A-CO-Nterm947 (SEQ ID NO: 63).

4. The system of any of embodiments 1-3, wherein the second portion of the SCN1A coding sequence includes a sequence having at least 90% sequence identity to SEQ ID NO: 60, SEQ ID NO: 62, or SEQ ID NO: 64.

5. The system of any of embodiments 1-4, wherein the second portion of the SCN1A coding sequence includes hSCN1A-CO-Cterm949 (SEQ ID NO: 60), hSCN1A-CO-Cterm1042 (SEQ ID NO: 62), or hSCN1A-CO-Cterm1051 (SEQ ID NO: 64).

6. The system of any of embodiments 1-5, wherein the protein product of the N-intein coding sequence and the protein product of the C-intein coding sequence include a split intein after protein splicing.

7. The system of any of embodiments 1-6, wherein the N-intein coding sequence includes a Cfa-N coding sequence.

8. The system of embodiment 7, wherein the Cfa-N coding sequence includes the sequence as set forth in SEQ ID NO: 57 or a sequence having at least 90% sequence identity to SEQ ID NO: 57.

9. The system of any of embodiments 1-8, wherein the C-intein coding sequence includes a Cfa-C coding sequence.

10. The system of embodiment 9, wherein the Cfa-C coding sequence includes the sequence as set forth in SEQ ID NO: 58 or a sequence having at least 90% sequence identity to SEQ ID NO: 58.

11. The system of any of embodiments 1-10, wherein the first promoter includes a minBglobin promoter, an hSyn1 promoter, a shortened hSyn1 promoter, or a CMV promoter.

12. The system of embodiment 11, wherein the minBglobin promoter includes the sequence as set forth in SEQ ID NO: 3 or a sequence having at least 90% sequence identity to SEQ ID NO: 3.

13. The system of embodiment 11, wherein the hSyn1 promoter includes the sequence as set forth in SEQ ID NO: 52 or a sequence having at least 90% sequence identity to SEQ ID NO: 52.

14. The system of embodiment 11, wherein the shortened hSyn1 promoter includes the sequence as set forth in SEQ ID NO: 54 or a sequence having at least 90% sequence identity to SEQ ID NO: 54.

15. The system of embodiment 11, wherein the CMV promoter includes the sequence as set forth in SEQ ID NO: 4 or a sequence having at least 90% sequence identity to SEQ ID NO: 4

16. The system of any of embodiments 1-15, wherein the second promoter includes a minBglobin promoter, an hSyn1 promoter, a shortened hSyn1 promoter, or a CMV promoter.

17. The system of embodiment 16, wherein the minBglobin promoter includes the sequence as set forth in SEQ ID NO: 3 or a sequence having at least 90% sequence identity to SEQ ID NO: 3.

18. The system of embodiment 16, wherein the hSyn1 promoter includes the sequence as set forth in SEQ ID NO: 52 or a sequence having at least 90% sequence identity to SEQ ID NO: 52.

19. The system of embodiment 16, wherein the shortened hSyn1 promoter includes the sequence as set forth in SEQ ID NO: 54 or a sequence having at least 90% sequence identity to SEQ ID NO: 54.

20. The system of embodiment 16, wherein the CMV promoter includes the sequence as set forth in SEQ ID NO: 4 or a sequence having at least 90% sequence identity to SEQ ID NO: 4.

21. The system of any of embodiments 1-20, wherein the first artificial expression construct further includes a first enhancer sequence that leads to targeted expression of the first portion of the SCN1A coding sequence within a targeted central nervous system cell type.

22. The system of any of embodiments 1-21, wherein the second artificial expression construct further includes a second enhancer sequence that leads to targeted expression of the second portion of the SCN1A coding sequence within a targeted central nervous system cell type.

23. The system of any of embodiments 1-22, wherein the first artificial expression construct further comprises a first enhancer sequence that leads to targeted expression of the first portion of the SCN1A coding sequence within a targeted central nervous system cell type and the second artificial expression construct further comprises a second enhancer sequence that leads to targeted expression of the second portion of the SCN1A coding sequence within the targeted central nervous system cell type

24. The system of any of embodiments 1-23, wherein the targeted central nervous cell type includes a neuron.

25. The system of embodiment 24, wherein the neuron includes a GABAergic neuron or a glutamatergic neuron.

26. The system of embodiment 25, wherein the GABAergic neuron includes a forebrain GABAergic neuron.

27. The system of embodiment 25 or 26, wherein the GABAergic neuron includes a pan-GABAergic neuron.

28. The system of embodiment any of embodiments 25-27 wherein the glutamatergic neuron includes a forebrain glutamatergic neuron.

29. The system of any of embodiments 1-28, wherein the targeted central nervous cell type includes a cell line.

30. The system of any of embodiments 23-29, wherein the first enhancer sequence includes DLX2.0 (SEQ ID NO: 2), the second enhancer sequence includes DLX2.0 (SEQ ID NO: 2), and the targeted central nervous cell type is a forebrain GABAergic neuron.

31. The system of any of embodiments 23-29, wherein the first enhancer sequence includes eHGT_078h (SEQ ID NO: 55), the second enhancer sequence includes eHGT_078h (SEQ ID NO: 55), and the targeted central nervous cell type is a forebrain glutamatergic neuron.

32. The system of any of embodiments 1-31, wherein the first artificial expression construct further includes a first miRNA binding site that leads to targeted expression of the first portion of the SCN1A coding sequence within a targeted central nervous system cell type.

33. The system of any of embodiments 1-32, wherein the second artificial expression construct further includes a second miRNA binding site that leads to targeted expression of the second portion of the SCN1A coding sequence within the targeted central nervous system cell type.

34. The system of any of embodiments 1-33, wherein the first artificial expression construct further includes a first miRNA binding site that leads to targeted expression of the first portion of the SCN1A coding sequence within a targeted central nervous system cell type and the second artificial expression construct further includes a second miRNA binding site that leads to targeted expression of the second portion of the SCN1A coding sequence within the targeted central nervous system cell type.

35. The system of embodiment 34, wherein the first miRNA binding site includes 4×2C miRNA binding site (SEQ ID NO: 56), the second miRNA binding site includes 4×2C miRNA binding site (SEQ ID NO: 56), and the targeted central nervous system cell type includes a pan-GABAergic neuron.

36. The system of any of embodiments 1-35, wherein the first and second artificial expression constructs, respectively, include or encode a set of features selected from:
DLX2.0-minBG-2×HA-hSCN1A-CO-Nterm1049-Intron-CfaN-WPRE3-SV40 pA and
DLX2.0-minBG-CfaC-hSCN1A-CO-Cterm949-Intron-3×FLAG-WPRE3-SV40 pA;
hSyn1-2×HA-hSCN1A-CO-Nterm1049-Intron-CfaN-WPRE3-shortPolyA and
hSyn1-CfaC-hSCN1A-CO-Cterm949-Intron-3×FLAG-WPRE3-shortPolyA;
CMV-2×HA-hSCN1A-CO-Nterm1049-Intron-CfaN_IRES2_SYFP2_BGHpA and
CMV-CfaC-hSCN1A-CO-Cterm949-Intron-3×FLAG_IRES2_mScarlet-3×NLS_BGHpA;
hSyn1short-2×HA-hSCN1A-CO-Nterm1049-Intron-CfaN-4×2C-WPRE3-shortPolyA and
hSyn1short-CfaC-hSCN1A-CO-Cterm949-Intron-3×FLAG-4×2C-WPRE3-shortPolyA;
eHGT_078 h-minBG-2×HA-hSCN1A-CO-Nterm1049-Intron-CfaN-WPRE3-SV40 pA and
eHGT_078 h-minBG-CfaC-hSCN1A-CO-Cterm949-Intron-3×FLAG-WPRE3-SV40 pA;
hSyn1-2×HA-hSCN1A-CO-Nterm956-Intron-CfaN-WPRE3-shortPolyA and
hSyn1-CfaC-hSCN1A-CO-Cterm1042-Intron-3×FLAG-WPRE3-shortPolyA;
hSyn1-2×HA-hSCN1A-CO-Nterm947-Intron-CfaN-WPRE3-shortPolyA and
hSyn1-CfaC-hSCN1A-CO-Cterm1051-Intron-3×FLAG-WPRE3-shortPolyA;
DLX2.0-minBG-hSCN1A-CO-Nterm1049-Intron-CfaN-[post regulatory element] and
DLX2.0-minBG-CfaC-hSCN1A-CO-Cterm949-Intron-[post regulatory element];
hSyn1-hSCN1A-CO-Nterm1049-Intron-CfaN-[post regulatory element] and
hSyn1-CfaC-hSCN1A-CO-Cterm949-Intron-[post regulatory element];
CMV-hSCN1A-CO-Nterm1049-Intron-CfaN-[post regulatory element] and
CMV-CfaC-hSCN1A-CO-Cterm949-Intron-[post regulatory element];
hSyn1short-hSCN1A-CO-Nterm1049-Intron-CfaN-4×2C-[post regulatory element] and
hSyn1short-CfaC-hSCN1A-CO-Cterm949-Intron-4×2C-[post regulatory element];
eHGT_078 h-minBG-hSCN1A-CO-Nterm1049-Intron-CfaN-[post regulatory element] and
eHGT_078 h-minBG-CfaC-hSCN1A-CO-Cterm949-Intron-[post regulatory element];
hSyn1-hSCN1A-CO-Nterm956-Intron-CfaN-[post regulatory element] and
hSyn1-CfaC-hSCN1A-CO-Cterm1042-Intron-[post regulatory element]; or
hSyn1-hSCN1A-CO-Nterm947-Intron-CfaN-[post regulatory element] and
hSyn1-CfaC-hSCN1A-CO-Cterm1051-Intron-[post regulatory element].

37. An artificial expression construct including a portion of an SCN1A coding sequence, an N-intein coding sequence, and a promoter sequence, wherein the N-intein coding sequence is located at the 3′ end of the first portion of the SCN1A coding sequence.

38. The artificial expression construct of embodiment 37, wherein the portion of the SCN1A coding sequence includes a sequence having at least 90% sequence identity to SEQ ID NO: 59, SEQ ID NO: 61, or SEQ ID NO: 63.

39. The artificial expression construct of embodiments 37 or 38, wherein the portion of the SCN1A coding sequence includes hSCN1A-CO-Nterm1049 (SEQ ID NO: 59), hSCN1A-CO-Nterm956 (SEQ ID NO: 61), or hSCN1A-CO-Nterm947 (SEQ ID NO: 63).

40. The artificial expression construct of any of embodiments 37-39, wherein the N-intein coding sequence includes a Cfa-N coding sequence.

41. The artificial expression construct of embodiment 40, wherein the Cfa-N coding sequence includes the sequence as set forth in SEQ ID NO: 57 or a sequence having at least 90% sequence identity to SEQ ID NO: 57.

42. The artificial expression construct of any of embodiments 37-41, wherein the promoter sequence includes a minBglobin promoter, an hSyn1 promoter, a CMV promoter, or a shortened hSyn1 promoter.

43. The artificial expression construct of any of embodiments 37-42, further including an enhancer sequence that leads to targeted expression of the portion of the SCN1A coding sequence within a targeted central nervous system cell type.

44. The artificial expression construct of embodiment 43, wherein the enhancer sequence includes DLX2.0 (SEQ ID NO: 2) and the targeted central nervous system cell type includes a forebrain GABAergic neuron.

45. The artificial expression construct of embodiment 43, wherein the enhancer sequence includes eHGT_078 h and the targeted central nervous system cell type includes a forebrain glutamatergic neuron.

46. The artificial expression construct of any of embodiments 37-45, further including an miRNA binding site that leads to targeted expression of the portion of the SCN1A coding sequence within a targeted central nervous system cell type.

47. The artificial expression construct of embodiment 46, wherein the miRNA binding site includes a 4×2c miRNA binding site and the targeted central nervous cell type includes a pan-GABAergic neuron.

48. The artificial expression construct of any of embodiments 37-47, wherein the artificial expression construct further encodes a reporter protein.

49. The artificial expression construct of embodiment 48, wherein the reporter protein includes a fluorescent protein or a tag cassette.

50. The artificial expression construct of embodiment 49, wherein the tag cassette includes a sequence as set forth in SEQ ID NOs: 15 and 34-45.

51. The artificial expression construct of any of embodiments 37-50, wherein the artificial expression construct is within an adeno-associated viral (AAV) vector.

52. The artificial expression construct of any of embodiments 37-51, wherein the artificial expression construct is associated with a capsid that crosses the blood brain barrier.

53. The artificial expression construct of embodiment 52, wherein the capsid includes PHP.eB, AAV-BR1, AAV-PHP.S, AAV-PHP.B, or AAV-PPS.

54. The artificial expression construct of any of embodiments 37-53, wherein the artificial expression construct includes or encodes a skipping element.

55. The artificial expression construct of embodiment 54, wherein the skipping element includes a 2A peptide and/or an internal ribosome entry site (IRES).

56. The artificial expression construct of embodiment 55, wherein the IRES includes IRES2.

57. The artificial expression construct of embodiment 55, wherein the 2A peptide includes T2A, P2A, E2A, or F2A.

58. The artificial expression construct of any of embodiments 37-57, wherein the artificial expression construct includes or encodes a set of features selected from: DLX2.0 (SEQ ID NO: 2), 4×2C miR binding site (SEQ ID NO: 56), 8×2C miR binding site (SEQ ID NO: 87), eHGT_078h, inteins, AAV, scAAV, rAAV, pAAV, pSMART-HCKan, minBglobin, CMV promoter, hSyn1 promoter, shortened hSyn1 promoter, minCMV, minCMV*, minRho, minRho*, fluorescent protein (e.g., EGFP, SYFP, GFP), hsA2, Cre, iCre, dgCre, FlpO, tTA2, SP10 (e.g., 3×SP10), tag cassette, intron, 10aa, nuclear localization protein, self-cleaving peptides, WPRE, WPRE3, hGHpA, SV40 pA, shortPolyA, and/or BGHpA.

59. An artificial expression construct including a portion of an SCN1A coding sequence, a C-intein coding sequence, an enhancer sequence that leads to targeted expression of the portion of the SCN1A coding sequence within a targeted central nervous system cell type, and a promoter sequence, wherein the C-intein coding sequence is located at the 5′ end of the portion of the SCN1A coding sequence.

60. The artificial expression construct of embodiment 59, wherein the second portion of the SCN1A coding sequence includes a sequence having at least 90% sequence identity to SEQ ID NO: 60, SEQ ID NO: 62, or SEQ ID NO: 64.

61. The artificial expression construct of embodiment 59 or 60, wherein the second portion of the SCN1A coding sequence includes hSCN1A-CO-Cterm949 (SEQ ID NO: 60), hSCN1A-CO-Cterm1042, (SEQ ID NO: 62) or hSCN1A-CO-Cterm1051 (SEQ ID NO: 64).

62. The artificial expression construct of any of embodiments 59-61, wherein the C-intein coding sequence includes a Cfa-C coding sequence.

63. The artificial expression construct of embodiment 62, wherein the Cfa-C coding sequence includes the sequence as set forth in SEQ ID NO: 58 or a sequence having at least 90% sequence identity to SEQ ID NO: 58.

64. The artificial expression construct of any of embodiments 59-63, further including an enhancer sequence that leads to targeted expression of the portion of the SCN1A coding sequence within a targeted central nervous system cell type.

65. The artificial expression construct of embodiment 64, wherein the enhancer sequence includes DLX2.0 (SEQ ID NO: 2) and the targeted central nervous system cell type includes a forebrain GABAergic neuron.

66. The artificial expression construct of embodiment 64, wherein the enhancer sequence includes eHGT_078 h and the targeted central nervous system cell type includes a forebrain glutamatergic neuron.

67. The artificial expression construct of any of embodiments 59-66, further including a miRNA binding site that leads to targeted expression of the portion of the SCN1A coding sequence within a targeted central nervous system cell type.

68. The artificial expression construct of embodiment 67, wherein the miRNA binding site includes a 4×2c miRNA binding site and the targeted central nervous cell type includes a pan-GABAergic neuron.

69. The artificial expression construct of any of embodiments 59-68, wherein the artificial expression construct further encodes a reporter protein.

70. The artificial expression construct of embodiment 69, wherein the reporter protein includes a fluorescent protein or a tag cassette.

71. The artificial expression construct of embodiment 70, wherein the tag cassette includes a sequence as set forth in SEQ ID NOs: 15 and 34-45.

72. The artificial expression construct of any of embodiments 59-71, wherein the artificial expression construct is within an adeno-associated viral (AAV) vector.

73. The artificial expression construct of any of embodiments 59-72, wherein the artificial expression construct is associated with a capsid that crosses the blood brain barrier.

74. The artificial expression construct of embodiment 73, wherein the capsid includes PHP.eB, AAV-BR1, AAV-PHP.S, AAV-PHP.B, or AAV-PPS.

75. The artificial expression construct of any of embodiments 59-74, wherein the artificial expression construct includes or encodes a skipping element.

76. The artificial expression construct of embodiment 75, wherein the skipping element includes a 2A peptide and/or an internal ribosome entry site (IRES).

77. The artificial expression construct of embodiment 76, wherein the IRES includes IRES2.

78. The artificial expression construct of embodiment 76 or 77, wherein the 2A peptide includes T2A, P2A, E2A, or F2A.

79. The artificial expression construct of any of embodiments 59-78, wherein the artificial expression construct includes or encodes a set of features selected from: DLX2.0 (SEQ ID NO: 2), 4×2C miR binding site (SEQ ID NO: 56), 8×2C miR binding site (SEQ ID NO: 87), eHGT_078h, inteins, AAV, scAAV, rAAV, pAAV, pSMART-HCKan, minBglobin, CMV promoter, hSyn1 promoter, shortened hSyn1 promoter, minCMV, minCMV*, minRho, minRho*, fluorescent protein (e.g., EGFP, SYFP, GFP), hsA2, Cre, iCre, dgCre, FlpO, tTA2, SP10 (e.g., 3×SP10), tag cassette, intron, 10aa, nuclear localization protein, self-cleaving peptides, WPRE, WPRE3, hGHpA, SV40 pA, shortPolyA, and/or BGHpA.

80. An administrable composition including the system of any of embodiments 1-36, the artificial expression construct of any of embodiments 37-58, and/or the artificial expression construct of any of embodiments 59-79.

81. A vector including the artificial expression construct of any of embodiments 37-58 or the artificial expression construct of any of embodiments 59-79.

82. The vector of embodiment 81, wherein the vector includes a viral vector.

83. A vector system including the system of any of embodiments 1-36, wherein the first artificial expression construct is within a first vector and the second artificial expression construct is within a second vector.

84. The vector system of embodiment 83, wherein the first vector includes the elements of CN3252, CN3683, CN3251, CN3677, CN4541, CN4217, or CN4642.

85. The vector system of embodiments 83 or 84, wherein the second vector includes the elements of CN3254, CN3684, CN3253, CN3678, CN4542, CN4218, or CN4643.

86. A transgenic cell including the system of any of embodiments 1-46.

87. The transgenic cell of embodiment 86, wherein the transgenic cell includes a neuron.

88. The transgenic cell of embodiments 86 or 87, wherein the transgenic cell is a GABAergic neuron or a glutamatergic neuron.

89. The transgenic cell of embodiment 88, wherein the GABAergic neuron is a pan-GABAergic neurons, forebrain GABAergic neurons, hippocampal GABAergic neurons, and/or cortical GABAergic neurons.

90. The transgenic cell of embodiments 88 or 89, wherein the GABAergic neuron is a forebrain GABAergic neuron.

91. The transgenic cell of embodiments 88 or 89, wherein the glutamatergic neuron includes a forebrain glutamatergic neuron.

92. The transgenic cell of any of embodiments 86-91, wherein the transgenic cell includes a cell line.

93. The transgenic cell of any of embodiments 86-92, wherein the transgenic cell is murine, human, or non-human primate.

94. A non-human transgenic animal including the system of any of embodiments 1-36 and/or the transgenic cell of any of embodiments 86-93.

95. The non-human transgenic animal of embodiment 94, wherein the non-human transgenic animal is a mouse or a non-human primate.

96. A kit including the system of any of embodiments 1-36, the artificial expression construct of any of embodiments 37-58 or the artificial expression construct of any of embodiments 59-79, the transgenic cell of any of embodiments 86-93, and/or the non-human transgenic animal of embodiments 94 or 95.

97. A method of rescuing voltage-gated sodium channel function within a targeted population of cells, the method including co-administering a therapeutically effective amount of the artificial expression construct of any of embodiments 37-58 and a therapeutically effective amount of the artificial expression construct of any of embodiments 59-79, to a sample or subject including the targeted population of cells thereby rescuing voltage-gated sodium channel function within a targeted population of cells.

98. The method of embodiment 97, wherein the co-administering includes pipetting.

99. The method of embodiment 98, wherein the pipetting is to a brain slice.

100. The method of embodiment 99, wherein the brain slice includes a neuron.

101. The method of embodiments 99 or 100, wherein the brain slice includes a GABAergic neuron or a glutamatergic neurons.

102. The method of embodiment 101, wherein the GABAergic neuron includes a pan-GABAergic neuron, a forebrain GABAergic neuron, a hippocampal GABAergic neuron, or a cortical GABAergic neuron.

103. The method of embodiments 101 or 102, wherein the glutamatergic neuron includes a forebrain glutamatergic neuron.

104. The method of any of embodiments 99-103, wherein the brain slice includes a cell line.

105. The method of any of embodiments 99-104, wherein the brain slice is murine, human, or non-human primate.

106. The method of any of embodiments 97-105, wherein the co-administering includes administering to a living subject.

107. The method of embodiment 106, wherein the living subject is a human, non-human primate, or a mouse.

108. The method of embodiments 106 or 107, wherein the living subject is in need thereof due to a diagnosis of an SCN1A-related seizure disorder.

109. The method of embodiment 108, wherein the SCN1A-related seizure disorder includes Dravet syndrome, myoclonic seizures, myoclonic astatic epilepsy (MAE), intractable childhood epilepsy with generalized tonic-clonic seizures, simple febrile seizures, generalized epilepsy and febrile seizures plus (GEFS+), migrating partial seizures of infancy, Lennox-Gastaut syndrome, or West syndrome.

110. The method of any of embodiments 106-109, wherein the living subject is a pediatric patient.

111. The method of any of embodiments 106-110, wherein the living subject is less than 4 years old.

112. The method of any of embodiments 106-109, wherein the living subject is a transgenic non-human animal.

113. The method of any of embodiments 97-112, wherein the co-administering includes intravenous injection, intraparenchymal injection into brain tissue, intracerebroventricular (ICV) injection, intra-cisterna magna (ICM) injection, or intrathecal injection.

114. An artificial expression construct including CN3252, CN3254, CN3683, CN3684, CN3251, CN3253, CN3677, CN3678, CN4541, CN4542, CN4217, CN4218, CN4642, or CN4643.

(viii) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, Ile, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term concatenate is broadly used to describe linking together into a chain or series. It is used to describe the linking together of nucleotide or amino acid sequences into a single nucleotide or amino acid sequence, respectively. The term “concatamerize” should be interpreted to recite: “concatenate.”

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in targeted expression of SCN1A in the targeted cell type utilizing an artificial expression construct disclosed herein.

In particular embodiments, artificial means not naturally occurring.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In dosing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A system to express a functional SCN1A protein in a subject in need thereof comprising
(i) a first artificial expression construct comprising
a first portion of an SCN1A coding sequence, the first portion having the sequence as set forth in SEQ ID NO: 59;
an N-intein coding sequence as set forth in SEQ ID NO: 57; and
a first promoter sequence;
wherein the N-intein coding sequence is located at the 3′ end of the first portion of the SCN1A coding sequence; and
(ii) a second artificial expression construct comprising
a second portion of the SCN1A coding sequence, the second portion having the sequence as set forth in SEQ ID NO: 60;
a C-intein coding sequence as set forth in SEQ ID NO: 58; and
a second promoter sequence;
wherein the C-intein coding sequence is located at the 5′ end of the second portion of the SCN1A coding sequence;
wherein when the first artificial expression construct and the second artificial expression construct are expressed in a central nervous cell type, the protein product of the first portion of the SCN1A coding sequence and the protein product of the second portion of the SCN1A coding sequence are linked to form the functional SCN1A protein within the central nervous cell type.

2. A system to express a functional SCN1A protein in a subject in need thereof comprising
(i) a first artificial expression construct comprising a first portion of an SCN1A coding sequence, an N-intein coding sequence, and a first promoter sequence, wherein the N-intein coding sequence is located at the 3′ end of the first portion of the SCN1A coding sequence; and
(ii) a second artificial expression construct comprising a second portion of the SCN1A coding sequence, a C-intein coding sequence, and a second promoter sequence, wherein the C-intein coding sequence is located at the 5′ end of the second portion of the SCN1A coding sequence;
wherein when the first artificial expression construct and the second artificial expression construct are expressed in a central nervous cell type, the protein product of the first portion of the SCN1A coding sequence and the protein product of the second portion of the SCN1A coding sequence are linked to form the functional SCN1A protein within the central nervous cell type.

3. The system of claim 2, wherein the first portion of the SCN1A coding sequence comprises a sequence having at least 90% sequence identity to SEQ ID NO: 59, SEQ ID NO: 61, or SEQ ID NO: 63.

4. The system of claim 2, wherein the first portion of the SCN1A coding sequence comprises hSCN1A-CO-Nterm1049 (SEQ ID NO: 59), hSCN1A-CO-Nterm956 (SEQ ID NO: 61), or hSCN1A-CO-Nterm947 (SEQ ID NO: 63).

5. The system of claim 2, wherein the second portion of the SCN1A coding sequence comprises a sequence having at least 90% sequence identity to SEQ ID NO: 60, SEQ ID NO: 62, or SEQ ID NO: 64.

6. The system of claim 2, wherein the second portion of the SCN1A coding sequence comprises hSCN1A-CO-Cterm949 (SEQ ID NO: 60), hSCN1A-CO-Cterm1042 (SEQ ID NO: 62), or hSCN1A-CO-Cterm1051 (SEQ ID NO: 64).

7. The system of claim 2, wherein the protein product of the N-intein coding sequence and the protein product of the C-intein coding sequence comprise a split intein after protein splicing.

8. The system of claim 2, wherein the N-intein coding sequence comprises a Cfa-N coding sequence.

9. The system of claim 8, wherein the Cfa-N coding sequence comprises the sequence as set forth in SEQ ID NO: 57 or a sequence having at least 90% sequence identity to SEQ ID NO: 57.

10. The system of claim 2, wherein the C-intein coding sequence comprises a Cfa-C coding sequence.

11. The system of claim 10, wherein the Cfa-C coding sequence comprises the sequence as set forth in SEQ ID NO: 58 or a sequence having at least 90% sequence identity to SEQ ID NO: 58.

12. The system of claim 2, wherein the first promoter comprises a minBglobin promoter, an hSyn1 promoter, a shortened hSyn1 promoter, or a CMV promoter.

13. The system of claim 12, wherein the minBglobin promoter comprises the sequence as set forth in SEQ ID NO: 3 or a sequence having at least 90% sequence identity to SEQ ID NO: 3.

14. The system of claim 12, wherein the hSyn1 promoter comprises the sequence as set forth in SEQ ID NO: 52 or a sequence having at least 90% sequence identity to SEQ ID NO: 52.

15. The system of claim 12, wherein the shortened hSyn1 promoter comprises the sequence as set forth in SEQ ID NO: 54 or a sequence having at least 90% sequence identity to SEQ ID NO: 54.

16. The system of claim 12, wherein the CMV promoter comprises the sequence as set forth in SEQ ID NO: 4 or a sequence having at least 90% sequence identity to SEQ ID NO: 4.

17. The system of claim 2, wherein the second promoter comprises a minBglobin promoter, an hSyn1 promoter, a shortened hSyn1 promoter, or a CMV promoter.

18. The system of claim 12, wherein the minBglobin promoter comprises the sequence as set forth in SEQ ID NO: 3 or a sequence having at least 90% sequence identity to SEQ ID NO: 3.

19. The system of claim 12, wherein the hSyn1 promoter comprises the sequence as set forth in SEQ ID NO: 52 or a sequence having at least 90% sequence identity to SEQ ID NO: 52.

20. The system of claim 14, wherein the shortened hSyn1 promoter comprises the sequence as set forth in SEQ ID NO: 54 or a sequence having at least 90% sequence identity to SEQ ID NO: 54.

21. The system of claim 12, wherein the CMV promoter comprises the sequence as set forth in SEQ ID NO: 4 or a sequence having at least 90% sequence identity to SEQ ID NO: 4.

22. The system of claim 2, wherein the first artificial expression construct further comprises a first enhancer sequence that leads to targeted expression of the first portion of the SCN1A coding sequence within a targeted central nervous system cell type.

23. The system of claim 2, wherein the second artificial expression construct further comprises a second enhancer sequence that leads to targeted expression of the second portion of the SCN1A coding sequence within a targeted central nervous system cell type.

24. The system of claim 2, wherein the first artificial expression construct further comprises a first enhancer sequence that leads to targeted expression of the first portion of the SCN1A coding sequence within a targeted central nervous system cell type and the second artificial expression construct further comprises a second enhancer sequence that leads to targeted expression of the second portion of the SCN1A coding sequence within the targeted central nervous system cell type.

25. The system of claim 2, wherein the targeted central nervous cell type comprises a neuron.

26. The system of claim 25, wherein the neuron comprises a GABAergic neuron or a glutamatergic neuron.

27. The system of claim 26, wherein the GABAergic neuron comprises a forebrain GABAergic neuron.

28. The system of claim 26, wherein the GABAergic neuron comprises a pan-GABAergic neuron.

29. The system of claim 26, wherein the glutamatergic neuron comprises a forebrain glutamatergic neuron.

30. The system of claim 2, wherein the targeted central nervous cell type comprises a cell line.

31. The system of claim 24, wherein the first enhancer sequence comprises DLX2.0 (SEQ ID NO: 2), the second enhancer sequence comprises DLX2.0 (SEQ ID NO: 2), and the targeted central nervous cell type is a forebrain GABAergic neuron.

32. The system of claim 24, wherein the first enhancer sequence comprises eHGT_078h (SEQ ID NO: 55), the second enhancer sequence comprises eHGT_078h (SEQ ID NO: 55), and the targeted central nervous cell type is a forebrain glutamatergic neuron.

33. The system of claim 2, wherein the first artificial expression construct further comprises a first miRNA binding site that leads to targeted expression of the first portion of the SCN1A coding sequence within a targeted central nervous system cell type.

34. The system of claim 2, wherein the second artificial expression construct further comprises a second miRNA binding site that leads to targeted expression of the second portion of the SCN1A coding sequence within a targeted central nervous system cell type.

35. The system of claim 2, wherein the first artificial expression construct further comprises a first miRNA binding site that leads to targeted expression of the first portion of the SCN1A coding sequence within a targeted central nervous system cell type and the second artificial expression construct further comprises a second miRNA binding site that leads to targeted expression of the second portion of the SCN1A coding sequence within the targeted central nervous system cell type.

36. The system of claim 35, wherein the first miRNA binding site comprises 4×2C miRNA binding site (SEQ ID NO: 56), the second miRNA binding site comprises 4×2C miRNA binding site (SEQ ID NO: 56), and the targeted central nervous system cell type comprises a pan-GABAergic neuron.

37. The system of claim 2, wherein the first and second artificial expression constructs, respectively, comprise or encode a set of features selected from:
DLX2.0-minBglobin-2×HA-hSCN1A-CO-Nterm1049-Intron-CfaN-WPRE3-SV40 pA and
DLX2.0-minBglobin-CfaC-hSCN1A-CO-Cterm949-Intron-3×FLAG-WPRE3-SV40 pA;
hSyn1-2×HA-hSCN1A-CO-Nterm1049-Intron-CfaN-WPRE3-shortPolyA and
hSyn1-CfaC-hSCN1A-CO-Cterm949-Intron-3×FLAG-WPRE3-shortPolyA;
CMVpromoter-2×HA-hSCN1A-CO-Nterm1049-Intron-CfaN_IRES2_SYFP2_BGHpA and
CMVpromoter-CfaC-hSCN1A-CO-Cterm949-Intron-3×FLAG_IRES2_mScarlet-3×NLS_BGHpA;
hSyn1short-2×HA-hSCN1A-CO-Nterm1049-Intron-CfaN-4×2C-WPRE3-shortPolyA and
hSyn1short-CfaC-hSCN1A-CO-Cterm949-Intron-3×FLAG-4×2C-WPRE3-shortPolyA;
eHGT_078 h-minBglobin-2×HA-hSCN1A-CO-Nterm1049-Intron-CfaN-WPRE3-SV40 pA and
eHGT_078 h-minBglobin-CfaC-hSCN1A-CO-Cterm949-Intron-3×FLAG-WPRE3-SV40 pA;
hSyn1-2×HA-hSCN1A-CO-Nterm956-Intron-CfaN-WPRE3-shortPolyA and
hSyn1-CfaC-hSCN1A-CO-Cterm1042-Intron-3×FLAG-WPRE3-shortPolyA;
hSyn1-2×HA-hSCN1A-CO-Nterm947-Intron-CfaN-WPRE3-shortPolyA and
hSyn1-CfaC-hSCN1A-CO-Cterm1051-Intron-3×FLAG-WPRE3-shortPolyA;
DLX2.0-minBglobin-hSCN1A-CO-Nterm1049-Intron-CfaN-[post regulatory element] and
DLX2.0-minBglobin-CfaC-hSCN1A-CO-Cterm949-Intron-[post regulatory element];
hSyn1-hSCN1A-CO-Nterm1049-Intron-CfaN-[post regulatory element] and
hSyn1-CfaC-hSCN1A-CO-Cterm949-Intron-[post regulatory element];
CMVpromoter-hSCN1A-CO-Nterm1049-Intron-CfaN-[post regulatory element] and
CMVpromoter-CfaC-hSCN1A-CO-Cterm949-Intron-[post regulatory element];
hSyn1short-hSCN1A-CO-Nterm1049-Intron-CfaN-4×2C-[post regulatory element] and
hSyn1short-CfaC-hSCN1A-CO-Cterm949-Intron-4×2C-[post regulatory element];
eHGT_078 h-minBglobin-hSCN1A-CO-Nterm1049-Intron-CfaN-[post regulatory element] and
eHGT_078 h-minBglobin-CfaC-hSCN1A-CO-Cterm949-Intron-[post regulatory element];
hSyn1-hSCN1A-CO-Nterm956-Intron-CfaN-[post regulatory element] and
hSyn1-CfaC-hSCN1A-CO-Cterm1042-Intron-[post regulatory element]; or
hSyn1-hSCN1A-CO-Nterm947-Intron-CfaN-[post regulatory element] and
hSyn1-CfaC-hSCN1A-CO-Cterm1051-Intron-[post regulatory element].

38. An artificial expression construct comprising a portion of an SCN1A coding sequence, an N-intein coding sequence, and a promoter sequence, wherein the N-intein coding sequence is located at the 3′ end of the portion of the SCN1A coding sequence.

39. The artificial expression construct of claim 38, wherein the portion of the SCN1A coding sequence comprises a sequence having at least 90% sequence identity to SEQ ID NO: 59, SEQ ID NO: 61, or SEQ ID NO: 63.

40. The artificial expression construct of claim 38, wherein the portion of the SCN1A coding sequence comprises hSCN1A-CO-Nterm1049 (SEQ ID NO: 59), hSCN1A-CO-Nterm956 (SEQ ID NO: 61), or hSCN1A-CO-Nterm947 (SEQ ID NO: 63).

41. The artificial expression construct of claim 38, wherein the N-intein coding sequence comprises a Cfa-N coding sequence.

42. The artificial expression construct of claim 41, wherein the Cfa-N coding sequence comprises the sequence as set forth in SEQ ID NO: 57 or a sequence having at least 90% sequence identity to SEQ ID NO: 57.

43. The artificial expression construct of claim 38, wherein the promoter sequence comprises a minBglobin promoter, an hSyn1 promoter, a CMV promoter, or a shortened hSyn1 promoter.

44. The artificial expression construct of claim 38, further comprising an enhancer sequence that leads to targeted expression of the portion of the SCN1A coding sequence within a targeted central nervous system cell type.

45. The artificial expression construct of claim 44, wherein the enhancer sequence comprises DLX2.0 (SEQ ID NO: 2) and the targeted central nervous system cell type comprises a forebrain GABAergic neuron.

46. The artificial expression construct of claim 44, wherein the enhancer sequence comprises eHGT_078 h and the targeted central nervous system cell type comprises a forebrain glutamatergic neuron.

47. The artificial expression construct of claim 38, further comprising an miRNA binding site that leads to targeted expression of the portion of the SCN1A coding sequence within a targeted central nervous system cell type.

48. The artificial expression construct of claim 47, wherein the miRNA binding site comprises a 4×2c miRNA binding site and the targeted central nervous cell type comprises a pan-GABAergic neuron.

49. The artificial expression construct of claim 38, wherein the artificial expression construct further encodes a reporter protein.

50. The artificial expression construct of claim 49, wherein the reporter protein comprises a fluorescent protein or a tag cassette.

51. The artificial expression construct of claim 50, wherein the tag cassette comprises a sequence as set forth in SEQ ID NOs: 15 and 34-45.

52. The artificial expression construct of claim 38, wherein the artificial expression construct is within an adeno-associated viral (AAV) vector.

53. The artificial expression construct of claim 38, wherein the artificial expression construct is associated with a capsid that crosses the blood brain barrier.

54. The artificial expression construct of claim 53, wherein the capsid comprises PHP.eB, AAV-BR1, AAV-PHP.S, AAV-PHP.B, AAV-PPS, AAV1R6, AAV1R7, AAV9, or AAVrh.10.

55. The artificial expression construct of claim 38, wherein the artificial expression construct comprises or encodes a skipping element.

56. The artificial expression construct of claim 55, wherein the skipping element comprises a 2A peptide and/or an internal ribosome entry site (IRES).

57. The artificial expression construct of claim 56, wherein the IRES comprises IRES2.

58. The artificial expression construct of claim 56, wherein the 2A peptide comprises T2A, P2A, E2A, or F2A.

59. The artificial expression construct of claim 38, wherein the artificial expression construct comprises or encodes a set of features selected from: DLX2.0 (SEQ ID NO: 2), 4×2C miR binding site (SEQ ID NO: 56), 8×2C miR binding site (SEQ ID NO: 87), eHGT_078h, intein, split intein (e.g., Cfa-N, Cfa-C), hSCN1A-CO-Nterm1049, hSCN1A-CO-Cterm949, hSCN1A-CO-Nterm956, hSCN1A-CO-Cterm1042, hSCN1A-CO-Nterm947, hSCN1A-CO-Cterm1051, AAV, scAAV, rAAV, pAAV, pSMART-HCKan, minBglobin, CMV promoter, hSyn1 promoter, shortened hSyn1 promoter, minCMV, minCMV*, minRho, minRho* fluorescent protein (e.g., EGFP, SYFP, GFP), hsA2, Cre, iCre, dgCre, FlpO, tTA2, SP10 (e.g., 3×SP10), tag cassette (e.g., 3×FLAG), intron, 10aa, nuclear localization protein, self-cleaving peptides, WPRE, WPRE3, hGHpA, SV40 pA, shortPolyA, and/or BGHpA.

60. An artificial expression construct comprising a portion of an SCN1A coding sequence, a C-intein coding sequence, an enhancer sequence that leads to targeted expression of the portion of the SCN1A coding sequence within a targeted central nervous system cell type, and a promoter sequence, wherein the C-intein coding sequence is located at the 5′ end of the portion of the SCN1A coding sequence.

61. The artificial expression construct of claim 60, wherein the second portion of the SCN1A coding sequence comprises a sequence having at least 90% sequence identity to SEQ ID NO: 60, SEQ ID NO: 62, or SEQ ID NO: 64.

62. The artificial expression construct of claim 60, wherein the second portion of the SCN1A coding sequence comprises hSCN1A-CO-Cterm949 (SEQ ID NO: 60), hSCN1A-CO-Cterm1042, (SEQ ID NO: 62) or hSCN1A-CO-Cterm1051 (SEQ ID NO: 64).

63. The artificial expression construct of claim 60, wherein the C-intein coding sequence comprises a Cfa-C coding sequence.

64. The artificial expression construct of claim 63, wherein the Cfa-C coding sequence comprises the sequence as set forth in SEQ ID NO: 58 or a sequence having at least 90% sequence identity to SEQ ID NO: 58.

65. The artificial expression construct of claim 60, further comprising an enhancer sequence that leads to targeted expression of the portion of the SCN1A coding sequence within a targeted central nervous system cell type.

66. The artificial expression construct of claim 65, wherein the enhancer sequence comprises DLX2.0 (SEQ ID NO: 2) and the targeted central nervous system cell type comprises a forebrain GABAergic neuron.

67. The artificial expression construct of claim 65, wherein the enhancer sequence comprises eHGT_078 h and the targeted central nervous system cell type comprises a forebrain glutamatergic neuron.

68. The artificial expression construct of claim 60, further comprising a miRNA binding site that leads to targeted expression of the portion of the SCN1A coding sequence within a targeted central nervous system cell type.

69. The artificial expression construct of claim 68, wherein the miRNA binding site comprises a 4×2c miRNA binding site and the targeted central nervous cell type comprises a pan-GABAergic neuron.

70. The artificial expression construct of claim 60, wherein the artificial expression construct further encodes a reporter protein.

71. The artificial expression construct of claim 70, wherein the reporter protein comprises a fluorescent protein or a tag cassette.

72. The artificial expression construct of claim 71, wherein the tag cassette comprises a sequence as set forth in SEQ ID NOs: 15 and 34-45.

73. The artificial expression construct of claim 60, wherein the artificial expression construct is within an adeno-associated viral (AAV) vector.

74. The artificial expression construct of claim 60, wherein the artificial expression construct is associated with a capsid that crosses the blood brain barrier.

75. The artificial expression construct of claim 74, wherein the capsid comprises PHP.eB, AAV-BR1, AAV-PHP.S, AAV-PHP.B, AAV-PPS, AAV1R6, AAV1R7, AAV9, or AAVrh.10.

76. The artificial expression construct of claim 60, wherein the artificial expression construct comprises or encodes a skipping element.

77. The artificial expression construct of claim 76, wherein the skipping element comprises a 2A peptide and/or an internal ribosome entry site (IRES).

78. The artificial expression construct of claim 77, wherein the IRES comprises IRES2.

79. The artificial expression construct of claim 77, wherein the 2A peptide comprises T2A, P2A, E2A, or F2A.

80. The artificial expression construct of claim 60, wherein the artificial expression construct comprises or encodes a set of features selected from: DLX2.0 (SEQ ID NO: 2), 4×2C miR binding site (SEQ ID NO: 56), 8×2C miR binding site (SEQ ID NO: 87), eHGT_078h, intein, split intein (e.g., Cfa-N, Cfa-C), hSCN1A-CO-Nterm1049, hSCN1A-CO-Cterm949, hSCN1A-CO-Nterm956, hSCN1A-CO-Cterm1042, hSCN1A-CO-Nterm947, hSCN1A-CO-Cterm1051, AAV, scAAV, rAAV, pAAV, pSMART-HCKan, minBglobin, CMV promoter, hSyn1 promoter, shortened hSyn1 promoter, minCMV, minCMV*, minRho, minRho*, fluorescent protein (e.g., EGFP, SYFP, GFP), hsA2, Cre, iCre, dgCre, FlpO, tTA2, SP10 (e.g., 3×SP10), tag cassette (e.g., 3×FLAG), intron, 10aa, nuclear localization protein, self-cleaving peptides, WPRE, WPRE3, hGHpA, SV40 pA, shortPolyA, and/or BGHpA.

81. An administrable composition comprising the system of claim 2, the artificial expression construct of claim 38, and/or the artificial expression construct of claim 60.

82. A vector comprising the artificial expression construct of claim 38 or the artificial expression construct of claim 60.

83. The vector of claim 82, wherein the vector comprises a viral vector.

84. A vector system comprising the system of claim 2, wherein the first artificial expression construct is within a first vector and the second artificial expression construct is within a second vector.

85. The vector system of claim 84, wherein the first vector comprises the elements of CN3252, CN3683, CN3251, CN3677, CN4541, CN4217, or CN4642.

86. The vector system of claim 84, wherein the second vector comprises the elements of CN3254, CN3684, CN3253, CN3678, CN4542, CN4218, or CN4643.

87. A transgenic cell comprising the system of claim 1.

88. The transgenic cell of claim 87, wherein the transgenic cell comprises a neuron.

89. The transgenic cell of claim 87, wherein the transgenic cell is a GABAergic neuron or a glutamatergic neuron.

90. The transgenic cell of claim 89, wherein the GABAergic neuron is a pan-GABAergic neurons, forebrain GABAergic neurons, hippocampal GABAergic neurons, and/or cortical GABAergic neurons.

91. The transgenic cell of claim 89, wherein the GABAergic neuron is a forebrain GABAergic neuron.

92. The transgenic cell of claim 89, wherein the glutamatergic neuron comprises a forebrain glutamatergic neuron.

93. The transgenic cell of claim 87, wherein the transgenic cell comprises a cell line.

94. The transgenic cell of claim 87, wherein the transgenic cell is murine, human, or non-human primate.

95. A non-human transgenic animal comprising the system of claim 2 and/or the transgenic cell of claim 87.

96. The non-human transgenic animal of claim 95, wherein the non-human transgenic animal is a mouse or a non-human primate.

97. A kit comprising the system of claim 2, the artificial expression construct of claim 38, the artificial expression construct of claim 60, the transgenic cell of claim 87, and/or the non-human transgenic animal of claim 95.

98. A method of rescuing voltage-gated sodium channel function within a targeted population of cells, the method comprising co-administering a therapeutically effective amount of the artificial expression construct of claim 38 and a therapeutically effective amount of the artificial expression construct of claim 60, to a sample or subject comprising the targeted population of cells thereby rescuing voltage-gated sodium channel function within the targeted population of cells.

99. The method of claim 98, wherein the co-administering comprises pipetting.

100. The method of claim 99, wherein the pipetting is to a brain slice.

101. The method of claim 100, wherein the brain slice comprises a neuron.

102. The method of claim 100, wherein the brain slice comprises a GABAergic neuron or a glutamatergic neurons.

103. The method of claim 102, wherein the GABAergic neuron comprises a pan-GABAergic neuron, a forebrain GABAergic neuron, a hippocampal GABAergic neuron, or a cortical GABAergic neuron.

104. The method of claim 102, wherein the glutamatergic neuron comprises a forebrain glutamatergic neuron.

105. The method of claim 100, wherein the brain slice comprises a cell line.

106. The method of claim 100, wherein the brain slice is murine, human, or non-human primate.

107. The method of claim 98, wherein the co-administering comprises administering to a living subject.

108. The method of claim 107, wherein the living subject is a human, non-human primate, or a mouse.

109. The method of claim 107, wherein the living subject is in need thereof due to a diagnosis of an SCN1A-related seizure disorder.

110. The method of claim 109, wherein the SCN1A-related seizure disorder comprises Dravet syndrome, myoclonic seizures, myoclonic astatic epilepsy (MAE), intractable childhood epilepsy with generalized tonic-clonic seizures, simple febrile seizures, generalized epilepsy and febrile seizures plus (GEFS+), migrating partial seizures of infancy, Lennox-Gastaut syndrome, or West syndrome.

111. The method of claim 107, wherein the living subject is a pediatric patient.

112. The method of claim 107, wherein the living subject is less than 4 years old.

113. The method of claim 107, wherein the living subject is a transgenic non-human animal.

114. The method of claim 98, wherein the co-administering comprises intravenous injection, intraparenchymal injection into brain tissue, intracerebroventricular (ICV) injection, intra-cisterna magna (ICM) injection, or intrathecal injection.

115. An artificial expression construct comprising CN3252, CN3254, CN3683, CN3684, CN3251, CN3253, CN3677, CN3678, CN4541, CN4542, CN4217, CN4218, CN4642, or CN4643.
